(19) United States
(12) Patent Application Publication
(10) Pub. No.: US 2025/0067755 A1
(43) Pub. Date: Feb. 27, 2025

(54) USE OF ANTI-FAMILY WITH SEQUENCE SIMILARITY 19, MEMBER A5 ANTIBODIES FOR THE TREATMENT AND DIAGNOSIS OF MOOD DISORDERS

(71) Applicant: Neuracle Science Co., Ltd., Seoul (KR)

(72) Inventors: Jae Young SEONG, Seoul (KR); Byung Joo HAM, Seoul (KR); Min-Hyeok LEE, Seoul (KR); Nui HA, Gyeonggi-do (KR)

(73) Assignee: Neuracle Science Co., Ltd., Seoul (KR)

(21) Appl. No.: 18/776,405

(22) Filed: Jul. 18, 2024

Related U.S. Application Data
(62) Division of application No. 16/753,295, filed on Apr. 2, 2020, now Pat. No. 12,072,339.
(60) Provisional application No. 62/566,877, filed on Oct. 2, 2017.
(60) Provisional application No. 62/582,888, filed on Nov. 7, 2017.

(51) Int. Cl. G01N 33/68; C07K 16/24; C12Q 1/6883

(52) U.S. Cl. CPC G01N 33/6896; C07K 16/24; C12Q 1/6883; C07K 2317/622; C12Q 2600/158; G01N 2800/304

(57) ABSTRACT

The present disclosure relates to a method of diagnosing a mood disorder in a subject comprising measuring serum FAM19A5 concentration with an antagonist (e.g., an antibody or antigen-binding fragment thereof) that specifically binds to FAM19A5. The present disclosure also relates to a method of treating a mood disorder in a subject in need thereof comprising administering to the subject an FAM19A5 antagonist.

The content of the electronically submitted sequence listing in ASCII text file (Name: 3763.007PC02_ST25.txt; Size: 166,984 bytes; and Date of Creation: Oct. 1, 2018) filed with the application is incorporated herein by reference in its entirety.

### FIELD OF THE DISCLOSURE

The present disclosure provides methods for diagnosing and/or treating mood disorders, such as major depressive disorder and bipolar disorder, in a subject (e.g., a human) using antagonists (e.g., antibodies or an antigen-binding fragment thereof) that specifically bind to family with sequence similarity 19, member A5 (FAM19A5) or a composition comprising such antagonists. The present disclosure also provides methods of identifying a subject having a mood disorder using the FAM19A5 antagonists.

### BACKGROUND OF THE DISCLOSURE

Mood disorders are a category of mental illnesses that primarily affect an individual's emotional state. Based on the underlying symptoms, mood disorders can be categorized into three basic groups: (i) elevated mood (mania or hypomania); (ii) depression; and (iii) moods which cycle between mania and depression. Major types of mood disorders include: major depressive disorder, dysthymia (chronic, low-grade, depressed and/or irritable mood that lasts for at least two years), mood disorders related to another health condition (e.g., cancer, injuries, infections, and chronic illnesses), and substance-induced (e.g., medication, drug abuse, alcoholism, exposure to toxins) mood disorders.

Major depressive disorder (MDD), also known as major depression, clinical depression, and unipolar depression, is the most common of the mood disorders and affects more than 200 million people worldwide. Vos T et al., Lancet 388:1545-1602 (2016). Bipolar disorder (BD), also known as manic-depressive illness or manic depression, is associated with recurrent episodes of extreme mood swings that include emotional highs (mania or hypomania) and lows (depression). Anderson I. M. et al., BMJ 345:e8508 (2012). BD can affect individuals of all ages (although median age of onset is 25 years) and is the sixth leading cause of disability in the world.

While the exact causes of mood disorders remains unknown, it is generally thought that an imbalance in the level of neurotransmitters (e.g., dopamine, norepinephrine, or serotonin) plays an important role. Manji H. K. et al., World Psychiatry 2:136-46 (2003); Nutt D. J., J Cin Psychiatry 69 Suppl E1:4-7 (2008). Neuroimaging and postmortem studies have also suggested an association between mood disorders and significant abnormalities in the cellular composition of certain regions of the brain (e.g., subgenual anterior cingulate cortex, hippocampus, frontal cortex, subgenual prefrontal cortex, striaturm, and white matter). Harrison P. J. et al., Brain 125:1428-1449 (2002); Banasr M et al., Curr Opin Cell Biol. 23:730-737 (2011).

Currently, there is no precise quantitative method of diagnosing mood disorders. Current methods are largely qualitative and based on healthcare professional's subjective assessment of an individual's behavior and/or information provided by the individual or caregivers. Such approaches have resulted in the improper diagnosis of many patients. Ruggero C. J. et al., J Clin Psychiatry 72:1207-13 (2011). Accordingly, there is a current need for more effective treatment and diagnostic options for mood disorders.

### BRIEF SUMMARY OF THE DISCLOSURE

Provided herein is a method of diagnosing a mood disorder in a subject in need thereof comprising contacting an antagonist against a family with sequence similarity 19, member A5 (FAM19A5) (“FAM19A5 antagonist”) with a biological sample of the subject and measuring a FAM19A5 protein level or a FAM19A5 mRNA level in the sample. In some embodiments, the contacting and the measuring is performed in vitro.

In some embodiments, the subject exhibits a higher protein level of FAM19A5 or a higher nucleic acid level encoding FAM19A5 compared to a reference protein level of FAM19A5 (the protein level of FAM19A5 in a sample of a subject who does not have the mood disorder) or a reference nucleic acid level encoding FAM19A5 (nucleic acid level encoding FAM19A5 in a sample of a subject who does not have the mood disorder).

In some embodiments, the protein level of FAM19A5 is measured by an immunohistochemistry, a Western blotting, a radioimmunoassay, an enzyme linked immunosorbent assay (ELISA), a radioimmunodiffusion, an immunoprecipitation assay, an Ouchterlony immunodiffusion method, a rocket immunoelectrophoresis, a tissue immunostaining method, a complement fixation assay, FACS, a protein chip, or any combination thereof. In some embodiments, the nucleic acid level encoding FAM19A5 is measured by a RT-PCT, a real time polymerase chain reaction, or a Northern blot.

In some embodiments, the biological sample comprises a tissue, cell, blood, serum, plasma, saliva, urine, cerebral spinal fluid (CSF), or any combination thereof.

Also disclosed herein is an FAM19A5 antagonist for treating a mood disorder in a subject in need thereof.

Present disclosure also provides an FAM19A5 antagonist for increasing thickness of a cortical gyrus in a subject having a mood disorder. In some embodiments, the cortical gyrus comprises a transverse frontopolar gyrus, dorsal posterior cingulate gyrus, pars opercualris, pars triangularis, middle frontal gyrus, fusiform gyrus, lingual gyrus, planum temporale, cuneus, pars orbitalis, anterior transverse temporal gyrus, planum temporale, or any combinations thereof.

In some embodiments, a mood disorder (e.g., concerning the methods and/or FAM19A5 antagonist for use disclosed herein) comprises a major depressive disorder (MDD), bipolar disorder (BD), minor depressive disorder, persistent depressive disorder (dysthymia), seasonal affective disorder (SAD), psychotic depression, postpartum depression, premenstrual dysphoric disorder (PMDD), situational depression, atypical depression, anxiety disorder, or any combination thereof.

In some embodiments, an FAM19A5 antagonist is an antisense oligonucleotide, an siRNA, an shRNA, a miRNA, a dsRNA targeting FAM19A5, an aptamer, a PNA, a vector including the same, or any combinations thereof. In other embodiments, an FAM19A5 antagonist is an antibody that specifically binds to FAM19A5 protein (“anti-FAM19A5 antibody”).

In some embodiments, an anti-FAM19A5 antibody comprises a heavy chain CDR1, CDR2, and CDR3, and a light chain CDR1, CDR2, and CDR3, wherein
(i) the heavy chain CDR1, CDR2, and CDR3 comprises the amino acid sequences set forth as SEQ ID NOs: 11, 12, and 13, respectively, and the light chain CDR1, CDR2, and CDR3 comprises the amino acid sequences set forth as SEQ ID NOs: 23, 24, and 25, respectively;
(ii) the heavy chain CDR1, CDR2, and CDR3 comprises the amino acid sequences set forth as SEQ ID NOs: 14, 15, and 16, respectively, and the light chain CDR1, CDR2, and CDR3 comprises the amino acid sequences set forth as SEQ ID NOs: 26, 27, and 28, respectively;
(iii) the heavy chain CDR1, CDR2, and CDR3 comprises the amino acid sequences set forth as SEQ ID NOs: 17, 18, and 19, respectively, and the light chain CDR1, CDR2, and CDR3 comprises the amino acid sequences set forth as SEQ ID NOs: 29, 30, and 31, respectively; or
(iv) the heavy chain CDR1, CDR2, and CDR3 comprises the amino acid sequences set forth as SEQ ID NOs: 20, 21, and 22, respectively, and the light chain CDR1, CDR2, and CDR3 comprises the amino acid sequences set forth as SEQ ID NOs: 32, 33, and 34, respectively.

In some embodiments, an anti-FAM19A5 antibody comprises a heavy chain variable region (VH) and a light chain variable region (VL), wherein
(i) the VH comprises the amino acid sequence set forth as SEQ ID NO: 35 and the VL comprises the amino acid sequence set forth as SEQ ID NO: 39;
(ii) the VH comprises the amino acid sequence set forth as SEQ ID NO: 36 and the VL comprises the amino acid sequence set forth as SEQ ID NO: 40;
(iii) the VH comprises the amino acid sequence set forth as SEQ ID NO: 37 and the VL comprises the amino acid sequence set forth as SEQ ID NO: 41; or
(iv) the VH comprises the amino acid sequence set forth as SEQ ID NO: 38 and the VL comprises the amino acid sequence set forth as SEQ ID NO: 42

In some embodiments, an anti-FAM19A5 antibody comprises a heavy chain variable region (VH) and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NO: 35, 36, 37, or 38; and wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NO: 39, 40, 41, or 42.

In some embodiments, an anti-FAM19A5 antibody is a chimeric antibody, a humanized antibody, or a human antibody.

### EMBODIMENTS

Embodiment 1. A method of diagnosing a mood disorder in a subject in need thereof comprising measuring a protein level of a family with sequence similarity 19, member A5 (FAM19A5) or a nucleic acid level encoding FAM19A5 in a sample of the subject.
Embodiment 2. A method of identifying a subject having a mood disorder comprising measuring a protein level of FAM19A5 or a nucleic acid level encoding FAM19A5 in a sample of the subject.
Embodiment 3. The method of Embodiment 1 or 2, wherein the measuring comprises contacting an anti-FAM19A5 antibody, or antigen-binding fragment thereof, with the sample of the subject.
Embodiment 4. The method of Embodiment 1 or 2, wherein the nucleic acid level encoding FAM19A5 is measured by a nucleotide sequence that is complementary to the nucleic acid.
Embodiment 5. The method of any one of Embodiments 1 to 3, wherein the protein level of FAM19A5 is measured by an immunohistochemistry, a Western blotting, a radioimmunoassay, an enzyme linked immunosorbent assay (ELISA), a radioimmunodiffusion, an immunoprecipitation assay, an Ouchterlony immunodiffusion method, a rocket immunoelectrophoresis, a tissue immunostaining method, a complement fixation assay, FACS, a protein chip, or any combination thereof.
Embodiment 6. The method of any one of Embodiments 1, 2, and 4, wherein the nucleic acid level encoding FAM19A5 is measured by a RT-PCT, a real time polymerase chain reaction, or a Northern blot.
Embodiment 7. The method of any one of Embodiments 1 to 6, wherein the sample comprises a tissue, cell, blood, serum, plasma, saliva, urine, cerebral spinal fluid (CSF), or any combination thereof.
Embodiment 8. The method of any one of Embodiments 1 to 7, wherein the subject exhibits a higher protein level of FAM19A5 or a higher nucleic acid level encoding FAM19A5 compared to the reference protein level of FAM19A5 (the protein level of FAM19A5 in a sample of a subject who does not have the mood disorder) or the reference nucleic acid level encoding FAM19A5 (nucleic acid level encoding FAM19A5 in a sample of a subject who does not have the mood disorder).
Embodiment 9. The method of Embodiment 8, wherein the subject exhibits at least 1.1 fold, at least 1.2 fold, at least 1.3 fold, at least 1.4 fold, at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 15 fold, at least 20 fold, at least 25 fold, or at least 30 fold increase in the protein level of FAM19A5 or the nucleic acid level encoding FAM19A5.
Embodiment 10. The method of any one of Embodiments 1, 2, 3, 5, and 7 to 9, wherein the protein level of FAM19A5 is measured by an ELISA.
Embodiment 11. The method of Embodiment 10, wherein the reference protein level of FAM19A5 is about 2.0 ng/ml, about 1.9 ng/ml, about 1.8 ng/ml, about 1.7 ng/ml, about 1.6 ng/ml, about 1.5 ng/ml, about 1.4 ng/ml, about 1.3 ng/ml, about 1.2 ng/ml, about 1.1 ng/ml, about 1 ng/ml, about 0.9 ng/ml, about 0.8 ng/ml, about 0.7 ng/ml, about 0.6 ng/ml, about 0.5 ng/ml, about 0.4 ng/ml, about 0.3 ng/ml, about 0.2 ng/ml, about 0.1 ng/ml, or lower as measured by an ELISA.
Embodiment 12. The method of Embodiment 11, wherein the reference protein level of FAM19A5 is about 2.0 ng/ml or lower as measured by an ELISA.
Embodiment 13. The method of any one of Embodiments 9 to 12, wherein the increased protein level of FAM19A5 or the increased nucleic acid level encoding FAM19A5 is associated with a decrease in thickness of a cortical gyrus of the subject.
Embodiment 14. The method of any one of Embodiments 1 to 13, wherein the subject is treated with an anti-depressant agent or an antipsychotic agent after the diagnosing or the identifying.
Embodiment 15. The method of Embodiment 14, wherein the anti-depressant agent comprises selective serotonin reuptake inhibitors (SSRIs), serotonin-norepinephrine reuptake inhibitors (SNRIs), serotonin modulators and stimulators (SMSs), serotonin antagonists and reuptake inhibitors (SARIs), norepinephrine reuptake inhibitors (NRIs), tricyclic antidepressants (TCAs), tetracyclic antidepressants (TeCAs), monoamine oxidase inhibitors (MAOIs), or any combination thereof
Embodiment 16. The method of Embodiment 15, wherein the SSRIs comprises citalopram (CELEXA®), escitalopram (LEXAPRO®, CIPRALEX®), paroxetine (PAXIL®, SEROXAT®), fluoxetine (PROZAC®), fluvoxamine (LUVOX®), sertraline (ZOLOFT®, LUSTRAL®), zimelidine (NORMUD®, ZELMID®), indalpine (UPSTENE®), or any combination thereof.
Embodiment 17. The method of Embodiment 15, wherein the SNRIs comprises desvenlafaxine (PRISTIQ®), duloxetine (CYMBALTA®), levomilnacipran (FETZIMA®), milnacipran (IXEL®, SAVELLA®), tofenacin (ELAMOL®, TOFACINE®), venlafaxine (EFFEXOR®), or any combination thereof.
Embodiment 18. The method of Embodiment 15, wherein the SMSs comprises vilazodone (VIIBRYD®), vortioxetine (TRINTELLIX®) or any combination thereof.
Embodiment 19. The method of Embodiment 15, wherein the SARIs comprises etoperidone (AXIOMIN®, ETONIN®), nefazodone (NEFADAR®, SERZONE®), trazodone (DESYREL®), or any combination thereof.
Embodiment 20. The method of Embodiment 15, wherein the NRIs comprises reboxetine (EDRONAX®), viloxazine (VIVALAN®), atomoxetine (STRATTERA®), or any combination thereof.
Embodiment 21. The method of Embodiment 15, wherein the TCAs comprises amitriptyline (ELAVIL®, ENDEP®), amitriptylinoxide (AMIOXID®, AMBIVALON®, EQUILIBRIN®), clomipramine (ANAFRANIL®), desipramine (NORPRAMIN®, PERTOFRANE®), dibenzepin (NOVERIL®, VICTORIL®), dimetacrine (ISTONIL®), dosulepin (PROTHIADEN®), doxepin (ADAPIN®, SINEQUAN®), imipramine (TOFRANIL®), lofepramine (LOMONT®, GAMANIL®), melitracen (DIXERAN®, MELIXERAN®, TRAUSABUN®), nitroxazepine (SINTAMIL®), nortriptyline (PAMELOR®, AVENTYL®), noxiptiline (AGEDAL®, ELRONON®, NOGEDAL®), pipofezine (AZAFEN®/AZAPHEN®), protriptyline (VIVACTIL®), trimipramine (SURMONTIL®), butriptyline (EVADYNE®), demexiptiline (DEPARON®, TINORAN®), imipraminoxide (IMIPREX®, ELEPSIN®), iprindole (PRONDOL®, GALATUR®, TETRAN®), metapramine (TIMAXEL®), propizepine (DEPRESSIN®, VAGRAN®), quinupramine (KINUPRIL®, KEVOPRIL®), opipramol (INSIDON®), tianeptine (STABLON®), amineptine (SURVECTOR®, MANEON®), tiazesim (ALTINIL®), or any combination thereof.
Embodiment 22. The method of Embodiment 15, wherein the TeCAs comprises amoxapine (ASENDIN®), maprotiline (LUDIOMIL®), mianserin (BOLVIDON®, NORVAL®, TOLVON®), mirtazapine (REMERON®), setiptiline (TECIPUL®), or any combination thereof.
Embodiment 23. The method of Embodiment 15, wherein the MAOIs comprises isocarboxazid (MARPLAN®), phenelzine (NARDIL®), tranylcypromine (PARNATE®), benmoxin (NEURALEX®), iproclozide (SURSUM®), iproniazid (MARSTLID®), mebanazine (ACTOMOL®), nialamide (NIAMID®), octamoxin (XIMAOL®), pheniprazine (CATRON®), phenoxypropazine (DRAZINE®), pivhydrazine (TERSAVID®), safrazine (SAFRA®), selegiline (ELDEPRYL®, ZELAPAR®, EMSAM®), caroxazone (SURODIL®, TIMOSTENIL®), metralindole (INKAZAN®), moclobemide (AURORIX®, MANERIX®), pirlindole (PIRAZIDOL®), toloxatone (HUMORYL®), eprobemide (BEFOL®), minaprine (BRANTUR®, CANTOR®), or any combination thereof.
Embodiment 24. The method of Embodiment 14, wherein the antipsychotic agent comprises a typical antipsychotic or an atypical antipsychotic agent.
Embodiment 25. The method of Embodiment 24, wherein the typical antipsychotic agent comprises chlorpromazine (THORAZINE®, LARGACTIL®), chlorprothixene (TARACTAN®), levomepromazine (NOZINAN®, LEVOPROME®), mesoridazine (SERENTIL®), periciazine (NEULEPTIL®), promazine (SPARINE®), thioridazine (MELLARIL®), loxapine (LOXITANE®), molindone (MOBAN®), perphenazine (TRILAFON®), thiothixene (NAVANE®), droperidol (INAPSINE®), flupentixol (DEPIXOL®, FLUANXOL®), fluphenazine (PROLIXIN®), haloperidol (HALDOL®), pimozide (ORAP®), prochlorperazine (COMPAZINE®), thioproperazine (MAJEPTIL®), trifluoperazine (STELAZINE®), zuclopenthixol (CLOPIXOL®), or any combination thereof.
Embodiment 26. The method of Embodiment 24, wherein the atypical antipsychotic agent comprises amisulpride (SOLIAN®), lurasidone (LATUDA®), quetiapine (SEROQUEL®), risperidone (RISPERDAL®), olanzapine (ZYPREXA®), aripiprazole (ABILIFY®), ziprasidone (GEODON®), pimavanserin (NUPLAZID®), clozapine (CLOZARIL®), paliperidone (INVEGA®), asenapine (SAPHRIS®), brexpiprazole (REXULTI®), cariprazine (VRAYLAR®), iloperidone (FANAPT®), or any combination thereof.
Embodiment 27. The method of Embodiment 14, wherein the anti-depressant agent is a FAM19A5 antagonist.
Embodiment 28. A method for treating a mood disorder in a subject in need thereof comprising administering to the subject a FAM19A5 antagonist.
Embodiment 29. A method of reducing, inhibiting, or ameliorating one or more symptoms of a mood disorder in a subject in need thereof comprising administering to the subject a FAM19A5 antagonist.
Embodiment 30. The method of any one of Embodiments 1 to 29, wherein the mood disorder is selected from the group consisting of a major depressive disorder (MDD), bipolar disorder (BD), minor depressive disorder, persistent depressive disorder (dysthymia), seasonal affective disorder (SAD), psychotic depression, postpartum depression, premenstrual dysphoric disorder (PMDD), situational depression, atypical depression, anxiety disorder, or any combination thereof.
Embodiment 31. The method of any one of Embodiments 28 to 30, wherein the mood disorder is associated with or caused by a decrease in thickness of a cortical gyrus of the subject.
Embodiment 32. A method for increasing thickness of a cortical gyrus in a subject having a mood disorder comprising administering to the subject a FAM19A5 antagonist.
Embodiment 33. The method of any one of Embodiments 13 to 32, wherein the cortical gyrus is selected from the group consisting of a transverse frontopolar gyrus, dorsal posterior cingulate gyrus, pars opercualris, pars triangularis, middle frontal gyrus, fusiform gyrus, lingual gyrus, planum temporale, cuneus, pars orbitalis, anterior transverse temporal gyrus, planum temporale, and any combination thereof.
Embodiment 34. The method of any one of Embodiments 1 to 33, wherein the mood disorder is major depressive disorder (MDD).
Embodiment 35. The method of any one of Embodiments 1 to 33, wherein the mood disorder is bipolar disorder (BD).
Embodiment 36. The method of any one of Embodiments 28 to 35, wherein the mood disorder is associated with an increase in a protein level of FAM19A5 or a nucleic acid level encoding FAM19A5 protein in the subject.
Embodiment 37. The method of any one of Embodiments 27 to 36, wherein the FAM19A5 antagonist is an antisense oligonucleotide, an siRNA, an shRNA, a miRNA, a dsRNA targeting FAM19A5, an aptamer, a PNA, a vector including the same, or any combination thereof.
Embodiment 38. The method of any one of Embodiments 27 to 36, wherein the FAM19A5 antagonist is an anti-FAM19A5 antibody, a polynucleotide encoding the anti-FAM19A5 antibody, a vector comprising the polynucleotide thereof, a cell comprising the polynucleotide thereof, or any combination thereof.
Embodiment 39. The method of Embodiment 38, wherein the FAM19A5 antagonist is an anti-FAM19A5 antibody.
Embodiment 40. The method of Embodiment 39, wherein the anti-FAM19A5 antibody exhibits a property selected from:
(a) binds to soluble human FAM19A5 with a K_{D} of 10 nM or less as measured by ELISA;
(b) binds to membrane bound human FAM19A5 with a K_{D} of 10 nM or less as measured by ELISA; or
(c) both (a) and (b).
Embodiment 41. The method of Embodiment 39 or 40, wherein the anti-FAM19A5 antibody cross-competes for binding to a human FAM19A5 epitope with a reference antibody comprising a heavy chain CDR1, CDR2, and CDR3 and a light chain CDR1, CDR2, and CDR3,
(i) wherein the heavy chain CDR1 comprises the amino acid sequence of SEQ ID NO: 11, the heavy chain CDR2 comprises the amino acid sequence of SEQ ID NO: 12, the heavy chain CDR3 comprises the amino acid sequence of SEQ ID NO: 13, the light chain CDR1 comprises the amino acid sequence of SEQ ID NO: 23, the light chain CDR2 comprises the amino acid sequence of SEQ ID NO: 24, and the light chain CDR3 comprises the amino acid sequence of SEQ ID NO: 25;
(ii) wherein the heavy chain CDR1 comprises the amino acid sequence of SEQ ID NO: 14, the heavy chain CDR2 comprises the amino acid sequence of SEQ ID NO: 15, the heavy chain CDR3 comprises the amino acid sequence of SEQ ID NO: 16, the light chain CDR1 comprises the amino acid sequence of SEQ ID NO: 26, the light chain CDR2 comprises the amino acid sequence of SEQ ID NO: 27, and the light chain CDR3 comprises the amino acid sequence of SEQ ID NO: 28;
(iii) wherein the heavy chain CDR1 comprises the amino acid sequence of SEQ ID NO: 17, the heavy chain CDR2 comprises the amino acid sequence of SEQ ID NO: 18, the heavy chain CDR3 comprises the amino acid sequence of SEQ ID NO: 19, the light chain CDR1 comprises the amino acid sequence of SEQ ID NO: 29, the light chain CDR2 comprises the amino acid sequence of SEQ ID NO: 30, and the light chain CDR3 comprises the amino acid sequence of SEQ ID NO: 31; or
(iv) wherein the heavy chain CDR1 comprises the amino acid sequence of SEQ ID NO: 20, the heavy chain CDR2 comprises the amino acid sequence of SEQ ID NO: 21, the heavy chain CDR3 comprises the amino acid sequence of SEQ ID NO: 22, the light chain CDR1 comprises the amino acid sequence of SEQ ID NO: 32, the light chain CDR2 comprises the amino acid sequence of SEQ ID NO: 33, and the light chain CDR3 comprises the amino acid sequence of SEQ ID NO: 34.
Embodiment 42. The method of any one of Embodiments 39 to 41, wherein the anti-FAM19A5 antibody binds to the same FAM19A5 epitope as a reference antibody comprising a heavy chain CDR1, CDR2, and CDR3 and a light chain CDR1, CDR2, and CDR3, (i) wherein the heavy chain CDR1 comprises the amino acid sequence of SEQ ID NO: 11, the heavy chain CDR2 comprises the amino acid sequence of SEQ ID NO: 12, the heavy chain CDR3 comprises the amino acid sequence of SEQ ID NO: 13, the light chain CDR1 comprises the amino acid sequence of SEQ ID NO: 23, the light chain CDR2 comprises the amino acid sequence of SEQ ID NO: 24, and the light chain CDR3 comprises the amino acid sequence of SEQ ID NO: 25;
(ii) wherein the heavy chain CDR1 comprises the amino acid sequence of SEQ ID NO: 14, the heavy chain CDR2 comprises the amino acid sequence of SEQ ID NO: 15, the heavy chain CDR3 comprises the amino acid sequence of SEQ ID NO: 16, the light chain CDR1 comprises the amino acid sequence of SEQ ID NO: 26, the light chain CDR2 comprises the amino acid sequence of SEQ ID NO: 27, and the light chain CDR3 comprises the amino acid sequence of SEQ ID NO: 28;
(iii) wherein the heavy chain CDR1 comprises the amino acid sequence of SEQ ID NO: 17, the heavy chain CDR2 comprises the amino acid sequence of SEQ ID NO: 18, the heavy chain CDR3 comprises the amino acid sequence of SEQ ID NO: 19, the light chain CDR1 comprises the amino acid sequence of SEQ ID NO: 29, the light chain CDR2 comprises the amino acid sequence of SEQ ID NO: 30, and the light chain CDR3 comprises the amino acid sequence of SEQ ID NO: 31; or
(iv) wherein the heavy chain CDR1 comprises the amino acid sequence of SEQ ID NO: 20, the heavy chain CDR2 comprises the amino acid sequence of SEQ ID NO: 21, the heavy chain CDR3 comprises the amino acid sequence of SEQ ID NO: 22, the light chain CDR1 comprises the amino acid sequence of SEQ ID NO: 32, the light chain CDR2 comprises the amino acid sequence of SEQ ID NO: 33, and the light chain CDR3 comprises the amino acid sequence of SEQ ID NO: 34.
Embodiment 43. The method of any one of Embodiments 39 to 42, wherein the anti-FAM19A5 antibody binds to at least one FAM19A5 epitope, which is SEQ ID NO: 6 or SEQ ID NO: 9.
Embodiment 44. The method of any one of Embodiments 39 to 42, wherein the anti-FAM19A5 antibody binds only to an FAM19A5 epitope, which is SEQ ID NO: 6 or SEQ ID NO: 9.
Embodiment 45. The method of any one of Embodiments 39 to 43, wherein the anti-FAM19A5 antibody further binds to an additional FAM19A5 epitope.
Embodiment 46. The method of Embodiment 45, wherein the additional FAM19A5 epitope is selected from the group consisting of SEQ ID NO: 5, SEQ ID NO: 6, SEQ ID NO: 7, SEQ ID NO: 8, SEQ ID NO: 9, SEQ ID NO: 10, and any combination thereof.
Embodiment 47. The method of any one of Embodiments 39 to 46, wherein the anti-FAM19A5 antibody comprises a heavy chain CDR1, CDR2, and CDR3 and a light chain CDR1, CDR2, and CDR3.
Embodiment 48. The method of Embodiment 47, wherein the heavy chain CDR3 of the anti-FAM19A5 antibody comprises the amino acid sequence of SEQ ID NO: 13, SEQ ID NO: 16, SEQ ID NO: 19, or SEQ ID NO: 22.
Embodiment 49. The method of Embodiment 47 or 48, wherein the heavy chain CDR1 of the anti-FAM19A5 antibody comprises the amino acid sequence of SEQ ID NO: 11, SEQ ID NO: 14, SEQ ID NO: 17, or SEQ ID NO: 20.
Embodiment 50. The method of any one of Embodiments 47 to 49, wherein the heavy chain CDR2 of the anti-FAM19A5 antibody comprises the amino acid sequence of SEQ ID NO: 12, SEQ ID NO: 15, SEQ ID NO: 18, or SEQ ID NO: 21.
Embodiment 51. The method of any one of Embodiments 47 to 50, wherein the light chain CDR1 of the anti-FAM19A5 antibody comprises the amino acid sequence of SEQ ID NO: 23, SEQ ID NO: 26, SEQ ID NO: 29, or SEQ ID NO: 32.
Embodiment 52. The method of any one of Embodiments 47 to 51, wherein the light chain CDR2 of the anti-FAM19A5 antibody comprises the amino acid sequence of SEQ ID NO: 24, SEQ ID NO: 27, SEQ ID NO: 30, or SEQ ID NO: 33.
Embodiment 53. The method of any one of Embodiments 47 to 52, wherein the light chain CDR3 of the anti-FAM19A5 antibody comprises the amino acid sequence of SEQ ID NO: 25, SEQ ID NO: 28, SEQ ID NO: 31, or SEQ ID NO: 34.
Embodiment 54. The method of Embodiment 47, wherein the anti-FAM19A5 antibody comprises a heavy chain CDR1, CDR2, and CDR3, and a light chain CDR1, CDR2, and CDR3, wherein (i) the heavy chain CDR1, CDR2, and CDR3 comprises SEQ ID NOs: 11, 12, and 13, respectively, and the light chain CDR1, CDR2, and CDR3 comprises SEQ ID NOs: 23, 24, and 25, respectively;
(ii) the heavy chain CDR1, CDR2, and CDR3 comprises SEQ ID NOs: 14, 15, and 16, respectively, and the light chain CDR1, CDR2, and CDR3 comprises SEQ ID NOs: 26, 27, and 28, respectively;
(iii) the heavy chain CDR1, CDR2, and CDR3 comprises SEQ ID NOs: 17, 18, and 19, respectively, and the light chain CDR1, CDR2, and CDR3 comprises SEQ ID NOs: 29, 30, and 31, respectively; or
(iv) the heavy chain CDR1, CDR2, and CDR3 comprises SEQ ID NOs: 20, 21, and 22, respectively, and the light chain CDR1, CDR2, and CDR3 comprises SEQ ID NOs: 32, 33, and 34, respectively.
Embodiment 55. The method of any one of Embodiments 39 to 47, wherein the anti-FAM19A5 antibody comprises a heavy chain variable domain comprising SEQ ID NO: 35, SEQ ID NO: 36, SEQ ID NO: 37, or SEQ ID NO: 38 and/or a light chain variable domain comprising SEQ ID NO: 39, SEQ ID NO: 40, SEQ ID NO: 41, or SEQ ID NO: 42.
Embodiment 56. The method of Embodiment 55, wherein the anti-FAM19A5 antibody comprises a heavy chain variable domain comprising SEQ ID NO: 35 and a light chain variable domain comprising SEQ ID NO: 39.
Embodiment 57. The method of Embodiment 55, wherein the anti-FAM19A5 antibody comprises a heavy chain variable domain comprising SEQ ID NO: 36 and a light chain variable domain comprising SEQ ID NO: 40.
Embodiment 58. The method of Embodiment 55, wherein the anti-FAM19A5 antibody comprises a heavy chain variable domain comprising SEQ ID NO: 37 and a light chain variable domain comprising SEQ ID NO: 41.
Embodiment 59. The method of Embodiment 55, wherein the anti-FAM19A5 antibody comprises a heavy chain variable domain comprising SEQ ID NO: 38 and a light chain variable domain comprising SEQ ID NO: 42.
Embodiment 60. The method of any one of Embodiments 39 to 47, wherein the anti-FAM19A5 antibody comprises a heavy chain variable region and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NO: 35, 36, 37, or 38.
Embodiment 61. The method of any one of Embodiments 39 to 47, wherein the anti-FAM19A5 antibody comprises a heavy chain variable region and a light chain variable region, wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NO: 39, 40, 41, or 42.
Embodiment 62. The method of any one of Embodiments 39 to 47, wherein the anti-FAM19A5 antibody comprises a heavy chain variable region and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NO: 35, 36, 37, or 38; and wherein the light chain variable region comprises an amino acid sequence which is at least about 8000, at least about 85%, at least about 90%, at least about 9500, at least about 96%, at least about 97%, at least about 98%, at least about 99%, or about 100% identical to the amino acid sequence set forth as SEQ ID NO: 39, 40, 41, or 42.
Embodiment 63. The method of any one of Embodiments 39 to 62, wherein the anti-FAM19A5 antibody is a chimeric antibody, a human antibody, or a humanized antibody.
Embodiment 64. The method of any one of Embodiments 39 to 63, wherein the anti-FAM19A5 antibody comprises an Fab, an Fab′, an F(ab′)2, an Fv, or a single chain Fv (scFv).
Embodiment 65. The method of any one of Embodiments 39 to 64, wherein the anti-FAM19A5 antibody is selected from the group consisting of an IgG1, an IgG2, an IgG3, an IgG4, a variant thereof, and any combination thereof.
Embodiment 66. The method of Embodiment 65, wherein the anti-FAM19A5 antibody is an IgG2, an IgG4, or a combination thereof.
Embodiment 67. The method of Embodiment 65, wherein the anti-FAM19A5 antibody comprises an IgG2/IgG4 isotype antibody.
Embodiment 68. The method of any one of Embodiments 39 to 64, wherein the anti-FAM19A5 antibody further comprises a constant region without the Fc function.
Embodiment 69. The method of any of Embodiments 39 to 68, wherein the anti-FAM19A5 antibody is linked to an agent, thereby forming an immunoconjugate.
Embodiment 70. The method of any one of Embodiments 27 to 69, wherein the FAM19A5 antagonist is formulated with a pharmaceutically acceptable carrier.
Embodiment 71. The method of Embodiment 70, wherein the FAM19A5 antagonist is administered intravenously, orally, parenterally, intrathecally, intra-cerebroventricularly, pulmonarily, intramuscularly, subcutaneously, intravitreally, or intraventricularly.
Embodiment 72. The method of any one of Embodiments 1 to 71, wherein the subject is a human.

### BRIEF DESCRIPTION OF THE DRAWINGS

FIG. 1 shows a comparison of serum FAM19A5 concentration (ng/ml) among (i) drug-naïve major depressive disorder (DN-MDD) (n=58), (ii) medicated-major depressive disorder (M-MDD) (n=52), (iii) bipolar disorder I (n=26) and II (n=17) (collectively, “BD”), and (iv) heathy control group (HC) (n=112). Each circle represents an individual patient. The horizontal black line shown indicates the average serum FAM19A5 concentration in each of the groups. All comparisons were performed using ANCOVA including age and gender as covariates. “*” above the data points indicates a statistically significant difference (p<0.001) compared to the healthy patients.

FIGS. 2A to 2D provide scatter plots showing the distribution of serum FAM19A5 concentrations (ng/ml) observed in patients from the different groups. FIG. 2A shows the FAM19A5 protein levels for the healthy patients (HC). FIG. 2B shows the FAM19A5 protein levels for the non-medicated major depressive disorder patients (DN-MDD). FIGS. 2C and 2D show the FAM19A5 protein levels for the medicated major depressive disorder patients (M-MDD) and non-medicated bipolar I or II disorder patients (BD), respectively. Each circle represents an individual patient (x-axis). The dotted line indicates a reference value of serum FAM19A5 concentration (i.e., 2 ng/ml).

FIG. 3 provides a table showing the correlation between serum FAM19A5 concentration and the cortical thickness of whole brain in drug-naïve major depressive disorder (n=51), medicated-major depressive disorder (n=51), bipolar disorder (n=28), and heathy control group (n=92). Only cortical regions that showed significant correlation between cortical thickness and serum FAM19A5 concentration are shown. A two-tailed Pearson's partial correlation was performed to analyze the correlations between the cortical thickness and serum FAM19A5 concentration adjusting for age, sex, HDRS score, and medication (only for MDD group) as covariates. Fisher's r to z transformation was performed for the comparison of the correlation coefficients of drug-naïve MDD (DN-MDD), medicated-MDD (M-MDD) or bipolar disorder (BD) groups with those of healthy control (HC) group. The false discovery rate (FDR) was applied in the correlation analyses for multiple comparison correction, q<0.05; 76 comparisons in both hemispheres. The false discovery rate (FDR) was applied in the comparisons of the coefficients for multiple comparison correction, q<0.05; 15 comparisons in drug-naïve MDD group.

FIGS. 4A to 4F provide scatter plots showing the relationship between cortical thickness (y-axis) and serum FAM19A5 protein concentrations (x-axis) from healthy patients (HC, dark gray) or non-medicated major depressive disorder patients (DN-MDD, light gray). The cortical thickness of six different brain regions are shown: L transverse frontopolar gyrus (FIG. 4A), L dorsal posterior cingulate gyrus (FIG. 4B), L pars opercularis (FIG. 4C), L pars triangularis (FIG. 4D), L middle frontal gyrus (FIG. 4E), and R pars opercularis (FIG. 4F). Pearson's partial correlation analyses were performed adjusting for age, sex, and HDRS score (not for HC group). For comparison of correlation coefficients between MDD and healthy control groups, Fisher's r to z transformation was used. In FIGS. 4A to 4F, each circle represents an individual patient and the horizontal lines show the best fit lines.

FIGS. 5A to 5E provide receiver operating characteristic curve (ROC curve) for serum FAM19A5 protein levels from the different comparison groups. The different comparison groups included: (i) drug-naive major depressive disorder patients (DN-MDD) against healthy patients (HC), (FIG. 5A); (ii) medicated major depressive disorder patients (M-MDD) against healthy patients (HC), (FIG. 5B); (iii) non-medicated bipolar I or II disorder patients (BD) against healthy patients (HC), (FIG. 5C); and (iv) non-medicated bipolar I or II disorder patients (BD) against medicated major depressive disorder patients (M-MDD), (FIG. 5D); and (v) non-medicated bipolar I or II disorder patients (BD) against drug-naive major depressive disorder patients (DN-MDD), (FIG. 5E). The AUC, sensitivity, specificity, and accuracy values are provided in the lower right quadrant of each curve.

### DETAILED DESCRIPTION OF THE DISCLOSURE

The present disclosure provides that mood disorders, e.g., major depressive disorder or bipolar disorder, can be diagnosed by measuring the protein level of FAM19A5 or nucleic acid level encoding the FAM19A5 in a sample of the subject (e.g., serum). The present disclosure also provides that subjects having a mood disorder can be identified by measuring the protein level of FAM19A5 or nucleic acid level encoding the FAM19A5 in a sample of the subject (e.g., serum). The present disclosure further provides methods for treating, controlling, or ameliorating a mood disorder (e.g., major depressive disorder or bipolar disorder) comprising administering an agent, e.g., anti-FAM19A5 antagonist, e.g., an anti-FAM19A5 antibody, to a subject in need thereof.

To facilitate an understanding of the disclosure disclosed herein, a number of terms and phrases are defined. Additional definitions are set forth throughout the detailed description.

### I. Definitions

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, “administering” refers to the physical introduction of a therapeutic agent or a composition comprising a therapeutic agent to a subject, using any of the various methods and delivery systems known to those skilled in the art. The different routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “therapeutically effective amount” as used herein refers to an amount of a drug, alone or in combination with another therapeutic agent, effective to “treat” a mood disorder in a subject or reduce the risk, potential, possibility or occurrence of a disease or disorder (e.g., a mood disorder, such as a major depressive disorder or a bipolar disorder). A “therapeutically effective amount” includes an amount of a drug or a therapeutic agent that provides some improvement or benefit to a subject having or at risk of having a disease or disorder. Thus, a “therapeutically effective” amount is an amount that reduces the risk, potential, possibility or occurrence of a disease or disorder or provides some alleviation, mitigation, and/or reduces at least one indicator, and/or decrease in at least one clinical symptom of a disease or disorder (e.g., a mood disorder, such as a major depressive disorder or a bipolar disorder, disclosed herein).

The term “diagnosis” as used herein refers to methods that can be used to determine or predict whether a patient is suffering from a given disease or condition, thereby identifying a subject who is suitable for a treatment. A skilled artisan can make a diagnosis on the basis of one or more diagnostic marker (e.g., FAM19A5), where the presence, absence, amount, or change in amount of the diagnostic marker is indicative of the presence, severity, or absence of the condition. In some embodiments, an increase in FAM19A5 expression, in a biological sample from a subject, is indicative of a mood disorder (e.g., major depressive disorder or bipolar disorder). The term “diagnosis” does not refer to the ability to determine the presence or absence of a particular disease or disorder with 100% accuracy, or even that a given course or outcome is more likely to occur than not. Instead, the skilled artisan will understand that the term “diagnosis” refers to an increased probability that a certain disease or disorder is present in the subject. In some embodiments, the term “diagnosis” includes one or more diagnostic methods of identifying a subject who has a mood disorder.

The composition for diagnosing a mood disorder (e.g., major depressive disorder or bipolar disorder) includes an agent for measuring the protein level of FAM19A5 or the nucleic acid (e.g., mRNA) level encoding FAM19A5 in a sample of a subject in need thereof (e.g., suspected of having a mood disorder). Such agents include oligonucleotides having a sequence complementary to FAM19A5 mRNA, a primer, or a nucleic acid probe that specifically binds to FAM19A5 mRNA, and antibodies, or antigen-binding fragments thereof, that specifically bind to the FAM19A5 protein.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

The term “mood disorder” refers to any type of mental illnesses that affect an individual's emotional state. The mood disorder, as used herein, can be characterized by pervasive, prolonged, and disabling exaggerations of mood and affect that are associated with behavioral, physiologic, cognitive, neurochemical and psychomotor dysfunctions. The mood disorder can be associated with a persistent elevated mood (mania), a persistent depressed mood, or a mood which cycles between mania and depression. The mood disorder can be hereditary in nature and/or induced by a secondary factor (e.g., illness, drugs, medication). Examples of mood disorders include, but are not limited to, major depressive disorder (MDD), bipolar disorder (BD), minor depressive disorder, persistent depressive disorder (dysthymia), seasonable affective disorder (SAD), psychotic depression, postpartum depression, brief recurrent depression, premenstrual dysphoria (PMDD), situational depression, atypical depression, anxiety disorder, and cyclothymic disorder.

The term “major depressive disorder” or “MDD,” as used herein, refers to a mood disorder that is characterized by two or more major depressive episodes. Symptoms of MDD can include fatigue, feelings of worthlessness or guilt, impaired concentration or indecisiveness, insomnia or hypersomnia, markedly diminished interest or pleasure in almost all activities, restlessness, recurring thoughts of death or suicide, and significant weight loss or gain (5% weight change). Diagnostic criteria for MDD, along with other mood disorders, can be found, for example, in the Diagnostic and Statistical Manual of Mental Disorders, fourth edition, DSM-VI®-TR, American Psychiatric Association (DSM IV) and are useful for assessing a subject.

The term “bipolar disorder” refers to a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM IV. Categories of bipolar disorders include, but are not limited to, bipolar disorder I (mania with or without major depression) and bipolar disorder II (hypomania with major depression).

The term “mood,” as used herein, refers to an internal emotional state of a person.

As used herein, the term “mania” or “manic” refers to a disordered mental state of extreme excitement. The term “hypomania” refers to a less extreme manic episode, with lower grade of severity.

The term “family with sequence similarity 19, member A5” or “FAM19A5” refers to a protein that belongs to the TAFA family (also known as FAM19 family) of five highly homologous proteins and is predominantly expressed in brain and the spinal cord. Tang T. Y. et al., Genomics 83(4):727-34 (2004). These proteins contain conserved cysteine residues at fixed positions, and are distantly related to macrophage inflammatory protein 1-alpha (MIP-1-alpha), a member of the CC-chemokine family. The TAFA proteins are predominantly expressed in specific regions of the brain and the spinal cord. These proteins are believed to be generated and secreted by adult neural stem cells in neurogenesis processes. FAM19A5 is also known as TAFA5 or Chemokine-like protein TAFA-5.

FAM19A5 is predominantly expressed in the brain of vertebrates and it is believed that FAM19A5 is important in for the development, differentiation, formation of a complete central nervous system. FAM19A5 also plays a significant role in the pathogenesis of many central nervous system damage and/or degenerative brain diseases (e.g., Huntington's disease, Parkinson's disease, Alzheimer's disease, cerebrospinal damage, strokes, and brain tumors). Upon damage to the central nervous system, neural stem cells produce FAM19A5, which induces the differentiation of normal astrocytes into reactive astrocytes. These reactive astrocytes (along with microglia) can express a wide array of ECMs (e.g., proteoglycans) and induce the formation of glial scars, which can surround the damaged region of the central nervous system like a net and prevent the regeneration of the neurons. Antibodies against FAM19A5 can be used in the prevention and/or treatment of central nervous system injuries and/or diseases. See U.S. Pat. No. 9,579,398.

In humans, the gene encoding FAM19A5 is located on chromosome 22. There are three human FAM19A5 (UniProt: Q7Z5A7) isoforms, which are believed to be produced by alternative splicing: isoform 1 (UniProt: Q7Z5A7-1), which consists of 132 amino acids; isoform 2 (UniProt: Q7Z5A7-2), which consists of 125 amino acids; and isoform 3 (UniProt: Q7Z5A7-3), which consists of 53 amino acids. Human FAM19A5 protein is believed to exist as both membrane bound and soluble (secreted) forms. Isoform 1 is believed to be a membrane protein with one transmembrane region. Isoform 2, which was reported in Tang T. Y. et al., Genomics 83(4):727-34 (2004) as a secreted protein (soluble), contains a signal peptide at amino acid positions 1-25. Isoform 3 is predicted based on EST data. Below are the amino acid sequences of the three known human FAM19A5 isoforms.

(I) Isoform 1 (UniProt: Q7Z5A7-1, transmembrane
protein): this isoform has been chosen as the
canonical sequence.
(SEQ ID NO: 1)
MAPSPRTGSRQDATALPSMSSTFWAFMILASLLIAYCSQLAAGTCEI
VTLDRDSSQPRRTIARQTARCACRKGQIAGTTRARPACVDARIIKTK
QWCDMLPCLEGEGCDLLINRSGWTCTQPGGRIKTTTVS
(II) Isoform 2 (UniProt: Q7Z5A7-2, soluble
protein):
(SEQ ID NO: 2)
MQLLKALWALAGAALCCFLVLVIHAQFLKEGQLAAGTCEIVTLDRDS
SQPRRTIARQTARCACRKGQIAGTTRARPACVDARIIKTKQWCDMLP
CLEGEGCDLLINRSGWTCTQPGGRIKTTTVS
(III) Isoform 3 (UniProt: Q7Z5A7-3):
(SEQ ID NO: 3)
MYHHREWPARIIKTKQWCDMLPCLEGEGCDLLINRSGWTCTQPGGRI
KTTTVS

The term “FAM19A5” includes any variants or isoforms of FAM19A5 which are naturally expressed by cells. Accordingly, antibodies described herein can cross-react with different isoforms in the same species (e.g., different isoforms of human FAM19A5), or cross-react with FAM19A5 from species other than human (e.g., mouse FAM19A5). Alternatively, the antibodies can be specific for human FAM19A5 and cannot exhibit any cross-reactivity with other species. FAM19A5, or any variants and isoforms thereof, can either be isolated from cells or tissues which naturally express them or be recombinantly produced. The polynucleotide encoding human FAM19A5 has the GenBank Accession No. BC039396 and the following sequence:

TABLE 1A
Polynucleotide sequence of human FAM19A5

| | Polynucleotide sequence |
|---|---|
| | (SEQ ID NO: 4) |
| FAM19A5 | ggcggcggag gatggcgcgc gcggggcccg |
| (GenBank | |
| Accession No. | cacgtggagg ccggcgcggg ggcgcgggca |
| BC039396) | |
| | gggccggctg ctgagacgcg ctgctgcccc |
| | ccgcgcgggc gccgcggctt caatggcgcc |
| | atcgcccagg accggcagcc ggcaagatgc |
| | gaccgccctg cccagcatgt cctcaacttt |
| | ctgggcgttc atgatcctgg ccagcctgct |
| | catcgcctac tgcagtcagc tggccgccgg |
| | cacctgtgag attgtgacct tggaccggga |
| | cagcagccag cctcggagga cgatcgcccg |
| | gcagaccgcc cgctgtgcgt gtagaaaggg |
| | gcagatcgcc ggcaccacga gagcccggcc |
| | cgcctgtgtg gacgcaagaa tcatcaagac |
| | caagcagtgg tgtgacatgc ttccgtgtct |
| | ggagggggaa ggctgcgact tgttaatcaa |
| | ccggtcaggc tggacgtgca cgcagcccgg |
| | cgggaggata aagaccacca cggtctcctg |
| | acaaacacag cccctgaggg ggccccggga |
| | gtggccttgg ctccctggag agcccacgtc |
| | tcagccacag ttctccactc gcctcggact |
| | tcacccgttc tctgccgccc gcccactccg |
| | tttccctgtg gtccgtgaag gacggcctca |
| | ggccttggca tcctgagctt cggtctgtcc |
| | agccgacccg aggaggccgg actcagacac |
| | ataggcgggg ggcggcacct ggcatcagca |
| | atacgcagtc tgtgggagcc cggccgcgcc |
| | cagcccccgc cgaccgtggc gttggccctg |
| | ctgtcctcag aggaggagga ggaggaggca |
| | gctccggcag ccacagaagg ctgcagccca |
| | gcccgcctga gacacgacgc ctgccccagg |
| | ggactgtcag gcacagaagc ggcctcctcc |
| | cgtgccccag actgtccgaa ttgcttttat |
| | tttcttatac tttcagtata ctccatagac |
| | caaagagcaa aatctatctg aacctggacg |
| | caccctcact gtcagggtcc ctggggtcgc |
| | ttgtgcgggc gggagggcaa tggtggcaga |
| | gacatgctgg tggccccggc ggagcggaga |
| | gggcggccgt ggtggaggcc tccaccccag |
| | gagcaccccg cacaccctcg gaggacgggc |
| | ttcggctgcg cggaggccgt ggcacacctg |
| | cgggaggcag cgacggcccc cacgcagacg |
| | ccgggaacgc aggccgcttt attcctctgt |
| | acttagatca acttgaccgt actaaaatcc |
| | ctttctgttt taaccagtta aacatgcctc |
| | ttctacagct ccatttttga tagttggata |
| | atccagtatc tgccaagagc atgttgggtc |
| | tcccgtgact gctgcctcat cgatacccca |
| | tttagctcca gaaagcaaag aaaactcgag |
| | taacacttgt ttgaaagaga tcattaaatg |
| | tattttgcaa agcccaaaaa aaaaaaaaaa |
| | a |

The term “antagonist against a FAM19A5 protein” or “FAM19A5 antagonist” refers to all antagonists that suppress the expression of the FAM19A5 protein. Such antagonist can be a peptide, nucleic acid, or a compound. More specifically, the antagonist can be an antisense-oligonucleotide, siRNA, shRNA, miRNA, dsRNA, aptamer, PNA (peptide nucleic acid) targeting FAM19A5, or a vector including the same. In some embodiments, the antagonist can be an antibody, or antigen-binding fragment thereof, that specifically binds to the FAM19A5 protein.

The terms “antibody” and “antibodies” are terms of art and can be used interchangeably herein and refer to a molecule with an antigen binding site that specifically binds an antigen. The terms as used herein include whole antibodies and any antigen binding fragments (i.e., “antigen-binding fragments”) or single chains thereof. An “antibody” refers, in one embodiment, to a glycoprotein comprising at least two heavy (H) chains and two light (L) chains inter-connected by disulfide bonds, or an antigen-binding fragment thereof. In another embodiment, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2 and CH3. In certain naturally-occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “Kabat numbering” and like terms are recognized in the art and refer to a system of numbering amino acid residues in the heavy and light chain variable regions of an antibody, or antigen-binding fragment thereof. In certain aspects, the CDRs of an antibody can be determined according to the Kabat numbering system (see, e.g., Kabat E A & Wu T T (1971) Ann NY Acad Sci 190: 382-391 and Kabat E A et al, (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

The phrases “amino acid position numbering as in Kabat,” “Kabat position,” and grammatical variants thereof refer to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82. See TABLE 1B.

TABLE 1B

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See, e.g., Lefranc, M. P. et al., Dev. Comp. Immunol. 27: 55-77(2003), which is herein incorporated by reference. The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

For all heavy chain constant region amino acid positions discussed in the present disclosure, numbering is according to the EU index first described in Edelman et al., 1969, Proc. Natl. Acad. Sci. USA 63(1):78-85, describing the amino acid sequence of myeloma protein EU, which is the first human IgG1 sequenced. The EU index of Edelman et al. is also set forth in Kabat et al., 1991, Sequences of Proteins of Immunological Interest, 5th Ed., United States Public Health Service, National Institutes of Health, Bethesda. Thus, the phrases “EU index as set forth in Kabat” or “EU index of Kabat” and “position . . . according to the EU index as set forth in Kabat,” and grammatical variants thereof refer to the residue numbering system based on the human IgG1 EU antibody of Edelman et al. as set forth in Kabat 1991.

The numbering system used for the variable domains (both heavy chain and light chain) and light chain constant region amino acid sequence is that set forth in Kabat 1991.

Antibodies can be of any type (e.g., IgG, IgE, IgM, IgD, IgA, or IgY), any class (e.g., IgD, IgG2, IgG3, IgG4, IgA1, or IgA2), or any subclass (e.g., IgG1, IgG2, IgG3, and IgG4 in humans; and IgG1, IgG2a, IgG2b, and IgG3 in mice) of immunoglobulin molecule. Immunoglobulins, e.g., IgG1, exist in several allotypes, which differ from each other in at most a few amino acids. An antibody disclosed herein can be from any of the commonly known isotypes, classes, subclasses, or allotypes. In certain embodiments, the antibodies described herein are of the IgG1, IgG2, IgG3, or IgG4 subclass or any hybrid thereof. In certain embodiments, the antibodies are of the human IgG1 subclass or the human IgG2 or human IgG4 subclass.

“Antibody” includes, by way of example, both naturally-occurring and non-naturally-occurring antibodies; monoclonal and polyclonal antibodies; chimeric and humanized antibodies; human and non-human antibodies; wholly synthetic antibodies; single chain antibodies; monospecific antibodies; multispecific antibodies (including bispecific antibodies); tetrameric antibodies comprising two heavy chain and two light chain molecules; an antibody light chain monomer; an antibody heavy chain monomer; an antibody light chain dimer, an antibody heavy chain dimer; an antibody light chain-antibody heavy chain pair; intrabodies; heteroconjugate antibodies; monovalent antibodies; camelized antibodies; affybodies; anti-idiotypic (anti-Id) antibodies (including, e.g., anti-anti-Id antibodies), and single-domain antibodies (sdAbs), which include binding molecules consisting of a single monomeric variable antibody domain that are fully capable of antigen binding (e.g., a VH domain or a VL domain). Harmen M. M. and Hard H. J. Appl Microbiol Biotechnol. 77(1): 13-22 (2007)).

The terms “antigen-binding portion” and “antigen-binding fragment” of an antibody are used interchangeably and refer to one or more fragments of an antibody that retain the ability to specifically bind to an antigen (e.g., human FAM19A5). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody, e.g., an anti-FAM19A5 antibody described herein, include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, and disulfide-linked Fvs (sdFv); (v) a dAb fragment (Ward et al., Nature 341:544-546 (1989)), which consists of a VH domain; and (vi) an isolated complementarity determining region (CDR) or (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 242:423-426 (1988); and Huston et al., Proc. Natl. Acad. Sci. USA 85:5879-5883 (1988). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR).

Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In some embodiments, the variable region is a primate (e.g., non-human primate) variable region. In other embodiments, the variable region comprises rodent or murine CDRs and primate (e.g., non-human primate) framework regions (FRs).

As used herein, the term “heavy chain” (HC) when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” (LC) when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

The terms “VL” and “VL domain” are used interchangeably to refer to the light chain variable region of an antibody.

The terms “VH” and “VH domain” are used interchangeably to refer to the heavy chain variable region of an antibody.

As used herein, the terms “constant region” and “constant domain” are interchangeable and are known in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxy-terminus of the heavy chain, wherein the numbering is according to the EU index as in Kabat. The CH2 domain of a human IgG Fc region extends from about amino acid 231 to about amino acid 340, whereas the CH3 domain is positioned on C-terminal side of a Cm domain in an Fc region, i.e., it extends from about amino acid 341 to about amino acid 447 of an IgG. As used herein, the Fc region can be a native sequence Fc, including any allotypic variant, or a variant Fc (e.g., a non-naturally-occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG Fc region; native sequence human IgG2 Fc region; native sequence human IgG3 Fc region; and native sequence human IgG4 Fc region as well as naturally-occurring variants thereof. Native sequence Fc includes the various allotypes of Fes (see, e.g., Jefferis et al., mAbs 1:1 (2009); Vidarsson G. et al. Front Immunol. 5:520 (published online Oct. 20, 2014)).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Human IgG1 binds to most human Fc receptors and elicits the strongest Fc effector functions. It is considered equivalent to murine IgG2a with respect to the types of activating Fc receptors that it binds to. Conversely, human IgG4 elicits the least Fc effector functions. Vidarsson G. et al. Front Immunol. 5:520 (published online Oct. 20, 2014).

The constant region can be manipulated, e.g., by recombinant technology, to eliminate one or more effector functions. An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with a binding domain (e.g., an antibody variable domain). Accordingly, the term “a constant region without the Fc function” include constant regions with reduced or without one or more effector functions mediated by Fc region.

Effector functions of an antibody can be reduced or avoided by different approaches. Effector functions of an antibody can be reduced or avoided by using antibody fragments lacking the Fc region (e.g., such as a Fab, F(ab′)₂, single chain Fv (scFv), or a sdAb consisting of a monomeric VH or VL domain). Alternatively, the so-called aglycosylated antibodies can be generated by removing sugars that are linked to particular residues in the Fc region to reduce the effector functions of an antibody while retaining other valuable attributes of the Fc region (e.g., prolonged half-life and heterodimerization). Aglycosylated antibodies can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells). See e.g., U.S. Pub. No. 20120100140. Another approach is to employ Fc regions from an IgG subclass that have reduced effector function, for example, IgG2 and IgG4 antibodies are characterized by having lower levels of Fc effector functions than IgG1 and IgG3. The residues most proximal to the hinge region in the CH2 domain of the Fc part are responsible for effector functions of antibodies as it contains a largely overlapping binding site for C1q (complement) and IgG-Fc receptors (FcγR) on effector cells of the innate immune system. Vidarsson G. et al. Front Immunol. 5:520 (published online Oct. 20, 2014). Accordingly, antibodies with reduced or without Fc effector functions can be prepared by generating, e.g., a chimeric Fc region which comprises a CH2 domain from an IgG antibody of the IgG4 isotype and a CH3 domain from an IgG antibody of the IgG1 isotype, or a chimeric Fc region which comprises hinge region from IgG2 and CH2 region from IgG4 (see, e.g., Lau C. et al. J. Immunol. 191:4769-4777 (2013)), or an Fc region with mutations that result in altered Fc effector functions, e.g., reduced or no Fc functions. Such Fc regions with mutations are known in the art. See, e.g., U.S. Pub. No. 20120100140 and U.S. and PCT applications cited therein; and An et al., mAbs 1:6, 572-579 (2009).

A “hinge”, “hinge domain” or “hinge region” or “antibody hinge region” refers to the domain of a heavy chain constant region that joins the CH1 domain to the CH2 domain and includes the upper, middle, and lower portions of the hinge (Roux et al., J Immunol. 161:4083 (1998)). The hinge provides varying levels of flexibility between the binding and effector regions of an antibody and also provides sites for intermolecular disulfide bonding between the two heavy chain constant regions. As used herein, a hinge starts at Glu216 and ends at Gly237 for all IgG isotypes (Roux et al., J Immunol 161:4083 (1988)). The sequences of wild-type IgG1, IgG2, IgG3 and IgG4 hinges are known in the art. See e.g., Kabat E A et al., (1991) Sequences of Proteins of Immunological Interest, Fifth Edition, U.S. Department of Health and Human Services, NIH Publication No. 91-3242; Vidarsson G. et al. Front Immunol. 5:520 (published online Oct. 20, 2014).

The term “CH1 domain” refers to the heavy chain constant region linking the variable domain to the hinge in a heavy chain constant domain. As used herein, a CH1 domain starts at A118 and ends at V215. The term “CH1 domain” includes wildtype CH1 domains, as well as naturally existing variants thereof (e.g., allotypes). CH1 domain sequences of IgG1, IgG2, IgG3, and IgG4 (including wildtype and allotypes) are known in the art. See e.g., Kabat E A et al., (1991) supra and Vidarsson G. et al. Front Immunol. 5:520 (published online Oct. 20, 2014). Exemplary CH1 domains include CH1 domains with mutations that modify a biological activity of an antibody, e.g., half-life, e.g., described in U.S. Pub. No. 20120100140 and U.S. patents and publications and PCT publications cited therein.

The term “CH2 domain” refers to the heavy chain constant region linking the hinge to the CH3 domain in a heavy chain constant domain. As used herein, a CH2 domain starts at P238 and ends at K340. The term “CH2 domain” includes wildtype CH2 domains, as well as naturally existing variants thereof (e.g., allotypes). CH2 domain sequences of IgG1, IgG2, IgG3, and IgG4 (including wildtype and allotypes) are known in the art. See e.g., Kabat E A et al., (1991) supra and Vidarsson G. et al. Front Immunol. 5:520 (published online Oct. 20, 2014). Exemplary CH2 domains include CH2 domains with mutations that modify a biological activity of an antibody, e.g., half-life and/or reduced Fc effector function, e.g., described in U.S. Pub. No. 20120100140 and U.S. patents and publications and PCT publications cited therein.

The term “CH3 domain” refers to the heavy chain constant region that is C-terminal to the CH2 domain in a heavy chain constant domain. As used herein, a CH3 domain starts at G341 and ends at K447. The term “CH3 domain” includes wildtype CH3 domains, as well as naturally existing variants thereof (e.g., allotypes). CH3 domain sequences of IgG1, IgG2, IgG3, and IgG4 (including wildtype and allotypes) are known in the art. See e.g., Kabat E A et al., (1991) supra and Vidarsson G. et al. Front Immunol. 5:520 (published online Oct. 20, 2014). Exemplary CH3 domains include CH3 domains with mutations that modify a biological activity of an antibody, e.g., half-life, e.g., described in U.S. Pub. No. 20120100140 and U.S. patents and publications and PCT publications cited therein.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally-occurring variants within a specific isotype group, which variants differ in a few amino acids (see, e.g., Jefferis et al. (2009) mAbs 1:1). Antibodies described herein can be of any allotype. Allotypes of IgG1, IgG2, IgG3, and IgG4 are known in the art. See e.g., Kabat E A et al., (1991) supra; Vidarsson G. et al. Front Immunol. 5:520 (published online Oct. 20, 2014); and Lefranc M P, mAbs 1:4, 1-7(2009).

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other antibodies having different antigenic specificities (e.g., an isolated antibody that specifically binds to FAM19A5 is substantially free of antibodies that specifically bind antigens other than FAM19A5). An isolated antibody that specifically binds to an epitope of FAM19A5 can, however, have cross-reactivity to other FAM19A5 proteins from different species.

“Binding affinity” generally refers to the strength of the sum total of interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (KA). The K_{D} is calculated from the quotient of k_{off}/kₒₙ and is expressed as a molar concentration (M), whereas KA is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE™ or kinetic exclusion assay (KINEXA®).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE™, KINEXA® 3000 instrument (Sapidyne Instruments, Boise, ID), or other assays known in the art. In a specific embodiment, molecules that specifically bind to an antigen bind to the antigen with a KA that is at least 2 logs, 2.5 logs, 3 logs, 4 logs or greater than the KA when the molecules bind to another antigen.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 10⁻⁹ M or less, and most preferably between 10⁻⁸ M and 10⁻¹⁰ M or less, when determined by, e.g., immunoassays (e.g., ELISA) or surface plasmon resonance (SPR) technology in a BIACORE™ 2000 instrument using the predetermined antigen, but does not bind with high affinity to unrelated antigens.

As used herein, the term “antigen” refers to any natural or synthetic immunogenic substance, such as a protein, peptide, or hapten. An antigen can be FAM19A5 or a fragment thereof.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 1, 2, 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from FMAM19A5) are tested for reactivity with a given antibody (e.g., anti-FAM19A5 antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., Acta Crystallogr D Biol Crystallogr 50(Pt 4): 339-350 (1994); McPherson A Eur J Biochem 189: 1-23 (1990); Chayen N E Structure 5: 1269-1274 (1997); McPherson A J Biol Chem 251:6300-6303 (1976)). Antibody:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al., U.S. 2004/0014194), and BUSTER (Bricogne G Acta Crystallogr D Biol Crystallogr 49(Pt 1): 37-60 (1993); Bricogne G Meth Enzymol 276A: 361-423 (1997), ed Carter C W; Roversi P et al., Acta Crystallogr D Biol Crystallogr 56(Pt 10): 1316-1323 (2000)). Mutagenesis mapping studies can be accomplished using any method known to one of skill in the art. See, e.g., Champe M et al., J Biol Chem 270 (1995): 1388-1394 and Cunningham B C & Wells J A Science 244: 1081-1085 (1989) for a description of mutagenesis techniques, including alanine scanning mutagenesis techniques.

The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on FAM19A5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “monoclonal antibody,” as used herein, refers to an antibody that displays a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg Nature Biotech. 23(9): 1117-1125 (2005)), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

The term “cross-reacts,” as used herein, refers to the ability of an antibody described herein to bind to FAM19A5 from a different species. For example, an antibody described herein that binds human FAM19A5 can also bind another species of FAM19A5 (e.g., mouse FAM19A5). As used herein, cross-reactivity can be measured by detecting a specific reactivity with purified antigen in binding assays (e.g., SPR, ELISA) or binding to, or otherwise functionally interacting with, cells physiologically expressing FAM19A5. Methods for determining cross-reactivity include standard binding assays as described herein, for example, by BIACORE™ surface plasmon resonance (SPR) analysis using a BIACORE™ 2000 SPR instrument (Biacore AB, Uppsala, Sweden), or flow cytometric techniques.

The term “naturally-occurring” as applied to an object herein refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

The term “therapeutically effective amount” as used herein refers to an amount of a drug, alone or in combination with another therapeutic agent, effective to “treat” a mood disorder in a subject or reduce the risk, potential, possibility or occurrence of a mood disorder (e.g., major depressive disorder or bipolar disorder). A “therapeutically effective amount” includes an amount of a drug or a therapeutic agent that provides some improvement or benefit to a subject having or at risk of having a mood disorder. Thus, a “therapeutically effective” amount is an amount that reduces the risk, potential, possibility or occurrence of a mood disorder or provides alleviation, mitigation, and/or reduces at least one indicator, and/or decrease in at least one clinical symptom of a mood disorder (e.g., major depressive disorder or bipolar disorder).

### II. Method of Diagnosing Mood Disorders

Disclosed herein are methods of diagnosing a mood disorder (e.g., major depressive disorder or bipolar disorder) in a subject in need of comprising contacting an FAM19A5 antagonist (e.g., an anti-FAM19A5 antibody, or antigen-binding fragment thereof) with a biological sample of the subject and measuring a protein level of FAM19A5 or a nucleic acid (e.g., an mRNA) level encoding FAM19A5 in the sample. In some embodiments, the mood disorder is selected from the group consisting of a major depressive disorder (MDD), bipolar disorder (BD), persistent depressive disorder, seasonal affective disorder (SAD), psychotic depression, postpartum depression, premenstrual dysphoric disorder (PMDD), situational depression, atypical depression, and any combination thereof. In certain embodiments, the mood disorder is a major depressive disorder. In other embodiments, the mood disorder is a bipolar disorder.

In one embodiment, the biological sample has at least 1.1 fold, at least 1.2 fold, at least 1.3 fold, at least 1.4 fold, at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 15 fold, at least 20 fold, at least 25 fold, or at least 30 fold increase in the protein level of FAM19A5 or the nucleic acid level encoding FAM19A5 compared to the protein level of FAM19A5 or the nucleic acid level encoding FAM19A5 in a reference sample (e.g., sample of a subject who does not have a mood disorder). The subject who exhibits an increased protein level of FAM19A5 or an increased nucleic acid level encoding FAM19A5 can be identified as a subject suspected of a mood disorder.

In one embodiment, the reference sample can be obtained from the same subject wherein the reference sample was obtained prior to the initiation of the mood disorder. In another embodiment, the reference sample can be obtained from a control subject or a population of control subjects who do not have a mood disorder, e.g., health control subjects.

In certain embodiments, the reference protein level of FAM19A5 is about 2.0 ng/ml, about 1.9 ng/ml, about 1.8 ng/ml, about 1.7 ng/ml, about 1.6 ng/ml, about 1.5 ng/ml, about 1.4 ng/ml, about 1.3 ng/ml, about 1.2 ng/ml, about 1.1 ng/ml, about 1 ng/ml, about 0.9 ng/ml, about 0.8 ng/ml, about 0.7 ng/ml, about 0.6 ng/ml, about 0.5 ng/ml, about 0.4 ng/ml, about 0.3 ng/ml, about 0.2 ng/ml, about 0.1 ng/ml, or lower as measured by an ELISA. In other embodiments, the reference protein level of FAM19A5 is about 2.0 ng/ml or lower as measured by an ELISA.

In some embodiments, the subject suspected of having a mood disorder shows a FAM19A5 protein level of at least 2.1 ng/ml, at least 2.2 ng/ml, at least 2.3 ng/ml, at least 2.4 ng/ml, at least 2.5 ng/ml, at least 2.6 ng/ml, at least 2.7 ng/ml, at least 2.8 ng/ml, at least 2.9 ng/ml, at least 3.0 ng/ml, at least 3.5 ng/ml, at least 4.0 ng/ml, at least 4.5 ng/ml, at least 5.0 ng/ml, at least 6.0 ng/ml, at least 7 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, or at least 25 ng/ml. In other embodiments, the subject suspected of having a mood disorder shows a FAM19A5 protein level of about 2.0 ng/ml to about 1 μg/ml, 10 ng/ml to about 1 μg/ml, 2 ng/ml to about 500 ng/ml (or about 400 ng/ml, 300 ng/ml, 200 ng/ml, or 100 ng/ml), 3 ng/ml to about 400 ng/ml,

In connection with the current disclosure, it has been shown that subjects with a mood disorder express higher level of FAM19A5 in the peripheral blood. See Example 1. Accordingly, the method disclosed herein can be used to diagnose a mood disorder (e.g., major depressive disorder or bipolar disorder) in a subject by measuring the subject's protein level of FAM19A5 or nucleic acid level encoding FAM19A5 in the serum, where an increased level as compared to a reference level (e.g., FAM19A5 level in a subject without a mood disorder) would suggest that the subject has a mood disorder. In some embodiments, the present disclosure can be used to confirm the presence of a mood disorder by measuring the FAM19A5 protein level in a subject who is suspected of having a mood disorder. In other embodiments, the present disclosure can be used to identify a subject having a mood disorder. Such methods can be more precise and quantitative than the currently available diagnostic methods (e.g., healthcare professional's subjective assessment of a patient's behavior).

In other embodiments, the disclosure includes a method of identifying a subject who needs a treatment for a mood disorder comprising measuring the protein or nucleic acid level of FAM19A5, wherein an increased level of the FAM19A5 protein or nucleic acid encoding FAM19A5 compared to a control level indicates that the subject has a mood disorder. In some embodiments, the disclosure further comprises instructing a healthcare provider to treat the subject who is identified as having a mood disorder. In other embodiments, the disclosure further comprises informing the healthcare provider about the diagnosis, e.g., presence of the mood disorder. In some embodiments, the disclosure further comprises administering one or more agent to treat the mood disorder.

In some embodiments, a method of the present disclosure includes a method of measuring cortical thickness of the subject who is suspected of having a mood disorder. As shown herein, the present application shows that the cortical thickness has inverse relationship to the level of FAM19A5 protein. Therefore, in one embodiment, the present disclosure includes a method of measuring cortical thickness in addition to the FAM19A5 protein or nucleic acid level to identify a subject who has a mood disorder, wherein reduced cortical thickness indicates that the subject has a mood disorder.

In certain embodiments, the cortical thickness of the potential subject is reduced at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% compared to the cortical thickness of the control subjects, e.g., healthy subject.

In some embodiments, the cortical region that can be measured for the method includes pars opercularis, planum temporale, left transverse frontopolar and dorsal posterior cingulate gyrus, pars triangularis, middle frontal gyrus, and right lingual and/or anterior transverse temporal gyrus. In some embodiments, the cortical thickness is measured by MRI, e.g., high resolution MRI optionally with powerful image processing techniques known in the art. In other embodiments, the cortical thickness is measured by 3.0 T Siemens Trio whole-body imaging system (Siemens Medical Systems, Iselin, New Jersey, United States).

In one embodiment, the protein level of FAM19A5 is measured by an immunohistochemistry, a Western blotting, a radioimmunoassay, an enzyme linked immunosorbent assay (ELISA), a radioimmunodiffusion, an immunoprecipitation assay, an Ouchterlony immunodiffusion method, a rocket immunoelectrophoresis, a tissue immunostaining method, a complement fixation assay, FACS, or a protein chip. In one embodiment, the nucleic acid level encoding FAM19A5 protein is measured by a RT-PCR, a real time polymerase chain reaction, or a Northern blot. In some embodiments, the biological sample comprises a tissue, cell, blood, serum, plasma, saliva, cerebro spinal fluid, intravitreal fluid, or urine.

In some embodiments, the protein level of FAM19A5 is measured by an assay using an anti-FAM19A5 antibody. In other embodiments, the anti-FAM19A5 antibody is an antibody disclosed herein, e.g., 2-13, 3-2, 1-65, 1-28, or any combination thereof.

In other embodiments, the diagnosis of the present disclosure can be combined with one or more known diagnostic methods for mood disorders. In one embodiment, one or more diagnostic tests can be used to rule out other serious medical conditions that can cause similar symptoms of mood disorders, e.g., thyroid disease, vitamin D deficiency, central nervous system tumors, head trauma, multiple sclerosis, stroke, syphilis, various cancers (pancreas, prostate, breast), and/or another medical problem. In a situation where the subject has other serious medical conditions, the subject can be treated for the other serious medical conditions only without the treatment for mood disorders. In other embodiments, the subject can be treated for the other serious medical conditions along with the mood disorders.

In other embodiments, the one or more diagnostic tests comprises a physical exam. The physical exam can be used to rule out a physical cause for depression, e.g., hypothyroidism, hyperthyroidism, Cushing's disease.

In addition to the FAM19A5 protein or nucleic acid levels, in some embodiments, the methods of the present disclosure also comprises a blood test to check for anemia as well as thyroid, other hormone, calcium and/or vitamin D levels, electrolytes, liver function, toxicology screening, and/or kidney function. In other embodiments, the test can also include CT scan or MRI of the brain to identify serious illnesses such as a brain tumor, electrocardiogram (ECG) to diagnose some heart problems, and/or electroencephalogram (EEG) to record electrical activity of the brain.

In other embodiments, one or more diagnostic tests that can be used in combination with the FAM19A5 protein and/or nucleic acid level is a questionnaire. Exemplary questionnaires include: (i) the Patient Health Questionnaire-9 (PHQ-9) (i.e., a 9-item self-administered diagnostic screening and severity tool based on current diagnostic criteria for major depression); (ii) Beck Depression Inventory (BDI) (i.e., a 21-question multiple-choice self-report that measures the severity of depression symptoms and feelings); (iii) Zung Self-Rating Depression Scale (i.e., a short survey that measures the level of depression, ranging from normal to severely depressed), (iv) Center for Epidemiologic Studies-Depression Scale (CES-D) (i.e., an instrument that allows patients to evaluate their feelings, behavior, and outlook from the previous week); (v) Hamilton Rating Scale for Depression (HRSD), also known as the Hamilton Depression Rating Scale (HDRS) or abbreviated to HAM-D (i.e., a multiple choice questionnaire that doctors may use to rate the severity of a patient's depression), or any combination of (i) to (v) thereof.

### III. Methods of Treating Mood Disorders

Disclosed herein are also methods of treating, controlling, ameliorating, or reducing a mood disorder (e.g., major depressive disorder or bipolar disorder) in a subject in need thereof based on the diagnosis disclosed herein. In some embodiments, the treatment of a mood disorder comprises administering to the subject an antagonist against FAM19A5. In one embodiment, the antagonist is an antibody, or antigen-binding fragment thereof, that specifically binds to the FAM19A5 protein (“an anti-FAM19A5 antibody or antigen-binding fragment thereof”), a polynucleotide encoding the anti-FAM19A5 antibody or an antigen binding portion thereof, or a vector comprising the polynucleotide thereof. In some embodiments, the anti-FAM19A5 antibody, or antigen-binding fragment thereof, binds to FAM19A5 protein and reduces FAM19A5 activity. In certain embodiments, the reduced FAM19A5 activity increases the thickness of a cortical gyrus to normal levels (e.g., thickness of a corresponding cortical gyrus in a healthy subject).

In other embodiments, the subject who is diagnosed as having a mood disorder is treated with one or more antidepressant. As used herein, the term “antidepressants” or “antidepressant treatments” refers to any agent or any form of psychological treatment or psychotherapy typically used to treat or alleviate clinical depression. Anti-depressant agents include compounds of different classes including, for example, selective serotonin reuptake inhibitors (e.g., sertraline or escitalopram), tricyclic antidepressants (e.g., desipramine), and dopamine reuptake inhibitors (e.g., bupropion). Typically, antidepressants of different classes exert their therapeutic effects via different biochemical pathways.

Examples of antidepressants include, but are not limited to, selective serotonin reuptake inhibitors (SSRIs), serotonin-norepinephrine reuptake inhibitors (SNRIs), serotonin modulators and stimulators (SMSs), serotonin antagonists and reuptake inhibitors (SARIs), norepinephrine reuptake inhibitors (NRIs), tricyclic antidepressants (TCAs), tetracyclic antidepressants (TeCAs), and monoamine oxidase inhibitors (MAOIs).

Examples of SSRIs include citalopram (CELEXA®), escitalopram (LEXAPRO®, CIPRALEX®), paroxetine (PAXIL®, SEROXAT®), fluoxetine (PROZAC®), fluvoxamine (LUVOX®), sertraline (ZOLOFT®, LUSTRAL®), zimelidine (NORMUD®, ZELMID®), and indalpine (UPSTENE®). Examples of SNRIs include desvenlafaxine (PRISTIQ®), duloxetine (CYMBALTA®), levomilnacipran (FETZIMA®), milnacipran (IXEL®, SAVELLA®), tofenacin (ELAMOL®, TOFACINE®), and venlafaxine (EFFEXOR®). Examples of SMSs include vilazodone (VIIBRYD®) and vortioxetine (TRINTELLIX®). Examples of SARIs include etoperidone (AXIOMIN®, ETONIN®), nefazodone (NEFADAR®, SERZONE®), and trazodone (DESYREL®). Examples of NRIs include reboxetine (EDRONAX®), viloxazine (VIVALAN®), and atomoxetine (STRATTERA®). Examples of TCAs include amitriptyline (ELAVIL®, ENDEP®), amitriptylinoxide (AMIOXID®, AMBIVALON®, EQUILIBRIN®), clomipramine (ANAFRANIL®), desipramine (NORPRAMIN®, PERTOFRANE®), dibenzepin (NOVERIL®, VICTORIL®), dimetacrine (ISTONIL®), dosulepin (PROTHIADEN®), doxepin (ADAPIN®, SINEQUAN®), imipramine (TOFRANIL®), lofepramine (LOMONT®, GAMANIL®), melitracen (DIXERAN®, MELIXERAN®, TRAUSABUN®), nitroxazepine (SINTAMIL®), nortriptyline (PAMELOR®, AVENTYL®), noxiptiline (AGEDAL®, ELRONON®, NOGEDAL®), pipofezine (AZAFEN®/AZAPHEN®), protriptyline (VIVACTIL®), trimipramine (SURMONTIL®), butriptyline (EVADYNE®), demexiptiline (DEPARON®, TINORAN®), imipraminoxide (IMIPREX®, ELEPSIN®), iprindole (PRONDOL®, GALATUR®, TETRAN®), metapramine (TIMAXEL®), propizepine (DEPRESSIN®, VAGRAN®), quinupramine (KINUPRIL®, KEVOPRIL®), opipramol (INSIDON®), tianeptine (STABLON®), amineptine (SURVECTOR®, MANEON®), and tiazesim (ALTINIL®). Examples of TeCAs include amoxapine (ASENDIN®), maprotiline (LUDIOMIL®), mianserin (BOLVIDON®, NORVAL®, TOLVON®), mirtazapine (REMERON®), and setiptiline (TECIPUL®). Examples of MAOIs include isocarboxazid (MARPLAN®), phenelzine (NARDIL®), tranylcypromine (PARNATE®), benmoxin (NEURALEX®), iproclozide (SURSUM®), iproniazid (MARSILID®), mebanazine (ACTOMOL®), nialamide (NIAMID®), octamoxin (XIMAOL®), pheniprazine (CATRON®), phenoxypropazine (DRAZINE®), pivhydrazine (TERSAVID®), safrazine (SAFRA®), Selegiline (ELDEPRYL®, ZELAPAR®, EMSAM®), caroxazone (SURODIL®, TIMOSTENIL®), metralindole (INKAZAN®), moclobemide (AURORIX®, MANERIX®), pirlindole (PIRAZIDOL®), toloxatone (HUMORYL®), eprobemide (BEFOL®), minaprine (BRANTUR®, CANTOR®).

In some embodiments, the subject who is diagnosed as having a mood disorder is treated with one or more antipsychotic agents. As used herein, the term “antipsychotics” or “antipsychotic agents” (also known as neuroleptics or major tranquilizers) refers to a class of medication used to manage psychosis (e.g., delusions, hallucinations, paranoia, or disordered thought). Antipsychotics can be categorized as typical (first-generation or conventional) and atypical (second-generation).

Examples of typical antipsychotics include chlorpromazine (THORAZINE®, LARGACTIL®), chlorprothixene (TARACTAN®), levomepromazine (NOZINAN®, LEVOPROME®), mesoridazine (SERENTIL®), periciazine (NEULEPTIL®), promazine (SPARINE®), thioridazine (MELLARIL®), loxapine (LOXITANE®), molindone (MOBAN®), perphenazine (TRILAFON®), thiothixene (NAVANE®), droperidol (INAPSINE®), flupentixol (DEPIXOL®, FLUANXOL®), fluphenazine (PROLIXIN®), haloperidol (HALDOL®), pimozide (ORAP®), prochlorperazine (COMPAZINE®), thioproperazine (MAJEPTIL®), trifluoperazine (STELAZINE®), and zuclopenthixol (CLOPIXOL®).

Examples of atypical antipsychotics include amisulpride (SOLIAN®), lurasidone (LATUDA®), quetiapine (SEROQUEL®), risperidone (RISPERDAL®), olanzapine (ZYPREXA®), aripiprazole (ABILIFY®), ziprasidone (GEODON®), pimavanserin (NUPLAZID®), clozapine (CLOZARIL®), paliperidone (INVEGA®), asenapine (SAPHRIS®), brexpiprazole (REXULTI®), cariprazine (VRAYLAR®), and iloperidone (FANAPT®).

In other embodiment, the subject who is diagnosed as having a mood disorder according to the present disclosure is treated with a psychotherapy. The term “psychotherapy” includes any of various means involving communication between a patient and a therapist. Antidepressant treatment can also include a combination of antidepressant agents and psychotherapy.

In one embodiment, the subject being treated in the method is a nonhuman animal such as a rat or a mouse. In one embodiment, the subject being treated in the method is a human.

In some embodiments, the FAM19A5 antagonist is administered to the subject intravenously, orally, parenterally, intrathecally, intra-cerebroventricularly, pulmonarily, intramuscularly, subcutaneously, intravitreally, or intraventricularly. For administration of an anti-FAM19A5 antibody or antigen-binding portion thereof, e.g., described herein, the dosage ranges from about 0.0001 to 100 mg/kg.

In some embodiments, an anti-FAM19A5 antibody or antigen-binding portion thereof, or a composition thereof can be administered in combination with one or more additional agent for treating a mood disorder. In some embodiments, the additional agent is an antidepressant agent comprising selective serotonin reuptake inhibitors (SSRIs), serotonin-norepinephrine reuptake inhibitors (SNRIs), serotonin modulators and stimulators (SMSs), serotonin antagonists and reuptake inhibitors (SARIs), norepinephrine reuptake inhibitors (NRIs), tricyclic antidepressants (TCAs), tetracyclic antidepressants (TeCAs), monoamine oxidase inhibitors (MAOIs), or any combination thereof. In some embodiments, the additional agent is an antipsychotic agent (e.g., typical or atypical). Examples of antidepressant agents and antipsychotic agents are disclosed herein and also known in the art. Dose and administration of the one or more additional therapeutic drugs are known in the art, e.g., as instructed by the product label of the respective drug.

### IV. FAM19A5 Antagonists

One or more FAM19A5 antagonists can be used to diagnose a subject who has a mood disorder or to treat the subject. In one embodiment, the FAM19A5 antagonist is an antisense oligonucleotide, siRNA, shRNA, miRNA, dsRNA, aptamer, PNA (peptide nucleic acid) that specifically targets FAM19A5, or a vector including the same. In another embodiment, the FAM19A5 antagonist is an antibody, or an antigen-binding fragment thereof, that specifically binds to the FAM19A5 protein, a polynucleotide encoding the anti-FAM19A5 antibody, or antigen-binding fragment thereof, or a vector comprising the polynucleotide thereof.

Antibodies that are useful in the methods disclosed herein include monoclonal antibodies, which are characterized by particular functional features or properties. For example, the antibodies specifically bind human FAM19A5, including soluble FAM19A5 and membrane bound FAM19A5. In addition to binding specifically to soluble and/or membrane bound human FAM19A5, the antibodies described herein also (a) binds to soluble human FAM19A5 with a K_{D} of 10 nM or less; (b) binds to membrane bound human FAM19A5 with a K_{D} of 10 nM or less; or both (a) and (b).

In some embodiments, the anti-FAM19A5 antibody or antigen binding portion thereof specifically binds to soluble human FAM19A5 or membrane-bound human with high affinity, for example, with a K_{D} of 10⁻⁷ M or less, 10⁻⁸ M or less, 10⁻⁹ M (1 nM) or less, 10⁻¹⁰ M (0.1 nM) or less, 10⁻¹¹ M or less, or 10⁻¹² M (1 pM) or less, e.g., 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, e.g., 10⁻¹² M, 5×10⁻¹² M, 10⁻¹¹ M, 5×10⁻¹¹ M, 10⁻¹⁰ M, 5×10⁻¹⁰ M, 10⁻⁹ M, 5×10⁻⁹ M, 10⁻⁸ M, 5×10⁻⁸ M, 10⁻⁷ M, or 5×10⁻⁷ M. Standard assays to evaluate the binding ability of the antibody toward human FAM19A5 of various species are known in the art, including for example, ELISAs, Western blots, and RIAs. Suitable assays are described in detail in the Examples. The binding kinetics (e.g., binding affinity) of the antibodies also can be assessed by standard assays known in the art, such as by ELISA, BIACORE™ analysis or KINEXA®. Assays to evaluate the effects of the antibodies on functional properties of FAM19A5 (e.g., ligand binding) are described in further detail infra and in the Examples.

In some embodiments, the anti-FAM19A5 antibody, or antigen binding fragment thereof, binds to soluble human FAM19A5 with a K_{D}, e.g., as determined by ELISA, of 10⁻⁷ M or less, 10⁻⁸ M (10 nM) or less, 10⁻⁹ M (1 nM) or less, 10⁻¹⁰ M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, or 10⁻⁸ M to 10⁻⁷ M. In some embodiments, the anti-FAM19A5 antibody, or antigen binding fragment thereof, binds to soluble FAM19A5 with a K_{D} of 10 nM or less, e.g., between 0.1 and 10 nM, between 0.1 and 5 nM, between 0.1 and 1 nM, between 0.5 and 10 nM, between 0.5 and 5 nM, between 0.5 and 1 nM, between 1 and 10 nM, between 1 and 5 nM, or between 5 and 10 nM. In some embodiments, the anti-FAM19A5 antibody or antigen binding portion thereof specifically binds to soluble human FAM19A5 with a K_{D} of about 1 pM, 2 pM, 3 pM, 4 pM, 5 pM, 6 pM, 7 pM, 8 pM, 9 pM, 10 pM, 20 pM, 30 pM, 40 pM, 50 pM, 60 pM, 70 pM, 80 pM, 90 pM, 100 pM, 200 pM, 300 pM, 400 pM, 500 pM, 600 pM, 700 pM, 800 pM, or 900 pM, or about 1 nM, 2 nM, 3 nM, 4 nM, 5 nM, 6 nM, 7 nM, 8 nM, or 9 nM, or about 10 nM, 20 nM, 30 nM, 40 nM, 50 nM, 60 nM, 70 nM, 80 nM, or 90 nM, as determined by as determined by ELISA.

In some embodiments, the anti-FAM19A5 antibody or antigen binding portion thereof binds to membrane-bound human with a K_{D}, e.g., as determined by ELISA, of 10⁻⁷ M or less, 10⁻⁸ M (10 nM) or less, 10⁻⁹ M (1 nM) or less, 10⁻¹⁰ M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, or 10⁻⁸ M to 10⁻⁷ M. In certain embodiments, the anti-FAM19A5 antibody or antigen binding portion thereof specifically binds to membrane-bound human FAM19A5 with a K_{D} of 10 nM or less as determined by ELISA, e.g., between 0.1 and 10 nM, between 0.1 and 5 nM, between 0.1 and 1 nM, between 0.5 and 10 nM, between 0.5 and 5 nM, between 0.5 and 1 nM, between 1 and 10 nM, between 1 and 5 nM, or between 5 and 10 nM. In some embodiments, the anti-FAM19A5 antibody, or antigen-binding fragment thereof, binds to membrane-bound human FAM19A5 with a K_{D} of about 1 pM, 2 pM, 3 pM, 4 pM, 5 pM, 6 pM, 7 pM, 8 pM, 9 pM, 10 pM, 20 pM, 30 pM, 40 pM, 50 pM, 60 pM, 70 pM, 80 pM, 90 pM, 100 pM, 200 pM, 300 pM, 400 pM, 500 pM, 600 pM, 700 pM, 800 pM, or 900 pM, or about 1 nM, 2 nM, 3 nM, 4 nM, 5 nM, 6 nM, 7 nM, 8 nM, or 9 nM, or about 10 nM, 20 nM, 30 nM, 40 nM, 50 nM, 60 nM, 70 nM, 80 nM, or 90 nM, as determined by ELISA.

In certain embodiments, the anti-FAM19A5 antibody, or antigen-binding fragment thereof, suitable for the methods disclosed herewith cross-competes for binding to (or inhibits binding of) a human FAM19A5 epitope with an anti-FAM19A5 antibody, or antigen-binding fragment thereof, comprising CDRs or variable regions disclosed herein.

In certain embodiments, anti-FAM19A5 antibodies, or antigen-binding fragments thereof, inhibit binding of a reference antibody comprising heavy chain CDR1, CDR2, and CDR3, and light chain CDR1, CDR2, and CDR3, (i) wherein the heavy chain CDR1, CDR2, and CDR3 of the reference antibody comprise the amino acid sequence of SEQ ID NO: 11, SEQ ID NO: 12, and SEQ ID NO: 13, respectively, and light chain CDR1, CDR2, and CDR3 of the reference antibody comprise the amino acid sequence of SEQ ID NO: 23, SEQ ID NO: 24, and SEQ ID NO: 25, respectively; (ii) wherein the heavy chain CDR1 comprises the amino acid sequence of SEQ ID NO: 14, the heavy chain CDR2 comprises the amino acid sequence of SEQ ID NO: 15, and the heavy chain CDR3 comprises the amino acid sequence of SEQ ID NO: 16, the light chain CDR1 comprises the amino acid sequence of SEQ TD NO: 26, the light chain CDR2 comprises the amino acid sequence of SEQ ID NO: 27, and the light chain CDR3 comprises the amino acid sequence of SEQ ID NO: 28; (iii) wherein the heavy chain CDR1 comprises the amino acid sequence of SEQ ID NO: 17, the heavy chain CDR2 comprises the amino acid sequence of SEQ ID NO: 18, and the heavy chain CDR3 comprises the amino acid sequence of SEQ ID NO: 19, the light chain CDR1 comprises the amino acid sequence of SEQ ID NO: 29, the light chain CDR2 comprises the amino acid sequence of SEQ ID NO: 30, and the light chain CDR3 comprises the amino acid sequence of SEQ ID NO: 31 (iv) wherein the heavy chain CDR1 comprises the amino acid sequence of SEQ TD NO 20, the heavy chain CDR2 comprises the amino acid sequence of SEQ ID NO: 21, the heavy chain CDR3 comprises the amino acid sequence of SEQ ID NO: 22, the light chain CDR1 comprises the amino acid sequence of SEQ ID NO: 32, the light chain CDR2 comprises the amino acid sequence of SEQ ID NO: 33, and the light chain CDR3 comprises the amino acid sequence of SEQ TD NO: 34; (v) wherein the heavy chain CDR1 comprises the amino acid sequence of SEQ ID NO: 89, the heavy chain CDR2 comprises the amino acid sequence of SEQ ID NO: 90, the heavy chain CDR3 comprises the amino acid sequence of SEQ TD NO: 91, the light chain CDR1 comprises the amino acid sequence of SEQ ID NO: 92, the light chain CDR2 comprises the amino acid sequence of SEQ ID NO: 93, and the light chain CDR3 comprises the amino acid sequence of SEQ ID NO: 94; (vi) wherein the heavy chain CDR1 comprises the amino acid sequence of SEQ ID NO: 95, the heavy chain CDR2 comprises the amino acid sequence of SEQ TD NO: 96, the heavy chain CDR3 comprises the amino acid sequence of SEQ ID NO: 97, the light chain CDR1 comprises the amino acid sequence of SEQ ID NO: 98, the light chain CDR2 comprises the amino acid sequence of SEQ TD NO: 99, and the light chain CDR3 comprises the amino acid sequence of SEQ ID NO: 100; (vii) wherein the heavy chain CDR1 comprises the amino acid sequence of SEQ ID NO: 101, the heavy chain CDR2 comprises the amino acid sequence of SEQ ID NO: 102, the heavy chain CDR3 comprises the amino acid sequence of SEQ ID NO: 103, the light chain CDR1 comprises the amino acid sequence of SEQ ID NO: 104, the light chain CDR2 comprises the amino acid sequence of SEQ ID NO: 105, and the light chain CDR3 comprises the amino acid sequence of SEQ ID NO: 106; (viii) wherein the heavy chain CDR1 comprises the amino acid sequence of SEQ ID NO: 107, the heavy chain CDR2 comprises the amino acid sequence of SEQ ID NO: 108, the heavy chain CDR3 comprises the amino acid sequence of SEQ ID NO: 109, the light chain CDR1 comprises the amino acid sequence of SEQ TD NO: 110, the light chain CDR2 comprises the amino acid sequence of SEQ ID NO: 111, and the light chain CDR3 comprises the amino acid sequence of SEQ ID NO: 112, (ix) wherein the heavy chain CDR1 comprises the amino acid sequence of SEQ ID NO: 113, the heavy chain CDR2 comprises the amino acid sequence of SEQ ID NO: 114, the heavy chain CDR3 comprises the amino acid sequence of SEQ ID NO: 115, the light chain CDR1 comprises the amino acid sequence of SEQ TD NO: 116, the light chain CDR2 comprises the amino acid sequence of SEQ ID NO: 117, and the light chain CDR3 comprises the amino acid sequence of SEQ ID NO: 118; (x) wherein the heavy chain CDR1 comprises the amino acid sequence of SEQ ID NO: 119, the heavy chain CDR2 comprises the amino acid sequence of SEQ ID NO: 120, the heavy chain CDR3 comprises the amino acid sequence of SEQ ID NO: 121, the light chain CDR1 comprises the amino acid sequence of SEQ ID NO: 122, the light chain CDR2 comprises the amino acid sequence of SEQ ID NO: 123, and the light chain CDR3 comprises the amino acid sequence of SEQ ID NO: 124; (xi) wherein the heavy chain CDR1 comprises the amino acid sequence of SEQ ID NO: 125, the heavy chain CDR2 comprises the amino acid sequence of SEQ ID NO: 126, the heavy chain CDR3 comprises the amino acid sequence of SEQ ID NO: 127, the light chain CDR1 comprises the amino acid sequence of SEQ TD NO: 128, the light chain CDR2 comprises the amino acid sequence of SEQ ID NO: 129, and the light chain CDR3 comprises the amino acid sequence of SEQ ID NO: 130; (xii) wherein the heavy chain CDR1 comprises the amino acid sequence of SEQ ID NO: 131, the heavy chain CDR2 comprises the amino acid sequence of SEQ ID NO: 132, the heavy chain CDR3 comprises the amino acid sequence of SEQ ID NO: 133, the light chain CDR1 comprises the amino acid sequence of SEQ TD NO: 134, the light chain CDR2 comprises the amino acid sequence of SEQ ID NO: 135, and the light chain CDR3 comprises the amino acid sequence of SEQ ID NO: 136; (xiii) wherein the heavy chain CDR1 comprises the amino acid sequence of SEQ ID NO: 137, the heavy chain CDR2 comprises the amino acid sequence of SEQ ID NO: 138, the heavy chain CDR3 comprises the amino acid sequence of SEQ ID NO: 139, the light chain CDR1 comprises the amino acid sequence of SEQ ID NO: 140, the light chain CDR2 comprises the amino acid sequence of SEQ ID NO: 141, and the light chain CDR3 comprises the amino acid sequence of SEQ ID NO: 142; (xiv) wherein the heavy chain CDR1 comprises the amino acid sequence of SEQ ID NO: 143, the heavy chain CDR2 comprises the amino acid sequence of SEQ ID NO: 144, the heavy chain CDR3 comprises the amino acid sequence of SEQ ID NO: 145, the light chain CDR1 comprises the amino acid sequence of SEQ TD NO: 146, the light chain CDR2 comprises the amino acid sequence of SEQ ID NO: 147, and the light chain CDR3 comprises the amino acid sequence of SEQ ID NO: 148; or (xv) wherein the heavy chain CDR1 comprises the amino acid sequence of SEQ ID NO: 149, the heavy chain CDR2 comprises the amino acid sequence of SEQ ID NO: 150, the heavy chain CDR3 comprises the amino acid sequence of SEQ ID NO: 151, the light chain CDR1 comprises the amino acid sequence of SEQ ID NO: 152, the light chain CDR2 comprises the amino acid sequence of SEQ ID NO: 153, and the light chain CDR3 comprises the amino acid sequence of SEQ ID NO: 154.

In some embodiments, the reference antibody comprises (a) heavy and light chain variable region sequences comprising SEQ ID NOs: 35 and 39, respectively; (b) heavy and light chain variable region sequences comprising SEQ ID NOs: 36 and 40, respectively; (c) heavy and light chain variable region sequences comprising SEQ ID NOs: 37 and 41, respectively; (d) heavy and light chain variable region sequences comprising SEQ ID NOs: 38 and 42, respectively; (e) heavy and light chain variable region sequences comprising SEQ ID NOs: 155 and 166, respectively; (f) heavy and light chain variable region sequences comprising SEQ ID NOs: 156 and 167, respectively; (g) heavy and light chain variable region sequences comprising SEQ ID NOs: 157 and 168, respectively; (h) heavy and light chain variable region sequences comprising SEQ ID NOs: 158 and 169, respectively; (i) heavy and light chain variable region sequences comprising SEQ ID NOs: 159 and 170, respectively; (j) heavy and light chain variable region sequences comprising SEQ ID NOs: 160 and 171, respectively; (k) heavy and light chain variable region sequences comprising SEQ ID NOs: 161 and 172, respectively; (1) heavy and light chain variable region sequences comprising SEQ ID NOs: 162 and 173, respectively; (m) heavy and light chain variable region sequences comprising SEQ ID NOs: 163 and 174, respectively; (n) heavy and light chain variable region sequences comprising SEQ ID NOs: 164 and 175, respectively; or (o) heavy and light chain variable region sequences comprising SEQ ID NOs: 165 and 176, respectively.

In some embodiments, the anti-FAM19A5 antibody, or antigen-binding fragment thereof, inhibits binding of such a reference antibody to human FAM19A5 by at least 10%, 20%, 30%, 40%, 50%, 60%, 70%, 80%, or 90% or by 100%. Competing antibodies bind to the same epitope, an overlapping epitope, or to adjacent epitopes (e.g., as evidenced by steric hindrance). Whether two antibodies compete with each other for binding to a target can be determined using competition experiments known in the art such as RIA and EIA.

In certain embodiments, the anti-FAM19A5 antibody, or antigen-binding fragment thereof, binds to the same FAM19A5 epitope as a reference antibody disclosed herein comprising heavy chain CDR1, CDR2, and CDR3 and light chain CDR1, CDR2, and CDR3, (i) wherein the heavy chain CDR1 comprises the amino acid sequence of SEQ ID NO: 11, the heavy chain CDR2 comprises the amino acid sequence of SEQ ID NO: 12, and the heavy chain CDR3 comprises the amino acid sequence of SEQ ID NO: 13, the light chain CDR1 comprises the amino acid sequence of SEQ ID NO: 23, the light chain CDR2 comprises the amino acid sequence of SEQ ID NO: 24, and the light chain CDR3 comprises the amino acid sequence of SEQ ID NO: 25; (ii) wherein the heavy chain CDR1 comprises the amino acid sequence of SEQ ID NO: 14, the heavy chain CDR2 comprises the amino acid sequence of SEQ ID NO: 15, and the heavy chain CDR3 comprises the amino acid sequence of SEQ ID NO: 16, the light chain CDR1 comprises the amino acid sequence of SEQ ID NO: 26, the light chain CDR2 comprises the amino acid sequence of SEQ ID NO: 27, and the light chain CDR3 comprises the amino acid sequence of SEQ ID NO: 28; (iii) wherein the heavy chain CDR1 comprises the amino acid sequence of SEQ ID NO: 17, the heavy chain CDR2 comprises the amino acid sequence of SEQ ID NO: 18, and the heavy chain CDR3 comprises the amino acid sequence of SEQ ID NO: 19, the light chain CDR1 comprises the amino acid sequence of SEQ ID NO: 29, the light chain CDR2 comprises the amino acid sequence of SEQ ID NO: 30, and the light chain CDR3 comprises the amino acid sequence of SEQ ID NO: 31; (iv) wherein the heavy chain CDR1 comprises the amino acid sequence of SEQ ID NO: 20, the heavy chain CDR2 comprises the amino acid sequence of SEQ ID NO: 21, the heavy chain CDR3 comprises the amino acid sequence of SEQ ID NO: 22, the light chain CDR1 comprises the amino acid sequence of SEQ ID NO: 32, the light chain CDR2 comprises the amino acid sequence of SEQ ID NO: 33, and the light chain CDR3 comprises the amino acid sequence of SEQ TD NO: 34; (v) wherein the heavy chain CDR1 comprises the amino acid sequence of SEQ ID NO: 89, the heavy chain CDR2 comprises the amino acid sequence of SEQ ID NO: 90, the heavy chain CDR3 comprises the amino acid sequence of SEQ TD NO: 91, the light chain CDR1 comprises the amino acid sequence of SEQ ID NO: 92, the light chain CDR2 comprises the amino acid sequence of SEQ ID NO: 93, and the light chain CDR3 comprises the amino acid sequence of SEQ ID NO: 94; (vi) wherein the heavy chain CDR1 comprises the amino acid sequence of SEQ ID NO: 95, the heavy chain CDR2 comprises the amino acid sequence of SEQ TD NO: 96, the heavy chain CDR3 comprises the amino acid sequence of SEQ ID NO: 97, the light chain CDR1 comprises the amino acid sequence of SEQ ID NO: 98, the light chain CDR2 comprises the amino acid sequence of SEQ TD NO: 99, and the light chain CDR3 comprises the amino acid sequence of SEQ ID NO: 100; (vii) wherein the heavy chain CDR1 comprises the amino acid sequence of SEQ ID NO: 101, the heavy chain CDR2 comprises the amino acid sequence of SEQ ID NO: 102, the heavy chain CDR3 comprises the amino acid sequence of SEQ ID NO: 103, the light chain CDR1 comprises the amino acid sequence of SEQ ID NO: 104, the light chain CDR2 comprises the amino acid sequence of SEQ ID NO: 105, and the light chain CDR3 comprises the amino acid sequence of SEQ ID NO: 106; (viii) wherein the heavy chain CDR1 comprises the amino acid sequence of SEQ ID NO: 107, the heavy chain CDR2 comprises the amino acid sequence of SEQ ID NO: 108, the heavy chain CDR3 comprises the amino acid sequence of SEQ ID NO: 109, the light chain CDR1 comprises the amino acid sequence of SEQ TD NO: 110, the light chain CDR2 comprises the amino acid sequence of SEQ ID NO: 111, and the light chain CDR3 comprises the amino acid sequence of SEQ ID NO: 112, (ix) wherein the heavy chain CDR1 comprises the amino acid sequence of SEQ ID NO: 113, the heavy chain CDR2 comprises the amino acid sequence of SEQ ID NO: 114, the heavy chain CDR3 comprises the amino acid sequence of SEQ ID NO: 115, the light chain CDR1 comprises the amino acid sequence of SEQ TD NO: 116, the light chain CDR2 comprises the amino acid sequence of SEQ ID NO: 117, and the light chain CDR3 comprises the amino acid sequence of SEQ ID NO: 118; (x) wherein the heavy chain CDR1 comprises the amino acid sequence of SEQ ID NO: 119, the heavy chain CDR2 comprises the amino acid sequence of SEQ ID NO: 120, the heavy chain CDR3 comprises the amino acid sequence of SEQ ID NO: 121, the light chain CDR1 comprises the amino acid sequence of SEQ ID NO: 122, the light chain CDR2 comprises the amino acid sequence of SEQ ID NO: 123, and the light chain CDR3 comprises the amino acid sequence of SEQ ID NO: 124; (xi) wherein the heavy chain CDR1 comprises the amino acid sequence of SEQ ID NO: 125, the heavy chain CDR2 comprises the amino acid sequence of SEQ ID NO: 126, the heavy chain CDR3 comprises the amino acid sequence of SEQ ID NO: 127, the light chain CDR1 comprises the amino acid sequence of SEQ ID NO: 128, the light chain CDR2 comprises the amino acid sequence of SEQ ID NO: 129, and the light chain CDR3 comprises the amino acid sequence of SEQ ID NO: 130; (xii) wherein the heavy chain CDR1 comprises the amino acid sequence of SEQ ID NO: 131, the heavy chain CDR2 comprises the amino acid sequence of SEQ ID NO: 132, the heavy chain CDR3 comprises the amino acid sequence of SEQ ID NO: 133, the light chain CDR1 comprises the amino acid sequence of SEQ ID NO: 134, the light chain CDR2 comprises the amino acid sequence of SEQ ID NO: 135, and the light chain CDR3 comprises the amino acid sequence of SEQ ID NO: 136; (xiii) wherein the heavy chain CDR1 comprises the amino acid sequence of SEQ ID NO: 137, the heavy chain CDR2 comprises the amino acid sequence of SEQ ID NO: 138, the heavy chain CDR3 comprises the amino acid sequence of SEQ ID NO: 139, the light chain CDR1 comprises the amino acid sequence of SEQ ID NO: 140, the light chain CDR2 comprises the amino acid sequence of SEQ ID NO: 141, and the light chain CDR3 comprises the amino acid sequence of SEQ ID NO: 142; (xiv) wherein the heavy chain CDR1 comprises the amino acid sequence of SEQ ID NO: 143, the heavy chain CDR2 comprises the amino acid sequence of SEQ ID NO: 144, the heavy chain CDR3 comprises the amino acid sequence of SEQ ID NO: 145, the light chain CDR1 comprises the amino acid sequence of SEQ ID NO: 146, the light chain CDR2 comprises the amino acid sequence of SEQ ID NO: 147, and the light chain CDR3 comprises the amino acid sequence of SEQ ID NO: 148; or (xv) wherein the heavy chain CDR1 comprises the amino acid sequence of SEQ ID NO: 149, the heavy chain CDR2 comprises the amino acid sequence of SEQ ID NO: 150, the heavy chain CDR3 comprises the amino acid sequence of SEQ ID NO: 151, the light chain CDR1 comprises the amino acid sequence of SEQ ID NO: 152, the light chain CDR2 comprises the amino acid sequence of SEQ ID NO: 153, and the light chain CDR3 comprises the amino acid sequence of SEQ ID NO: 154.

In some embodiments, the reference antibody comprises (a) heavy and light chain variable region sequences comprising SEQ ID NOs: 35 and 39, respectively; (b) heavy and light chain variable region sequences comprising SEQ ID NOs: 36 and 40, respectively; (c) heavy and light chain variable region sequences comprising SEQ ID NOs: 37 and 41, respectively; (d) heavy and light chain variable region sequences comprising SEQ ID NOs: 38 and 42, respectively; (e) heavy and light chain variable region sequences comprising SEQ ID NOs: 155 and 166, respectively; (f) heavy and light chain variable region sequences comprising SEQ ID NOs: 156 and 167, respectively; (g) heavy and light chain variable region sequences comprising SEQ ID NOs: 157 and 168, respectively; (h) heavy and light chain variable region sequences comprising SEQ ID NOs: 158 and 169, respectively; (i) heavy and light chain variable region sequences comprising SEQ ID NOs: 159 and 170, respectively; (j) heavy and light chain variable region sequences comprising SEQ ID NOs: 160 and 171, respectively; (k) heavy and light chain variable region sequences comprising SEQ ID NOs: 161 and 172, respectively; (1) heavy and light chain variable region sequences comprising SEQ ID NOs: 162 and 173, respectively; (m) heavy and light chain variable region sequences comprising SEQ ID NOs: 163 and 174, respectively; (n) heavy and light chain variable region sequences comprising SEQ ID NOs: 164 and 175, respectively; or (o) heavy and light chain variable region sequences comprising SEQ ID NOs: 165 and 176, respectively.

Techniques for determining whether two antibodies bind to the same epitope include, e.g., epitope mapping methods, such as, x-ray analyses of crystals of antigen:antibody complexes which provides atomic resolution of the epitope and hydrogen/deuterium exchange mass spectrometry (HDX-MS), methods monitoring the binding of the antibody to antigen fragments or mutated variations of the antigen, where loss of binding due to a modification of an amino acid residue within the antigen sequence is often considered an indication of an epitope component, computational combinatorial methods for epitope mapping.

An anti-FAM19A5 antibody, or antigen-binding fragment thereof, that would be useful in the methods disclosed herewith can bind to at least one epitope of mature human FAM19A5, as determined, e.g., by binding of the antibodies to fragments of human FAM19A5. In some embodiments, anti-FAM19A5 antibodies, or antigen-binding fragments thereof, bind to a fragment located within the amino acid sequence of TLDRDSSQPRRTIARQTARC (SEQ ID NO: 6 or amino acid residues 42 to 61 of SEQ ID NO: 2), e.g., an epitope having at least 1, 2, 3, 4, 5, 6, 7, 8, 9, 10, 11, 12, 13, 14, 15, 16, 17, 18, 19, or 20 amino acids of SEQ ID NO: 6. In some embodiments, anti-FAM19A5 antibodies or antigen binding portion thereof bind to SEQ ID NO: 6 at one or more amino acids corresponding to amino acid residues 46 to 51 (i.e., DSSQPR), e.g., amino acid residues 46, 50, and 52 (i.e., D---P-R), e.g., amino acid residues 46, 47, 48, and 50 (i.e., DSS-P) of SEQ ID NO: 2. In some embodiments, anti-FAM19A5 antibodies, or antigen-binding fragments thereof, bind to a fragment located within the amino acid sequence of CDMLPCLEGEGCDLLINRSG (SEQ ID NO: 9 or amino acids 90 to 109 of SEQ ID NO: 2), e.g., an epitope having at least 1, 2, 3, 4, 5, 6, 7, 8, 9, 10, 11, 12, 13, 14, 15, 16, 17, 18, 19, or 20 amino acids of SEQ ID NO: 9. In certain embodiments, anti-FAM19A5 antibodies or antigen binding portion thereof bind to SEQ ID NO: 9 at one or more amino acids residues 99 to 107 (i.e., EGCDLLINR), e.g., amino acid residues 102, 103, 105, and 107 (i.e., DL-I-R), e.g., amino acid residues 99, 100, 102, 103, 105, and 107 (i.e., EG-DL-I-R), e.g., amino acid residues 99, 100, and 107 (i.e., EG------R) of SEQ ID NO: 4.

In some embodiments, the at least one epitope has the amino acid sequence that is at least 90%, at least about 95%, at least about 96%, at least about 97%, at least about 98%, at least about 99%, or about 100% identical to SEQ ID NO: 6. In some embodiments, the at least one epitope has the amino acid sequence that is at least 90%, at least about 95%, at least about 96%, at least about 97%, at least about 98%, at least about 99%, or about 100% identical to SEQ ID NO: 9.

In some embodiments, the anti-FAM19A5 antibody, or antigen-binding fragment thereof, binds to a human FAM19A5 epitope only, which is SEQ ID NO: 5, 6, 7, 8, 9, or 10, or a fragment located within the amino acid sequence of SEQ ID NO: 5, 6, 7, 8, 9, or 10, e.g., an epitope having 1, 2, 3, 4, 5, 6, 7, 8, 9, 10, 11, 12, 13, 14, 15, 16, 17, 18, 19, or 20 amino acids of SEQ ID NO: 5, 6, 7, 8, 9, or 10.

In some embodiments, the anti-FAM19A5 antibody, or antigen-binding fragment thereof, of the present disclosure binds to SEQ ID NO: 6 or a fragment thereof in its native conformation (i.e., un-denatured). In some embodiments, the anti-FAM19A5 antibody, or antigen-binding fragment thereof, of the present disclosure binds to SEQ ID NO: 9 or a fragment thereof in its native conformation (i.e., un-denatured). In some embodiments, the anti-FAM19A5 antibody, or antigen-binding fragment thereof, binds to both glycosylated and unglycosylated human FAM19A5.

In some embodiments, the anti-FAM19A5 antibody, or antigen-binding fragment thereof, further binds to one or more additional FAM19A5 epitopes. Therefore, certain anti-FAM19A5 antibodies, or antigen-binding fragments thereof, bind to an epitope of SEQ ID NO: 6 and an additional epitope or an epitope of SEQ ID NO: 9 and an additional epitope. Other anti-FAM19A5 antibodies, or antigen-binding fragments thereof, can bind to an epitope of SEQ ID NO: 5, SEQ ID NO: 9, and an additional epitope. In some embodiments, anti-FAM19A5 antibodies, or antigen-binding fragments thereof, bind to an epitope of SEQ ID NO: 6, an epitope of SEQ ID NO: 10, and an additional epitope. In some embodiments, the one or more additional FAM19A5 epitopes are selected from QLAAGTCEIVTLDR (SEQ ID NO: 5, epitope F1), TLDRDSSQPRRTIARQTARC (SEQ ID NO: 6, epitope F2), TARCACRKGQIAGTTRARPA (SEQ ID NO: 7, epitope F3), ARPACVDARIIKTKQWCDML (SEQ ID NO: 8, epitope F4), CDMLPCLEGEGCDLLINRSG (SEQ ID NO: 9, epitope F5), or NRSGWTCTQPGGRIKTTTVS (SEQ ID NO: 10, epitope F6), or a fragment located within the amino acid sequence of SEQ ID NO: 5, SEQ ID NO: 6, SEQ ID NO: 7, SEQ ID NO: 8, SEQ ID NO: 9, SEQ ID NO: 10, or any combination thereof. A fragment located within the amino acid sequence of SEQ ID NO: 5, SEQ ID NO: 6, SEQ ID NO: 7, SEQ ID NO: 8, SEQ ID NO: 9, or SEQ ID NO: 10, includes a fragment having 1, 2, 3, 4, 5, 6, 7, 8, 9, 10, 11, 12, 13, 14, 15, 16, 17, 18, 19, or 20 amino acids of any of SEQ ID NO: 5, SEQ ID NO: 6, and SEQ ID NO: 7, SEQ ID NO: 8, SEQ ID NO: 9, or SEQ ID NO: 10. In some embodiments, the one or more additional FAM19A5 epitopes are selected from SEQ ID NO: 5, 6, 7, 8, 9, or 10, or a fragment located within the amino acid sequence of SEQ ID NO: 5, 6, 7, 8, 9, or 10, e.g., a fragment having 1, 2, 3, 4, 5, 6, 7, 8, 9, 10, 11, 12, 13, 14, 15, 16, 17, 18, 19, or 20 amino acids of SEQ ID NO: 5, 6, 7, 8, 9, or 10, or any combination thereof. In some embodiments, the anti-FAM19A5 antibody, or antigen-binding fragment thereof, of the present disclosure binds to any of the one or more additional epitopes in their native conformation (i.e., un-denatured). In some embodiments, the anti-FAM19A5 antibody, or antigen-binding fragments thereof, binds to both glycosylated and unglycosylated of the one or more additional FAM19A5 epitopes.

In some embodiments, anti-FAM19A5 antibodies or antigen binding portions thereof bind to at least one FAM19A5 epitope identified as EP2, EP4, and/or EP8, wherein EP2 comprises, consists essentially of, or consists of the amino acids DSSQP (SEQ ID NO: 66), wherein EP4 comprises, consists essentially of, or consists of the amino acids ARCACRK (SEQ ID NO: 68), and wherein EP8 comprises, consists essentially of, or consists of the amino acids TCTQPGGR (SEQ ID NO: 72). In some embodiments, the at least one epitope has the amino acid sequence that is at least 90%, at least about 95%, at least about 96%, at least about 97%, at least about 98%, at least about 99%, or about 100% identical to EP2, EP4, or EP8. In some embodiments, anti-FAM19A5 antibodies or antigen binding portion thereof only bind to EP2. In some embodiments, anti-FAM19A5 antibodies or antigen binding portion thereof bind to EP4 and EP8.

In some embodiments, the anti-FAM19A5 antibody, or antigen binding portion thereof, binds to at least one FAM19A5 epitope identified as EP6, EP7, or EP8, wherein EP6 comprises the amino acids KTKQWCDML (SEQ ID NO: 70), wherein EP7 comprises the amino acids GCDLLINR (SEQ ID NO: 71), and wherein EP8 comprises the amino acids TCTQPGGR (SEQ ID NO: 72). In some embodiments, the at least one epitope has the amino acid sequence that is at least 90%, at least about 95%, at least about 96%, at least about 97%, at least about 98%, at least about 99%, or about 100% identical to EP6, EP7, or EP8. In some embodiments, the anti-FAM19A5 antibody, or antigen binding portion thereof, only binds to EP6, EP7, or EP8. In some embodiments, the anti-FAM19A5 antibody, or antigen binding portion thereof, binds to EP6, EP7, and EP8. In some embodiments, the anti-FAM19A5 antibody, or antigen binding portion thereof, binds to EP7 and EP8. In some embodiments, the anti-FAM19A5 antibody, or antigen binding portion thereof, binds to EP7.

In some embodiments, anti-FAM19A5 antibodies or antigen binding portion thereof bind to one or more FAM19A5 epitopes selected from the group consisting of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and any combinations thereof.

In some embodiments, provided herein is an antibody, or antigen-binding fragment thereof, that binds to FAM19A5 (e.g., human FAM19A5) with a 20%, 25%, 30%, 35%, 40%, 45%, 50%, 55%, 60%, 65%, 70%, 75%, 80%, 85%, 90%, 95% or higher affinity than to another protein in the FAM19A family as measured by, e.g., a immunoassay (e.g., ELISA), surface plasmon resonance, or kinetic exclusion assay. In a certain embodiments, the anti-FAM19A5 antibody, or antigen-binding fragment thereof, binds to FAM19A5 (e.g., human FAM19A5) with no cross reactivity with another protein in the FAM19A family as measured by, e.g., an immunoassay.

In some embodiments, the anti-FAM19A5 antibodies are not native antibodies or are not naturally-occurring antibodies. For example, the anti-FAM19A5 antibodies have post-translational modifications that are different from those of antibodies that are naturally-occurring, such as by having more, less or a different type of post-translational modification.

### V. Exemplary Anti-FAM19A5 Antibodies

Particular antibodies that can be used in the methods disclosed herein are antibodies, e.g., monoclonal antibodies, having the CDR and/or variable region sequences disclosed herein, as well as antibodies having at least 80% identity (e.g., at least 85%, at least 90%, at least 95%, or at least 99% identity) to their variable region or CDR sequences. The VH and VL amino acid sequences of different anti-FAM19A5 antibodies of the present disclosure are provided in Tables 4 and 5, respectively.

TABLE 2
Variable heavy chain CDR amino acid sequences

| Antibody | VH-CDR1 | VH-CDR2 | VH-CDR3 |
|---|---|---|---|
| Anti- | SHGMF | EITNDGSGT | STYECPGGFS |
| FAM19A5 | (SEQ ID | NYGSAVKG | CWGDTGQIDA |
| (″2-13″) | NO: 11) | (SEQ ID | (SEQ ID |
| | | NO: 12) | NO: 13) |
| Anti- | SFNMF | QISSSGSST | SSYDCPYGHCS |
| FAM19A5 | (SEQ ID | NYAPAVRG | SGVDSAGEIDA |
| (″3-2″) | NO: 14) | (SEQ ID | (SEQ ID |
| | | NO: 15) | NO: 16) |
| Anti- | SYQMG | VINKS | GSASYIT |
| FAM19A5 | (SEQ ID | GSDTS | AATIDA |
| (″1-65″) | NO: 17) | (SEQ ID | (SEQ ID |
| | | NO: 18) | NO: 19) |
| Anti- | GFDFSDYG | IRSD | AKDGNGYCALD |
| FAM19A5 | (SEQ ID | GSNP | AYRSGGYSCGV |
| (″1-28″) | NO: 20) | (SEQ ID | (SEQ ID |
| | | NO: 21) | NO: 22) |
| Anti- | TYAVT | YINWRGGT | DASSGAAF |
| FAM19A5 | (SEQ ID | SYANWAKG | GSYGMDP |
| (″P2-C12″) | NO: 89) | (SEQ ID | (SEQ ID |
| | | NO: 90) | NO: 91) |
| Anti- | SSNWWS | EIYHGGTT | WQLVGGLDV |
| FAM19A5 | (SEQ ID | NYNPSLKG | (SEQ ID |
| (″13B4″) | NO: 95) | (SEQ ID | NO: 97) |
| | | NO: 96) | |
| Anti- | GYSWT | EISHFGSA | ALRGTYSR |
| FAM19A5 | (SEQ ID | NYNPSLKS | FYYGMDV |
| (″13F7″) | NO: 101) | (SEQ ID | (SEQ ID |
| | | NO: 102) | NO: 103) |
| Anti- | SYYWS | YIYPSGST | VNPFGY |
| FAM19A5 | (SEQ ID | NYNPSLKS | YYAMDV |
| (″15A9″) | NO: 107) | (SEQ ID | (SEQ ID |
| | | NO: 108) | NO: 109) |
| Anti- | SDYMS | IIYPSTTT | GSNWS |
| FAM19A5 | (SEQ ID | YYASWAKG | SGMNL |
| (″P1-A03″) | NO: 113) | (SEQ ID | (SEQ ID |
| | | NO: 114) | NO: 115) |
| Anti- | TYYMS | IVYPSGTT | GDSF |
| FAM19A5 | (SEQ ID | YYANWAKG | GYGL |
| (″P1-A08″) | NO: 119) | (SEQ ID | (SEQ ID |
| | | NO: 120) | NO: 121) |
| Anti- | NYYMG | IIYASGST | IDIGVGDYG |
| FAM19A5 | (SEQ ID | YYASWAKG | WAYDRLDL |
| (″P1-F02″) | NO: 125) | (SEQ ID | (SEQ ID |
| | | NO: 126) | NO: 127) |
| Anti- | GYYMS | IIYPSGST | VAGYVGYG |
| FAM19A5 | (SEQ ID | DYASWAKG | YETFFDI |
| (″P2-A01″) | NO: 131) | (SEQ ID | (SEQ ID |
| | | NO: 132) | NO: 133) |
| Anti- | NYDMS | FMDTDGSA | RGSSYY |
| FAM19A5 | (SEQ ID | YYATWAKG | GGIDI |
| (″P2-A03″) | NO: 137) | (SEQ ID | (SEQ ID |
| | | NO: 138) | NO: 139) |
| Anti- | SYYMN | IIYPSGTT | TVSGYFDI |
| FAM19A5 | (SEQ ID | YYAGWAKG | (SEQ ID |
| (″P2-F07″) | NO: 143) | (SEQ ID | NO: 145) |
| | | NO: 144) | |
| Anti- | SYGVS | YIANNYNP | DNYGMDP |
| FAM19A5 | (SEQ ID | HYASWAKG | (SEQ ID |
| (″P2-F11″) | NO: 149) | (SEQ ID | NO: 151) |
| | | NO: 150) | |

TABLE 3
Variable light chain CDR amino acid sequences

| Antibody | VL-CDR1 | VL-CDR2 | VL-CDR3 |
|---|---|---|---|
| Anti- | SGGSYSYG | WDDERPS | GTEDIS |
| FAM19A5 | (SEQ ID | (SEQ ID | GTAGV |
| (″2-13″) | NO: 23) | NO: 24) | (SEQ ID |
| | | | NO: 25) |
| Anti- | SGGGSYA | ESNKRPS | GSWDS |
| FAM19A5 | GSYYYG | (SEQ ID | SNGGI |
| (″3-2″) | (SEQ ID | NO: 27) | (SEQ ID |
| | NO: 26) | | NO: 28) |
| Anti- | SGGGSS | WNDKRPS | GNDDYSS |
| FAM19A5 | GYGYG | (SEQ ID | DSGYVGV |
| (″1-65″) | (SEQ ID | NO: 30) | (SEQ ID |
| | NO: 29) | | NO: 31) |
| Anti- | GYGYG | QND | GSEDSS |
| FAM19A5 | (SEQ ID | (SEQ ID | TLAGI |
| (″1-28″) | NO: 32) | NO: 33) | (SEQ ID |
| | | | NO: 34) |
| Anti- | QASQSI | EASKLAS | QQGYSS |
| FAM19A5 | SSYLS | (SEQ ID | TNVWNA |
| (″P2-C12″) | (SEQ ID | NO: 93) | (SEQ ID |
| | NO: 92) | | NO: 94) |
| Anti- | SGDKLG | QDNKRPS | QAWDS |
| FAM19A5 | NVYAS | (SEQ ID | STAV |
| (″13B4″) | (SEQ ID | NO: 99) | (SEQ ID |
| | NO: 98) | | NO: 100) |
| Anti- | RSSQSLLH | LGSNRAS | MQARQ |
| FAM19A5 | SNGYNYLD | (SEQ ID | TPLT |
| (″13F7″) | (SEQ ID | NO: 105) | (SEQ ID |
| | NO: 104) | | NO: 106) |
| Anti- | RASQSI | GASTLQS | QESAS |
| FAM19A5 | STSLN | (SEQ ID | IPRT |
| (″15A9″) | (SEQ ID | NO: 111) | (SEQ ID |
| | NO: 110) | | NO: 112) |
| Anti- | LASEDI | GASNLES | LGGYSY |
| FAM19A5 | YSGIS | (SEQ ID | SSTGLT |
| (″P1-A03″) | (SEQ ID | NO: 117) | (SEQ ID |
| | NO: 116) | | NO: 118) |
| Anti- | TADTLS | RDTSRPS | ATSDGSG |
| FAM19A5 | RSYAS | (SEQ ID | SNYQYV |
| (″P1-A08″) | (SEQ ID | NO: 123) | (SEQ ID |
| | NO: 122) | | NO: 124) |
| Anti- | LASEDI | GASNLES | LGGYS |
| FAM19A5 | YSGIS | (SEQ ID | YSSIT |
| (″P1-F02″) | (SEQ ID | NO: 129) | (SEQ ID |
| | NO: 128 | | NO: 130) |
| Anti- | LASEDI | GASNLES | LGGVTYS |
| FAM19A5 | YSGIS | (SEQ ID | STGTHLT |
| (″P2-A01″) | (SEQ ID | NO: 135) | (SEQ ID |
| | NO: 134) | | NO: 136) |
| Anti- | QASQSI | RASTLAS | QSPAYDP |
| FAM19A5 | GGNLA | (SEQ ID | AAYVGNA |
| (″P2-A03″) | (SEQ ID | NO: 141) | (SEQ ID |
| | NO: 140) | | NO: 142) |
| Anti- | LASEDI | GTSNLES | QGYSS |
| FAM19A5 | YSALA | (SEQ ID | YPLT |
| (″P2-F07″) | (SEQ ID | NO: 147) | (SEQ ID |
| | NO: 146) | | NO: 148) |
| Anti- | QASQSVY | AASTLAS | QGEFSCS |
| FAM19A5 | NNKNLA | (SEQ ID | SADCNA |
| (″P2-F11″) | (SEQ ID | NO: 153) | (SEQ ID |
| | NO: 152) | | NO: 154) |

TABLE 4
Variable heavy chain amino acid sequence

| Antibody | VH Amino Acid Sequence (SEQ ID NO) |
|---|---|
| Anti- | AVTLDESGGGLQTPGGALSLVCKASGFTFSSHGMFWVR |
| FAM19A5 | QTPGKGLEYVAEITNDGSGTNYGSAVKGRATISRDNGQ |
| (″2-13″) | STVRLQLNNLRAEDTGTYFCARSTYECPGGFSCWGDTG |
| | QIDAWGHGTEVIVSS (SEQ ID NO: 35) |
| Anti- | AVTLDESGGGLQTPGGALSLVCKASGFTFSSFNMFWVR |
| FAM19A5 | QAPGKGLEYVAQISSSGSSTNYAPAVRGRATISRDNGQ |
| (″3-2″) | STVRLQLNNPGAEDTGTYYCAKSSYDCPYGHCSSGVDS |
| | AGEIDAWGHGTEVIVSS (SEQ ID NO: 36) |
| Anti- | AVTLDESGGGLQTPGGALSLVCKASGFTFSSYQMGWVR |
| FAM19A5 | QAPGKGLEWVGVINKSGSDTSYGSAVKGRATISRDNGQ |
| (″1-65″) | STVRLQLNNLRAEDTGTYFCAKGSASYITAATIDAWGH |
| | GTEVIVSS (SEQ ID NO: 37) |
| Anti- | AVTLDESGGGLQTPGGALSLVCKASGFDFSDYGMGWVR |
| FAM19A5 | QAPGKGLEWVAAIRSDGSNPSYGSAVKGRATISKDNGR |
| (″1-28″) | STVRLQLNNLRAEDTATYYCAKDGNGYCALDAYRSGGY |
| | SCGVYPGSIDAWGHGTEVIVSS (SEQ ID NO: 38) |
| Anti- | QSLEESGGRLVTPGTPLTLTCTVSGFSLSTYAVTWVRQ |
| FAM19A5 | APGKGLEWIGYINWRGGTSYANWAKGRFTISKTSSTTV |
| (″P2- | DLKMTSPTTEDTATYFCARDASSGAAFGSYGMDPWGPG |
| C12″) | TLVTVSS (SEQ ID NO: 155) |
| Anti- | QVQLQESGPGLVKPSGTLSLNCAVSGGSISSSNWWSWV |
| FAM19A5 | RQPPGKGLEWIGEIYHGGTTNYNPSLKGRVTMSVDKTK |
| (″13B4″) | NQFSLRLSSVTAVDTAVYYCARWQLVGGLDVWGQGTTV |
| | TVSS (SEQ ID NO: 156) |
| Anti- | QVQLQEWGAGLLKPSETLSLTCAINAESFNGYSWTWIR |
| FAM19A5 | QTPGKGLEWIGEISHFGSANYNPSLKSRATISADKSKN |
| (″13F7″) | QFSLKLTSVTAVDTAVYYCARALRGTYSRFYYGMDVWG |
| | QGTTVTVSS (SEQ ID NO: 157) |
| Anti- | QVQLQESGPGLVKPSETLSLTCTVSGGSISSYYWSWIR |
| FAM19A5 | QPPGKGLEWIGYIYPSGSTNYNPSLKSRVTISVDTSKN |
| (″15A9″) | QFSLNLKSVTAVDTAVYYCARVNPFGYYYAMDVWGQGT |
| | TVTVSS (SEQ ID NO: 158) |
| Anti- | QSVEESGGRLVTPGTPLTLTCTVSGFSLSSDYMSWVRQ |
| FAM19A5 | APGEGLEWIGIIYPSTTTYYASWAKGRFTISKTSSTTV |
| (″P1- | ELKMTSLTTEDTATYFCARGSNWSSGMNLWGPGTLVTV |
| A03″) | SS (SEQ ID NO: 159) |
| Anti- | QSLEESGGRLVTPGTPLTLTCTASGFSLSTYYMSWVRQ |
| FAM19A5 | APGKGLEWIGIVYPSGTTYYANWAKGRFTISTASTTVD |
| (″P1- | LMITSPTTEDTATYFCARGDSFGYGLWGPGTLVTVSS |
| A08″) | (SEQ ID NO: 160) |
| Anti- | QSLEESGGRLVTPGTPLTLTCTASGFSLSNYYMGWVRQ |
| FAM19A5 | APGEGLEWIGIIYASGSTYYASWAKGRFTISKTSTTVD |
| (″P1- | LKMTSLTTEDTATYFCARIDIGVGDYGWAYDRLDLWGQ |
| F02″) | GTLVTVSS (SEQ ID NO: 161) |
| Anti- | QEQLVESGGRLVTPGTPLTLSCTASGFFLSGYYMSWVR |
| FAM19A5 | QAPGKGLEWIGIIYPSGSTDYASWAKGRFTISKTSTTV |
| (″P2- | DLKITTPTTEDTATYFCARVAGYVGYGYETFFDIWGPG |
| A01″) | TLVTVSL (SEQ ID NO: 162) |
| Anti- | QSVEESGGRLVTPGTPLTLTCTVSGFSLNNYDMSWVRQ |
| FAM19A5 | APGKGLEYIGFMDTDGSAYYATWAKGRFTISRTSTTVD |
| (″P2- | LKMTSPTTEDTATYFCARRGSSYYGGIDIWGPGTPVTV |
| A03″) | SL (SEQ ID NO: 163) |
| Anti- | QSLEESGGRLVTPGTPLTLTCTASGFSLSSYYMNWVRQ |
| FAM19A5 | APGKGLEWIGIIYPSGTTYYAGWAKGRFTISKTSTTVD |
| (″P2- | LKITSPTSEDTATYFCARTVSGYFDIWGPGTLVTVSL |
| F07″) | (SEQ ID NO: 164) |
| Anti- | QEQLVESGGRLVTPGTTLTLTCTVSGFSLSSYGVSWVR |
| FAM19A5 | QAPGKGLEWIGYIANNYNPHYASWAKGRFTISKTSSTT |
| (″P2- | VDLKMTSLTTEDTATYFCARDNYGMDPWGPGTLVTVSS |
| F11″) | (SEQ ID NO: 165) |

TABLE 5
Variable light chain amino acid sequence

| Antibody | VL Amino Acid Sequence (SEQ ID NO) |
|---|---|
| Anti- | ALTQPSSVSANPGETVKITCSGGSYSYGWFQQKSPGSA |
| FAM19A5 | LVTVIYWDDERPSDIPSRFSGALSGSTNTLTITGVQAD |
| (″2-13″) | DEAVYFCGTEDISGTAGVFGAGTTLTVL |
| | (SEQ ID NO: 39) |
| Anti- | ALTQPSSVSANPGETVKITCSGGGSYAGSYYYGWYQQK |
| FAM19A5 | APGSAPVTLIYESNKRPSDIPSRFSGSTSGSTATLTIT |
| (″3-2″) | GVQADDEAIYYCGSWDSSNGGIFGAGTTLTVL |
| | (SEQ ID NO: 40) |
| Anti- | ALTQPSSVSANPGETVKITCSGGGSSGYGYGWYQQKSP |
| FAM19A5 | SSAPLTVIYWNDKRPSDIPSRFSGSKSGSTHTLTITGV |
| (″1-65″) | QAEDEAVYFCGNDDYSSDSGYVGVFGAGTTLTVL |
| | (SEQ ID NO: 41) |
| Anti- | ALTQPSSVSANLEGTVEITCSGSGYGYGWYQQKSPGSA |
| FAM19A5 | PVTVIYQNDKRPSDIPSRFSGSKSGSTGTLTITGVQVE |
| (″1-28″) | DEAVYYCGSEDSSTLAGIFGAGTTLTVL |
| | (SEQ ID NO: 42) |
| Anti- | ELDMTQTPSSVSAAVGGTVTIKCQASQSISSYLSWYQQ |
| FAM19A5 | KPGQPPKLLIYEASKLASGVPSRFSGSGYGTEFLTISD |
| (″P2- | LECADAATYYCQQGYSSTNVWNAFGGGTNVEIK |
| C12″) | (SEQ ID NO: 166) |
| Anti- | SYELTQPLSVSVSPGQTASITCSGDKLGNVYASWYQQK |
| FAM19A5 | PGQSPTLVIYQDNKRPSGIPERFSGSNSGKTATLTISG |
| (″13B4″) | TQALDEADYYCQAWDSSTAVFGGGTKLTVL |
| | (SEQ ID NO: 167) |
| Anti- | DIVMTQTPLSLPVAPGEPASISCRSSQSLLHSNGYNYL |
| FAM19A5 | DWYVQKPGQPPQLLIYLGSNRASGVPDRFSGSGSGTDF |
| (″13F7″) | TLKISRVEAEDVGVYYCMQARQTPLTFGGGTKVEIK |
| | (SEQ ID NO: 168) |
| Anti- | DIQMTQSPSSLSASVGDRITISCRASQSISTSLNWYQQ |
| FAM19A5 | TPGKAPRLLIYGASTLQSGVPSRFSGGGSGTDFSLTIT |
| (″15A9″) | SLQPEDFATYYCQESASIPRTFGQGTKLDIK |
| | (SEQ ID NO: 169) |
| Anti- | ELVMTQTPPSLSASVGETVRIRCLASEDIYSGISWYQQ |
| FAM19A5 | KPEKPPTLLISGASNLESGVPPRFSGSGSGTDYTLTIG |
| (″P1- | GVQAEDAATYYCLGGYSYSSTGLTFGAGTNVEIK |
| A03″) | (SEQ ID NO: 170) |
| Anti- | ELVLTQSPSVQVNLGQTVSLTCTADTLSRSYASWYQQK |
| FAM19A5 | PGQAPVLLIYRDTSRPSGVPDRFSGSSSGNTATLTISG |
| (″P1- | AQAGDEADYYCATSDGSGSNYQYVFGGGTQLTVT |
| A08″) | (SEQ ID NO: 171) |
| Anti- | ELDMTQTPPSLSASVGETVRIRCLASEDIYSGISWYQQ |
| FAM19A5 | KPGKPPTLLIYGASNLESGVPPRFSGSGSGTDYTLTIG |
| (″P1- | GVQAEDAATYYCLGGYSYSSITFGAGTNVEIK |
| F02″) | (SEQ ID NO: 172) |
| Anti- | ELVMTQTPPSLSASVGETVRIRCLASEDIYSGISWYQQ |
| FAM19A5 | KPGKPPTLLIYGASNLESGVPPRFSGSGSGSDYTLTIG |
| (″P2- | GVQAEDAATYYCLGGVTYSSTGTHLTFGAGTNVEIK |
| A01″) | (SEQ ID NO: 173) |
| Anti- | ELDLTQTPASVSEPVGGTVTIKCQASQSIGGNLAWYQQ |
| FAM19A5 | KPGQPPKLLIYRASTLASGVPSRFKGSGSGTDFTLTIS |
| (″P2- | DLECADAATYYCQSPAYDPAAYVGNAFGGGTELEIL |
| A03″) | (SEQ ID NO: 174) |
| Anti- | ELDLTQTPPSLSASVGGTVTINCLASEDIYSALAWYQQ |
| FAM19A5 | KPGKPPTLLISGTSNLESGVPPRFSGSGSGTDYTLTIG |
| (″P2- | GVQAEDAATYFCQGYSSYPLTFGAGTNVEIK |
| F07″) | (SEQ ID NO: 175) |
| Anti- | ELDLTQTPSSVSAAVGGTVTINCQASQSVYNNKNLAWY |
| FAM19A5 | QQKPGQPPKLLIYAASTLASGVSSRFKGSGSGTQFTLT |
| (″P2- | ISDVQCDDAATYYCQGEFSCSSADCNAFGGGTELEIL |
| F11″) | (SEQ ID NO: 176) |

In some embodiments, the anti-FAM19A5 antibody, or antigen-binding fragment thereof, comprises heavy and light chain variable regions, wherein the heavy chain variable region comprises the amino acid sequence of SEQ ID NOs: 35-38 or 155-165. In other embodiments, the isolated anti-FAM19A5 antibody, or an antigen binding portion thereof, comprises the CDRs of the heavy chain variable region selected from the group consisting of SEQ ID NOs: 35-38 or 155-165.

In some embodiments, the anti-FAM19A5 antibody, or antigen-binding fragment thereof, comprises heavy and light chain variable regions, wherein the light chain variable region comprises the amino acid sequence of SEQ ID NOs: 39-42 or 166-176. In other embodiments, the isolated anti-FAM19A5 antibody, or an antigen binding portion thereof, comprises the CDRs of the light chain variable region selected from the group consisting of SEQ ID NOs: 39-42 or 166-176.

In certain embodiments, the isolated anti-FAM19A5 antibody, or an antigen binding portion thereof, comprises the CDRs of the heavy chain variable region selected from the group consisting of SEQ ID NOs: 35-38 or 155-165 and the CDRs of the light chain variable region selected from the group consisting of SEQ ID NOs: 39-42 or 166-176.

In some embodiments, the anti-FAM19A5 antibody, or antigen-binding fragment thereof, comprises heavy and light chain variable regions, (i) wherein the heavy chain variable region comprises the amino acid sequence of SEQ ID NO: 35 and wherein the light chain variable region comprises the amino acid sequence of SEQ ID NO: 39; (ii) wherein the heavy chain variable region comprises the amino acid sequence of SEQ ID NO: 36 and wherein the light chain variable region comprises the amino acid sequence of SEQ ID NO: 40; (iii) wherein the heavy chain variable region comprises the amino acid sequence of SEQ ID NO: 37 and wherein the light chain variable region comprises the amino acid sequence of SEQ ID NO: 41; (iv) wherein the heavy chain variable region comprises the amino acid sequence of SEQ ID NO: 38 and wherein the light chain variable region comprises the amino acid sequence of SEQ ID NO: 42; (v) wherein the heavy chain variable region comprises the amino acid sequence of SEQ ID NO: 155 and wherein the light chain variable region comprises the amino acid sequence of SEQ ID NO: 166; (vi) wherein the heavy chain variable region comprises the amino acid sequence of SEQ ID NO: 156 and wherein the light chain variable region comprises the amino acid sequence of SEQ ID NO: 167; (vii) wherein the heavy chain variable region comprises the amino acid sequence of SEQ ID NO: 157 and wherein the light chain variable region comprises the amino acid sequence of SEQ ID NO: 168; (viii) wherein the heavy chain variable region comprises the amino acid sequence of SEQ ID NO: 158 and wherein the light chain variable region comprises the amino acid sequence of SEQ ID NO: 169; (ix) wherein the heavy chain variable region comprises the amino acid sequence of SEQ ID NO: 159 and wherein the light chain variable region comprises the amino acid sequence of SEQ ID NO: 170; (x) wherein the heavy chain variable region comprises the amino acid sequence of SEQ ID NO: 160 and wherein the light chain variable region comprises the amino acid sequence of SEQ ID NO: 171; (xi) wherein the heavy chain variable region comprises the amino acid sequence of SEQ ID NO: 161 and wherein the light chain variable region comprises the amino acid sequence of SEQ ID NO: 172; (xii) wherein the heavy chain variable region comprises the amino acid sequence of SEQ ID NO: 162 and wherein the light chain variable region comprises the amino acid sequence of SEQ ID NO: 173; (xiii) wherein the heavy chain variable region comprises the amino acid sequence of SEQ ID NO: 163 and wherein the light chain variable region comprises the amino acid sequence of SEQ ID NO: 174; (xiv) wherein the heavy chain variable region comprises the amino acid sequence of SEQ ID NO: 164 and wherein the light chain variable region comprises the amino acid sequence of SEQ ID NO: 175; and (xv) wherein the heavy chain variable region comprises the amino acid sequence of SEQ ID NO: 165 and wherein the light chain variable region comprises the amino acid sequence of SEQ ID NO: 176.

In some embodiments, the anti-FAM19A5 antibody, or antigen-binding fragment thereof, comprises a heavy chain variable region and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NOs: 35-38 or 155-165.

In some embodiments, the anti-FAM19A5 antibody, or antigen-binding fragment thereof, comprises a heavy chain variable region and a light chain variable region, wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NOs: 39-42 or 166-176.

In some embodiments, the anti-FAM19A5 antibody, or antigen-binding fragment thereof, comprises heavy and light chain variable regions, wherein the heavy chain variable region comprises an amino acid sequence which is at least about 80%, at least about 85%, at least about 90%, at least about 95%, at least about 96%, at least about 97% at least about 98%, at least about 99%, or about 100% identical to the amino acid sequence set forth as SEQ ID NOs: 35-38 or 155-165, and wherein the light chain variable region comprises an amino acid sequence which is at least about 80%, at least about 85%, at least about 90%, at least about 95%, at least about 96%, at least about 97% at least about 98%, at least about 99%, or about 100% identical to the amino acid sequence set forth as SEQ ID NOs: 39-42 or 166-176.

In some embodiments, the anti-FAM19A5 antibody, or an antigen-binding fragment thereof, comprises:
(a) heavy and light chain variable region sequences comprising SEQ ID NOs: 35 and 39, respectively;
(b) heavy and light chain variable region sequences comprising SEQ ID NOs: 36 and 40, respectively;
(c) heavy and light chain variable region sequences comprising SEQ ID NOs: 37 and 41, respectively;
(d) heavy and light chain variable region sequences comprising SEQ ID NOs: 38 and 42, respectively;
(e) heavy and light chain variable region sequences comprising SEQ ID NOs: 155 and 166, respectively;
(f) heavy and light chain variable region sequences comprising SEQ ID NOs: 156 and 167, respectively;
(g) heavy and light chain variable region sequences comprising SEQ ID NOs: 157 and 168, respectively;
(h) heavy and light chain variable region sequences comprising SEQ ID NOs: 158 and 169, respectively;
(i) heavy and light chain variable region sequences comprising SEQ ID NOs: 159 and 170, respectively;
(j) heavy and light chain variable region sequences comprising SEQ ID NOs: 160 and 171, respectively;
(k) heavy and light chain variable region sequences comprising SEQ ID NOs: 161 and 172, respectively;
(l) heavy and light chain variable region sequences comprising SEQ ID NOs: 162 and 173, respectively;
(m) heavy and light chain variable region sequences comprising SEQ ID NOs: 163 and 174, respectively;
(n) heavy and light chain variable region sequences comprising SEQ ID NOs: 164 and 175, respectively; or
(o) heavy and light chain variable region sequences comprising SEQ ID NOs: 165 and 176, respectively.

In some embodiments, the anti-FAM19A5 antibody, or antigen-binding fragment thereof, of the present disclosure comprises (i) the heavy chain CDR1, CDR2 and CDR3 of 2-13, or combinations thereof, and/or the light chain CDR1, CDR2, and CDR3 of 2-13, or any combinations thereof, (ii) the heavy chain CDR1, CDR2 and CDR3 of 3-2, or combinations thereof, and/or the light chain CDR1, CDR2, and CDR3 of 3-2, or any combinations thereof, (iii) the heavy chain CDR1, CDR2 and CDR3 of 1-65, or combinations thereof, and/or the light chain CDR1, CDR2, and CDR3 of 1-65, or any combinations thereof, (iv) the heavy chain CDR1, CDR2 and CDR3 of 1-28, or combinations thereof, and/or the light chain CDR1, CDR2, and CDR3 of 1-28, or any combinations thereof, (v) the heavy chain CDR1, CDR2, and CDR3 of P2-C12, or combinations thereof, and/or the light chain CDR1, CDR2, and CDR3 of P2-C12, or any combinations thereof, (vi) the heavy chain CDR1, CDR2, and CDR3 of 13B4, or combinations thereof, and/or the light chain CDR1, CDR2, and CDR3 of 13B4, or any combinations thereof, (vii) the heavy chain CDR1, CDR2, and CDR3 of 13F7, or combinations thereof, and/or the light chain CDR1, CDR2, and CDR3 of 13F7, or any combinations thereof, (viii) the heavy chain CDR1, CDR2, and CDR3 of 15A9, or combinations thereof, and/or the light chain CDR1, CDR2, and CDR3 of 15A9, or any combinations thereof, (ix) the heavy chain CDR1, CDR2, and CDR3 of P1-A03, or combinations thereof, and/or the light chain CDR1, CDR2, and CDR3 of P1-A03, or any combinations thereof, (x) the heavy chain CDR1, CDR2, and CDR3 of P1-A08, or combinations thereof, and/or the light chain CDR1, CDR2, and CDR3 of P1-A08, or any combinations thereof, (xi) the heavy chain CDR1, CDR2, and CDR3 of P1-F02, or combinations thereof, and/or the light chain CDR1, CDR2, and CDR3 of P1-F02, or any combinations thereof, (xii) the heavy chain CDR1, CDR2, and CDR3 of P2-A01, or combinations thereof, and/or the light chain CDR1, CDR2, and CDR3 of P2-A01, or any combinations thereof, (xiii) the heavy chain CDR1, CDR2, and CDR3 of P2-A03, or combinations thereof, and/or the light chain CDR1, CDR2, and CDR3 of P2-A03, or any combinations thereof, (xiv) the heavy chain CDR1, CDR2, and CDR3 of P2-F07, or combinations thereof, and/or the light chain CDR1, CDR2, and CDR3 of P2-F07, or any combinations thereof, or (xv) the heavy chain CDR1, CDR2, and CDR3 of P2-F11, or combinations thereof, and/or the light chain CDR1, CDR2, and CDR3 of F2-F11, or any combinations thereof. The amino acid sequences of the VH CDR1, CDR2, and CDR3 for the different anti-FAM19A5 antibodies disclosed herein are provided in Table 2. The amino acid sequences of the VL CDR1, CDR2, and CDR3 for the different anti-FAM19A5 antibodies disclosed herein are provided in Table 3.

In some embodiments, the anti-FAM19A5 antibody, or antigen-binding fragment thereof, which specifically binds to human FAM19A5, comprises:
(a) a VH CDR1 comprising the amino acid sequence of SEQ ID NO: 11;
(b) a VH CDR2 comprising the amino acid sequence of SEQ ID NO: 12; and/or
(c) a VH CDR3 comprising the amino acid sequence of SEQ ID NO: 13.

In some embodiments, the antibody, or antigen-binding fragment thereof, comprises one, two, or all three of the VH CDRs above.

In some embodiments, the anti-FAM19A5 antibody, or antigen-binding fragment thereof, which specifically binds to human FAM19A5, comprises:
(a) a VL CDR1 comprising the amino acid sequence of SEQ ID NO: 23;
(b) a VL CDR2 comprising the amino acid sequence of SEQ ID NO: 24; and/or
(c) a VL CDR3 comprising the amino acid sequence of SEQ ID NO: 25.

In some embodiments, the antibody, or antigen-binding fragment thereof, comprises one, two, or all three of the VL CDRs above.

In some embodiments, the anti-FAM19A5 antibody, or antigen-binding fragment thereof, which specifically binds to human FAM19A5, comprise:
(a) a VH CDR1 comprising the amino acid sequence of SEQ ID NO: 11;
(b) a VH CDR2 comprising the amino acid sequence of SEQ ID NO: 12;
(c) a VH CDR3 comprising the amino acid sequence of SEQ ID NO: 13;
(d) a VL CDR1 comprising the amino acid sequence of SEQ ID NO: 23;
(e) a VL CDR2 comprising the amino acid sequence of SEQ ID NO: 24; and/or
(f) a VL CDR3 comprising the amino acid sequence of SEQ ID NO: 25.

In some embodiments, the anti-FAM19A5 antibody, or antigen-binding fragment thereof, of the disclosure, which specifically binds to human FAM19A5, comprises:
(a) a VH CDR1 comprising the amino acid sequence of SEQ ID NO: 14;
(b) a VH CDR2 comprising the amino acid sequence of SEQ ID NO: 15; and/or
(c) a VH CDR3 comprising the amino acid sequence of SEQ ID NO: 16.

In some embodiments, the antibody, or antigen-binding fragment thereof, comprises one, two, or all three of the VH CDRs above.

In some embodiments, the anti-FAM19A5 antibody, or antigen-binding fragment thereof, which specifically binds to human FAM19A5, comprises:
(a) a VL CDR1 comprising the amino acid sequence of SEQ ID NO: 26;
(b) a VL CDR2 comprising the amino acid sequence of SEQ ID NO: 27; and/or
(c) a VL CDR3 comprising the amino acid sequence of SEQ ID NO: 28.

In some embodiments, the antibody, or antigen-binding fragment thereof, comprises one, two, or all three of the VL CDRs above.

In some embodiments, the anti-FAM19A5 antibody, or antigen-binding fragment thereof, which specifically binds to human FAM19A5, comprise:
(a) a VH CDR1 comprising the amino acid sequence of SEQ ID NO: 14;
(b) a VH CDR2 comprising the amino acid sequence of SEQ ID NO: 15;
(c) a VH CDR3 comprising the amino acid sequence of SEQ ID NO: 16;
(d) a VL CDR1 comprising the amino acid sequence of SEQ ID NO: 26;
(e) a VL CDR2 comprising the amino acid sequence of SEQ ID NO: 27; and/or
(f) a VL CDR3 comprising the amino acid sequence of SEQ ID NO: 28.

In some embodiments, the anti-FAM19A5 antibody, or antigen-binding fragment thereof, of the disclosure, which specifically binds to human FAM19A5, comprises:
(a) a VH CDR1 comprising the amino acid sequence of SEQ ID NO: 17;
(b) a VH CDR2 comprising the amino acid sequence of SEQ ID NO: 18; and/or
(c) a VH CDR3 comprising the amino acid sequence of SEQ ID NO: 19.

In some embodiments, the antibody, or antigen-binding fragment thereof, comprises one, two, or all three of the VH CDRs above.

In some embodiments, the anti-FAM19A5 antibody, or antigen-binding fragment thereof, which specifically binds to human FAM19A5, comprises:
(a) a VL CDR1 comprising the amino acid sequence of SEQ ID NO: 29;
(b) a VL CDR2 comprising the amino acid sequence of SEQ ID NO: 30; and/or
(c) a VL CDR3 comprising the amino acid sequence of SEQ ID NO: 31.

In some embodiments, the antibody, or antigen-binding fragment thereof, comprises one, two, or all three of the VL CDRs above.

In some embodiments, the anti-FAM19A5 antibody. or antigen-binding fragment thereof, which specifically binds to human FAM19A5, comprise:
(a) a VH CDR1 comprising the amino acid sequence of SEQ ID NO: 17;
(b) a VH CDR2 comprising the amino acid sequence of SEQ ID NO: 18;
(c) a VH CDR3 comprising the amino acid sequence of SEQ ID NO: 19;
(d) a VL CDR1 comprising the amino acid sequence of SEQ ID NO: 29;
(e) a VL CDR2 comprising the amino acid sequence of SEQ ID NO: 30; and/or
(f) a VL CDR3 comprising the amino acid sequence of SEQ ID NO: 31.

In some embodiments, the anti-FAM19A5 antibody, or antigen-binding fragment thereof, of the disclosure, which specifically binds to human FAM19A5, comprises:
(a) a VH CDR1 comprising the amino acid sequence of SEQ ID NO: 20;
(b) a VH CDR2 comprising the amino acid sequence of SEQ ID NO: 21; and/or
(c) a VH CDR3 comprising the amino acid sequence of SEQ ID NO: 22.

In some embodiments, the antibody, or antigen-binding fragment thereof, comprises one, two, or all three of the VH CDRs above.

In some embodiments, the anti-FAM19A5 antibody, or antigen-binding fragment thereof, which specifically binds to human FAM19A5, comprises:
(a) a VL CDR1 comprising the amino acid sequence of SEQ ID NO: 32;
(b) a VL CDR2 comprising the amino acid sequence of SEQ ID NO: 33; and/or
(c) a VL CDR3 comprising the amino acid sequence of SEQ ID NO: 34.

In some embodiments, the antibody, or antigen-binding fragment thereof, comprises one, two, or all three of the VL CDRs above.

In some embodiments, the anti-FAM19A5 antibody, or antigen-binding fragment thereof, which specifically binds to human FAM19A5, comprise:
(a) a VH CDR1 comprising the amino acid sequence of SEQ ID NO: 20;
(b) a VH CDR2 comprising the amino acid sequence of SEQ ID NO: 21;
(c) a VH CDR3 comprising the amino acid sequence of SEQ ID NO: 22;
(d) a VL CDR1 comprising the amino acid sequence of SEQ ID NO: 32;
(e) a VL CDR2 comprising the amino acid sequence of SEQ ID NO: 33; and/or
(f) a VL CDR3 comprising the amino acid sequence of SEQ ID NO: 34.

In some embodiments, the anti-FAM19A5 antibody, or antigen-binding fragment thereof, comprises one, two, three, four, five, or six of the CDRs above.

A VH domain, or one or more CDRs thereof, described herein can be linked to a constant domain for forming a heavy chain, e.g., a full length heavy chain. Similarly, a VL domain, or one or more CDRs thereof, described herein can be linked to a constant domain for forming a light chain, e.g., a full length light chain. A full length heavy chain and full length light chain combine to form a full length antibody.

Accordingly, in some embodiments, the anti-FAM19A5 antibody comprises an antibody light chain and heavy chain, e.g., a separate light chain and heavy chain. With respect to the light chain, in certain embodiments, the light chain of an antibody described herein is a kappa light chain. In some embodiments, the light chain of an antibody described herein is a lambda light chain. In some embodiments, the light chain of an antibody described herein is a human kappa light chain or a human lambda light chain. In certain embodiments, an antibody described herein, which specifically binds to an FAM19A5 polypeptide (e.g., human FAM19A5) comprises a light chain which comprises any VL or VL CDR amino acid sequences described herein, and wherein the constant region of the light chain comprises the amino acid sequence of a human kappa light chain constant region. In some embodiments, an antibody described herein, which specifically binds to an FAM19A5 polypeptide (e.g., human FAM19A5) comprises a light chain which comprises a VL or VL CDR amino acid sequences described herein, and wherein the constant region of the light chain comprises the amino acid sequence of a human lambda light chain constant region. Non-limiting examples of human constant region sequences have been described in the art, e.g., see U.S. Pat. No. 5,693,780 and Kabat E A et al, (1991) supra.

With respect to the heavy chain, in some embodiments, the heavy chain of an antibody described herein can be an alpha (α), delta (δ), epsilon (ε), gamma (γ) or mu (μ) heavy chain. In another specific embodiment, the heavy chain of an antibody described can comprise a human alpha (α), delta (δ), epsilon (ε), gamma (γ) or mu (μ) heavy chain. In one embodiment, an antibody described herein, which specifically binds to FAM19A5 (e.g., human FAM19A5), comprises a heavy chain which comprises a VH or VH CDR amino acid sequence described herein, and wherein the constant region of the heavy chain comprises the amino acid sequence of a human gamma (γ) heavy chain constant region. In some embodiments, an antibody described herein, which specifically binds to FAM19A5 (e.g., human FAM19A5), comprises a heavy chain which comprises a VH or VH CDR amino acid sequence disclosed herein, and wherein the constant region of the heavy chain comprises the amino acid of a human heavy chain described herein or known in the art. Non-limiting examples of human constant region sequences have been described in the art, see e.g., U.S. Pat. No. 5,693,780 and Kabat E A et al., (1991) supra.

In some embodiments, the antibody described herein, which specifically binds to FAM19A5 (e.g., human FAM19A5), comprises a VL domain and a VH domain comprising the VH or VH CDRs and VL and VL CDRs described herein, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In certain embodiments, an antibody described herein, which specifically binds to FAM19A5 (e.g., human FAM19A5) comprises a VL domain and a VH domain comprising any amino acid sequences described herein, and wherein the constant regions comprise the amino acid sequences of the constant regions of an IgG, IgE, IgM, IgD, IgA or IgY immunoglobulin molecule, any subclass (e.g., IgG1, IgG2, IgG3, IgG4, IgA1 and IgA2) of immunoglobulin molecule. In some embodiments, the constant regions comprise the amino acid sequences of the constant regions of a human IgG, which are naturally-occurring, including subclasses (e.g., IgG1, IgG2, IgG3 or IgG4), and allotypes (e.g., G1m, G2m, G3m, and nG4m) and variants thereof. See e.g., Vidarsson G. et al. Front Immunol. 5:520 (published online Oct. 20, 2014) and Jefferis R. and Lefranc M P, mAbs 1:4, 1-7(2009). In some embodiments, the constant regions comprise the amino acid sequences of the constant regions of a human IgG1, IgG2, IgG3, or IgG4, or variants thereof.

In certain embodiments, the anti-FAM19A5 antibody, or antigen-binding fragment thereof, disclosed herein does not have Fc effector functions, e.g., complement-dependent cytotoxicity (CDC) and/or antibody-dependent cellular phagocytosis (ADCP). Effector functions are mediated by the Fc region and the residues most proximal to the hinge region in the CH2 domain of the Fc region are responsible for effector functions of antibodies as it contains a largely overlapping binding site for C1q (complement) and IgG-Fc receptors (FcγR) on effector cells of the innate immune system. Also, IgG2 and IgG4 antibodies have lower levels of Fc effector functions than IgG1 and IgG3 antibodies. Effector functions of an antibody can be reduced or avoided by different approaches known in the art, including (1) using antibody fragments lacking the Fc region (e.g., such as a Fab, F(ab′)₂, single chain Fv (scFv), or a sdAb consisting of a monomeric VH or VL domain); (2) generating aglycosylated antibodies, which can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells, see e.g., U.S. Pub. No. 20120100140); (3) employing Fc regions from an IgG subclass that have reduced effector function (e.g., an Fc region from IgG2 or IgG4 antibodies or a chimeric Fc region comprising a CH2 domain from IgG2 or IgG4 antibodies, see e.g., U.S. Pub. No. 20120100140 and Lau C. et al. J. Immunol. 191:4769-4777 (2013)); and (4) generating an Fc region with mutations that result in reduced or no Fc functions. See e.g., U.S. Pub. No. 20120100140 and U.S. and PCT applications cited therein and An et al., mAbs 1:6, 572-579 (2009).

Thus, in some embodiments, the antigen-binding fragment thereof disclosed herein is an Fab, an Fab′, an F(ab′)₂, an Fv, a single chain Fv (scFv), or a sdAb consisting of a monomeric VH or VL domain. Such antibody fragments are well known in the art and are described supra.

In some embodiments, the anti-FAM19A5 antibody, or antigen-binding fragment thereof, disclosed herein comprises an Fc region with reduced or no Fc effector function. In some embodiments, the constant regions comprise the amino acid sequences of the Fc region of a human IgG2 or IgG4, in some embodiments, the anti-FAM19A5 antibody is of an IgG2/IgG4 isotype. In some embodiments, the anti-FAM19A5 antibody comprises a chimeric Fc region which comprises a CH2 domain from an IgG antibody of the IgG4 isotype and a CH3 domain from an IgG antibody of the IgG1 isotype, or a chimeric Fc region which comprises a hinge region from IgG2 and a CH2 region from IgG4, or an Fc region with mutations that result in reduced or no Fc functions. Fc regions with reduced or no Fc effector function include those known in the art. See e.g., Lau C. et al. J. Immunol. 191:4769-4777 (2013); An et al., mAbs 1:6, 572-579 (2009); and U.S. Pub. No. 20120100140 and the U.S. patents and publications and PCT publications cited therein. Also Fc regions with reduced or no Fc effector function can be readily made by a person of ordinary skill in the art.

### VI. Nucleic Acid Molecules

Another aspect described herein pertains to one or more nucleic acid molecules that encode any one of the antibodies, or antigen-binding fragments thereof, described herein. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See F. Ausubel et al., ed. Current Protocols in Molecular Biology, Greene Publishing and Wiley Interscience, New York (1987). A nucleic acid described herein can be, for example, DNA or RNA and can or cannot contain intronic sequences. In certain embodiments, the nucleic acid is a cDNA molecule.

Nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Certain nucleic acids molecules described herein are those encoding the VH and VL sequences of the various anti-FAM19A5 antibodies of the present disclosure. Exemplary DNA sequences encoding the VH sequence of such antibodies are set forth in SEQ ID NOs: 43-46 and 177. Exemplary DNA sequences encoding the VL sequences of such antibodies are set forth in SEQ ID NOs: 47-50 and 178.

TABLE 6
Variable heavy chain polynucleotide sequence

| Anti- | Variable Heavy Chain Polynucleotide |
|---|---|
| body | Sequence (SEQ ID NO) |
| Anti- | GCCGTGACGTTGGACGAGTCCGGGGGCGGCCTCCAGACG |
| FAM19A5 | CCCGGAGGAGCGCTCAGCCTCGTCTGCAAGGCCTCCGGG |
| (2-13) | TTCACCTTCAGCAGCCATGGCATGTTCTGGGTGCGACAG |
| | ACGCCCGGCAAGGGGTTGGAATATGTCGCTGAAATTACC |
| | AATGATGGTAGTGGCACAAACTACGGGTCGGCGGTGAAG |
| | GGCCGTGCCACCATCTCGAGGGACAACGGGCAGAGCACA |
| | GTGAGGCTGCAGCTGAACAACCTCAGGGCTGAGGACACC |
| | GGCACCTACTTCTGCGCCAGATCTACTTATGAATGTCCT |
| | GGTGGTTTTAGTTGTTGGGGTGATACTGGTCAAATAGAC |
| | GCATGGGGCCACGGGACCGAAGTCATCGTCTCCTCCA |
| | (SEQ ID NO: 43) |
| Anti- | GCCGTGACGTTGGACGAGTCCGGGGGCGGCCTCCAGACG |
| FAM19A5 | CCCGGAGGAGCGCTCAGCCTCGTCTGCAAGGCCTCCGGG |
| (3-2) | TTCACCTTCAGCAGCTTCAACATGTTCTGGGTGCGACAG |
| | GCGCCCGGCAAGGGGCTGGAATACGTCGCTCAAATTAGC |
| | AGCAGTGGTAGTAGCACAAACTACGCACCCGCGGTGAGG |
| | GGCCGTGCCACCATCTCGAGGGACAACGGGCAGAGCACA |
| | GTGAGGCTGCAGCTGAACAACCCCGGGGCTGAAGACACC |
| | GGCACCTACTACTGCGCCAAAAGTAGTTATGACTGTCCT |
| | TACGGTCATTGTAGTAGTGGTGTTGATAGTGCTGGTGAG |
| | ATCGACGCATGGGGCCACGGGACCGAAGTCATCGTCTCC |
| | TCCA (SEQ ID NO: 44) |
| Anti- | GCCGTGACACTGGACGAATCTGGGGGAGGGCTGCAGACT |
| FAM19A5 | CCAGGCGGAGCTCTGAGCCTGGTGTGCAAGGCATCCGGG |
| (1-65) | TTCACCTTTAGCTCCTACCAGATGGGATGGGTGCGGCAG |
| | GCACCAGGGAAGGGCCTGGAGTGGGTCGGAGTGATCAAC |
| | AAATCTGGGAGTGACACAAGCTACGGCAGCGCCGTGAAG |
| | GGAAGGGCCACCATCAGCAGGGACAATGGCCAGAGTACC |
| | GTGCGGCTGCAGCTGAACAATCTGCGCGCTGAGGACACT |
| | GGCACCTACTTCTGTGCTAAGGGATCAGCAAGCTATATC |
| | ACAGCCGCTACTATTGATGCATGGGGACACGGGACAGAA |
| | GTCATCGTGTCTAGT (SEQ ID NO: 45) |
| Anti- | GCCGTGACGTTGGACGAGTCCGGGGGCGGCCTCCAGACG |
| FAM19A5 | CCCGGAGGAGCGCTCAGCCTCGTCTGCAAGGCCTCCGGG |
| (1-28) | TTCGACTTCAGCGATTATGGCATGGGTTGGGTGCGACAG |
| | GCTCCAGGCAAGGGGCTGGAGTGGGTTGCTGCTATTAGA |
| | AGTGATGGTAGTAACCCATCATACGGGTCGGCGGTGAAG |
| | GGCCGTGCCACCATCTCGAAGGACAACGGGCGAAGCACA |
| | GTGAGGCTGCAGCTGAACAACCTCAGGGCTGAGGACACC |
| | GCCACCTACTACTGCGCCAAGGATGGTAATGGTTACTGT |
| | GCTCTCGATGCTTATCGTAGTGGTGGTTATAGTTGTGGT |
| | GTTTATCCTGGTAGCATCGACGCATGGGGCCACGGGACC |
| | GAAGTCATCGTCTCCTCC (SEQ ID NO: 46) |
| Anti- | CAGTCGCTGGAGGAGTCCGGGGGTCGCCTGGTCACGCCT |
| FAM19A5 | GGGACACCCCTGACACTCACCTGCACCGTCTCTGGATTC |
| (P2- | TCCCTCAGTACCTATGCAGTGACCTGGGTCCGCCAGGCT |
| C12) | CCAGGGAAGGGGCTGGAATGGATCGGATACATTAATTGG |
| | CGTGGTGGGACATCCTACGCGAACTGGGCGAAAGGCCGA |
| | TTCACCATCTCCAAAACCTCGTCGACCACGGTGGATCTG |
| | AAAATGACCAGTCCGACAACCGAGGACACGGCCACCTAT |
| | TTCTGTGCCAGAGATGCTAGTAGTGGTGCTGCTTTTGGG |
| | TCTTACGGCATGGACCCCTGGGGCCCAGGGACCCTCGTC |
| | ACCGTCTCTTCA (SEQ ID NO: 177) |

TABLE 7
Variable light chain polynucleotide sequence

| Anti- | Variable Light Chain Polynucleotide |
|---|---|
| body | Sequence (SEQ ID NO) |
| Anti- | GGCCCTGACTCAGCCGTCCTCGGTGTCAGCAAACCCAGG |
| FAM19A5 | AGAAACCGTCAAGATAACCTGCTCCGGGGGTAGCTATAG |
| (2-13) | CTATGGCTGGTTCCAGCAGAAGTCTCCTGGCAGTGCCCT |
| | TGTCACTGTGATCTACTGGGATGATGAGAGACCCTCGGA |
| | CATCCCTTCACGATTCTCCGGTGCCCTATCCGGCTCCAC |
| | AAACACATTAACCATCACTGGGGTCCAAGCCGACGACGA |
| | GGCTGTCTATTTCTGTGGGACTGAAGACATCAGCGGCAC |
| | TGCTGGTGTATTTGGGGCCGGGACAACCCTGACCGTCCT |
| | GGG (SEQ ID NO: 47) |
| Anti- | GGCCCTGACTCAGCCGTCCTCGGTGTCAGCAAACCCAGG |
| FAM19A5 | AGAAACCGTCAAGATCACCTGCTCCGGGGGTGGCAGCTA |
| (3-2) | TGCTGGAAGTTACTATTATGGCTGGTACCAGCAGAAGGC |
| | ACCTGGCAGTGCCCCTGTCACTCTGATCTATGAAAGCAA |
| | CAAGAGACCCTCGGACATCCCTTCACGATTCTCCGGTTC |
| | CACATCTGGCTCCACAGCCACACTAACCATCACTGGGGT |
| | CCAAGCCGATGACGAGGCTATCTATTACTGTGGGAGCTG |
| | GGACAGTAGCAATGGTGGTATATTTGGGGCCGGGACAAC |
| | CCTGACCGTCCTAGG (SEQ ID NO: 48) |
| Anti- | GCCCTGACTCAGCCCTCTTCCGTGTCAGCCAACCCTGGA |
| FAM19A5 | GAAACTGTGAAGATCACCTGCAGCGGAGGAGGGAGCTCC |
| (1-65) | GGATACGGATATGGGTGGTATCAGCAGAAATCCCCATCT |
| | AGTGCCCCCCTGACTGTGATCTATTGGAACGACAAGAGG |
| | CCTAGTGATATTCCATCAAGATTCAGTGGATCAAAAAGC |
| | GGGTCCACTCACACCCTGACAATCACTGGCGTGCAGGCA |
| | GAGGACGAAGCCGTCTACTTCTGCGGAAATGACGATTAC |
| | TCAAGCGATTCTGGCTATGTGGGCGTCTTTGGCGCAGGA |
| | ACCACACTGACAGTGCTG (SEQ ID NO: 49) |
| Anti- | GCCCTGACTCAGCCGTCCTCGGTGTCAGCAAACCTGGAA |
| FAM19A5 | GGAACCGTCGAGATCACCTGCTCCGGGAGTGGCTATGGT |
| (1-28) | TATGGCTGGTATCAGCAGAAGTCTCCTGGCAGTGCCCCT |
| | GTCACTGTGATCTATCAGAACGACAAGAGACCCTCGGAC |
| | ATCCCTTCACGATTCTCCGGTTCCAAATCCGGCTCCACG |
| | GGCACATTAACCATCACTGGGGTCCAAGTCGAGGACGAG |
| | GCTGTCTATTACTGTGGGAGTGAAGACAGCAGCACTCTT |
| | GCTGGTATATTTGGGGCCGGGACAACCCTGACCGTCCTA |
| | (SEQ ID NO: 50) |
| Anti- | GAGCTCGATATGACCCAGACTCCATCCTCCGTGTCTGCA |
| FAM19A5 | GCTGTGGGAGGCACAGTCACCATCAAGTGCCAGGCCAGT |
| (P2- | CAGAGCATTAGTAGCTACTTATCCTGGTATCAGCAGAAA |
| C12) | CCAGGGCAGCCTCCCAAGCTCCTGATCTATGAAGCATCC |
| | AAACTGGCCTCTGGGGTCCCATCGCGGTTCAGCGGCAGT |
| | GGATATGGGACAGAGTTCACTCTCACCATCAGCGACCTG |
| | GAGTGTGCCGATGCTGCCACTTACTACTGTCAACAGGGT |
| | TATAGTAGTACTAATGTTTGGAATGCTTTCGGCGGAGGC |
| | ACCAATGTGGAAATCAAA |
| | (SEQ ID NO: 178) |

A method for making an anti-FAM19A5 antibody, or antigen-binding fragment thereof, as disclosed herewith can comprise expressing the relevant heavy chain and light chain of the antibody in a cell line comprising the nucleotide sequences encoding the heavy and light chains with a signal peptide, e.g., SEQ ID NOs: 43 and 47, SEQ ID NOs: 44 and 48, SEQ ID NOs: 45 and 49, SEQ ID NOs: 46 and 50, SEQ ID NOs: 177 and 178, respectively. Host cells comprising these nucleotide sequences are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, for example, an IgG2 and/or IgG 4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly4-Ser)₃, such that the VH and VL sequences can be expressed as a contiguous single-chain protein, with the VL and VH regions joined by the flexible linker (see, e.g., Bird et al., Science 242:423-426 (1988); Huston et al., Proc. Natl. Acad. Sci. USA 85:5879-5883 (1988); McCafferty et al., Nature 348:552-554 (1990)).

In some embodiments, the vector disclosed herein comprises an isolated nucleic acid molecule comprising a nucleotide sequence encoding an antibody, or antigen-binding fragment thereof. In certain embodiments, the vectors can be used for gene therapy.

Suitable vectors for the disclosure include expression vectors, viral vectors, and plasmid vectors. In one embodiment, the vector is a viral vector.

As used herein, an “expression vector” refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

### VII. Antibody Production

Anti-FAM19A5 antibodies, or antigen-binding fragments thereof, disclosed herein can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some embodiments, an antibody described herein is an antibody (e.g., recombinant antibody) prepared, expressed, created, or isolated by any means that involves creation, e.g., via synthesis, genetic engineering of DNA sequences. In certain embodiments, such antibody comprises sequences (e.g., DNA sequences or amino acid sequences) that do not naturally exist within the antibody germline repertoire of an animal or mammal (e.g., human) in vivo.

### VIII. Pharmaceutical Compositions

Provided herein are compositions comprising an antibody or antigen-binding fragment thereof described herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In a specific embodiment, pharmaceutical compositions comprise an antibody or antigen-binding fragment thereof, a bispecific molecule, or a immunoconjugate described herein, and optionally one or more additional prophylactic or therapeutic agents, in a pharmaceutically acceptable carrier. In a specific embodiment, pharmaceutical compositions comprise an effective amount of an antibody or antigen-binding fragment thereof described herein, and optionally one or more additional prophylactic of therapeutic agents, in a pharmaceutically acceptable carrier. In some embodiments, the antibody is the only active ingredient included in the pharmaceutical composition. Pharmaceutical compositions described herein can be useful in reducing a FAM19A5 activity and thereby treat a mood disorder, such as major depressive disorder or bipolar disorder.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition can be formulated for any route of administration to a subject. Specific examples of routes of administration include intranasal, oral, parenterally, intrathecally, intra-cerebroventricularly, pulmonarily, subcutaneously, or intraventricularly. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of an antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An antibody or antigen-binding fragment thereof described herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one embodiment, have diameters of less than 50 microns, in one embodiment less than 10 microns.

An antibody, or antigen-binding fragment thereof, described herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

In certain embodiments, a pharmaceutical composition comprising an antibody or antigen-binding fragment thereof described herein is a lyophilized powder, which can be reconstituted for administration as solutions, emulsions and other mixtures. It can also be reconstituted and formulated as solids or gels. The lyophilized powder is prepared by dissolving an antibody or antigen-binding fragment thereof described herein, or a pharmaceutically acceptable derivative thereof, in a suitable solvent. In some embodiments, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The antibodies, or antigen-binding fragments thereof, the bispecific molecule, or the immunoconjugate described herein and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In a specific embodiment, the anti-FAM19A5 antibody or antigen-binding fragment thereof described herein can be used treat a mood disorder, such as major depressive disorder or bipolar disorder.

The compositions to be used for in vivo administration can be sterile. This is readily accomplished by filtration through, e.g., sterile filtration membranes.

### IX. Kits

Provided herein are kits comprising one or more antibodies described herein, or antigen-binding fragments thereof, wherein the kits are for diagnostic or treatment. In a specific embodiment, provided herein is a pack or kit comprising one or more containers filled with one or more of the ingredients of the compositions described herein, such as one or more antibodies provided herein or an antigen-binding fragment thereof, optional an instructing for use. In some embodiments, the kits contain a composition described herein and any diagnostic, prophylactic or therapeutic agent, such as those described herein.

### EXAMPLES

The following experimental methods and details are referenced in the Examples that follow.

### Example 1

### Expression and Purification of Human FAM19A5 Protein

Recombinant human FAM19A5 protein was produced and purified and the purified protein was used in an antibody screening assay based on binding affinity analysis. First, LPS-hT plasmid containing the FAM19A5 gene was transformed into bacteria and protein over-expression was induced. Once produced, the FAM19A5 protein was purified using an Ni-NTA affinity chromatography (Qiagen, Valencia, CA, USA). Using gradually higher concentration of imidazole, we removed the His-tagged FAM19A5 protein from the Ni-column. The protein expression in the solution is measured using Coomassie Brilliant Blue R-250 Dye. Taking only the FAM19A5 immidazole containing solution, we concentrated the FAM19A5 protein using PBS. When the concentration was complete, both the purity and concentration of the FAM19A5 protein were measured using a Western Blot assay. The concentrated protein was subsequently used to screen for FAM19A5-specific antibodies.

### Example 2

### Production of Antibody Libraries FAM19A5

A FAM19A5 peptide was synthesized, conjugated at the C-terminal end to KLH (Anygen Co., Ltd.), and used as antigen for immunization of a chicken. 50 μg of the synthetic peptide KLH conjugate was mixed in 750 μl phosphate buffered saline (PBS) and incubated at 37° C. for 30 minutes. Afterwards, the toxin is removed in a 2% squalene endotoxin MPL (monophosphorylate lipid A species) and mycobacteria (mycobacteria) of the cell wall components of TDW and CWS containing a water-in-oil emulsion adjuvant (RIBI+MPL+TDM+CWS adjuvant, Sigma, St. Louis, Mo, USA) in emulsified, which was then subcutaneously injected into three chickens. The chickens were immunized for a total of three times, approximately 2-3 weeks apart between immunization. The titer of the antibodies obtained from the immunized chickens was measured via immune blotting using lysates of HEK293T cells which overexpressed the FAM19A5 protein. Sera from chickens that received the three immunizations were used as primary antibody. The secondary antibody used was anti-chicken IgG(Y) polyclonal antibody conjugated to HRP (Horseradish peroxidase) (Rabbit anti-chicken IgG (Y)-HRP, Millipore corporation, Billeria, MA, USA).

Single-chain variable fragment (scFv) library was prepared from immunized chicken Using TRI reagent (Invitrogen, Carlsbad, CA USA), we extracted RNAs from the spleen, bone marrow, and synovial sac of the immunized chickens described above. Oligo-dT primers and SUPERSCRIPT™ III First-Strand Synthesis System (Invitrogen) were used to synthesize the first strand cDNA. For the cDNA obtained from the immune system of chickens, Expand High Fidelity PCR System (Roche Molecular Systems, IN, USA) was used to produce a single chain variable region library. In each reaction, 1 μL of cDNA, 60 pmol of each primer, 10 μL of 10× reaction buffer solution, 8 μL of 2.5 mM dNTP (Promega, Madison, WI, USA), and 0.5 μL of Taq DNA polymerase were mixed with water. The final volume was 100 μL PCR reaction was performed using the following conditions: 30 cycles of (i) 15 seconds at 94° C. (ii) 30 seconds at 56° C., and (iii) 90 seconds at 72° C., followed by a final extension for 10 minutes at 72° C. The PCR products comprising a fragment having a length of about 350 bp were loaded onto a 1.5% agarose gel and after electrophoresis, QIAGEN Gel II Extraction Kit (QIAGEN, Valencia, CA, USA) was used to purify the nucleotide fragment. The purified PCR product was quantified by reading at OD 260 nm. (1 unit OD=50 μg/ml).

Two VH and VL first products from the second PCR were connected randomly by the overlap extension PCR (Overlap extension PCR). Each PCR reaction was mixed with 100 ng of the purified VL and VH products, 60 pmol of each primer, 10 μL 10× reaction buffer, 8 μL of 2.5 mM dNTP, 0.5 μL of Taq DNA polymerase, and water in a final volume of 100 μL of PCR was performed under the following conditions: 25 cycles of (i) 15 seconds at 94° C., (ii) 30 seconds at 56° C., and (iii) 2 minutes at 72° C., followed by final extension for 10 minutes at 72° C. The PCR products comprising a single chain variable region fragment having a length of about 700 bp were loaded onto a 1.5% agarose gel and after electrophoresis, QIAGEN II Gel Extraction Kit (QIAGEN) was used to purify the nucleotide fragment. The purified PCR product was quantified by reading at OD 260 nm. (1 unit OD=50/ml).

The scFv fragment of the PCR product and vector pComb3X-SS (The Scripps Research Institute, CA, USA) were digested with a Sfi I restriction enzyme. 10 g of the purified overlapping PCT product was mixed with 360 units of Sif I, (μg DNA per 16 units, Roche Molecular Systems, Pleasanton, CA, USA), 20 μL of a 10× reaction buffer, and water to the final volume with 200 μL. 20 g of the pComb3X-SS vector was mixed with 120 units of Sfi I (g DNA per 6 units), 20 μL of a 10× reaction buffer solution, and water to the final volume to 200 μL. The mixture was digested at 50° C. for 8 hours. Afterwards, the digested product comprising the scFv fragment (about 700 bp) and the vector (about 3400 bp) was loaded onto a 1% agarose gel and purified using a Gel Extraction Kit II QIAGEN (QIAGEN, Valencia, CA, USA). 1400 ng of the Sfi I-restricted pComb3X vector and 700 ng of the digested scFv fragments were mixed with 5× a ligase buffer, 10 μL of T4 DNA ligase (Invitrogen, Carlsbad, CA, USA), and water to a final volume of 200 μL. The mixture was incubated at 16° C. for 16 hours to perform the ligation.

After precipitation with ethanol, the DNA pellet was dissolved in 15 μL of water. To produce a library, the ligation sample was transformed into E. coli strain ER2738 (New England Biolabs Inc., Hitchin, Hertfordshine, SG4 OTY, England, UK) via electroporation using the vibrator gene (Gene pulser: Bio-Rad Laboratories, Hercules, CA, USA). Cells were mixed in a 5 ml Super Broth (SB) medium and incubated while stirring at 250 rpm for one hour at 37° C. Then, 3 μL of 100 mg/mL kanamycin was added to 10 mL of SB medium. To determine the library size, 0.1 μL, 1 μL and 10 μL of the culture sample were smeared onto Luria Broth (LB) agar plates containing 50 μg/mL of kanamycin. After stirring for 1 hour, 4.5 μL of 100 mg/mL kanamycin was added to the LB culture and further stirred for an additional 1 hour. Then, 2 ml of the VCM13 helper phage in water (>10¹¹ cfu/ml) was added to the LB medium, along with pre-heated LB (183 mL) containing 92.5 μL of 100 mg/mL kanamycin. This mixture was stirred at 250 rpm at 37° C. for an additional 2 hours. Next, 280 μL (50 mg/mL) of kanamycin was added to the culture and stirred overnight at 37° C. The next day, the bacteria pellet was centrifuged using a high-speed centrifuge (Beckman, JA-10 rotor) at 3,000 g, 4° C. Afterwards, the bacterial pellet was used to extract phagemid DNA, while the supernatant was transferred to sterile centrifuge bottles. Next 8 grams of polyethylene glycol-8000 (PEG-8000, Sigma) and 6 grams of sodium chloride was added (NaCl, Merck) to the supernatant, and then kept for 30 minutes in ice. Afterwards, the supernatant was centrifuged 15 minutes at 15,000 g, 4° C. The supernatant was then discarded, and the phage pellet Tris containing 1% BSA—reproduction was suspended in buffered saline (TBS).

### Example 3

### Library Panning (Bio-Panning) on an Immobilized Antigen

Bio-panning was performed using magnetic beads (Dynabeads M-270 Epoxy, Invitrogen). At room temperature, approximately 1×10⁷ beads were coated with 5 μg of recombinant FAM19A5 protein by stirring, while rotating, the beads and the protein together for 20 hours at room temperature. Once the coating was done, the beads were washed 4 times with phosphate buffered saline (PBS) and blocked for one hour in PBS containing 3% BSA at room temperature. Then, the coated beads were cultured for two hours at room temperature with Phage-displayed scFv described above. To remove any phage that was not bound to the antigen coated beads, the beads were washed with 0.05% Tween20/PBS. Then the bound phages were eluted with 50 μL of 0.1M glycine/hydrogen chloride (0.1M Glycine-HCl, pH 2.2) and neutralized with 3 μL of 2M Tris with hydrogen chloride (tris-HCl, pH 9.1). This phage-containing supernatants were used to infect E. coli ER2738 cells and VCSM13 helper phage was used to amplify and rescue overnight. Also the input (input) and production (output) by phage titers from the phage-infected cultures were determined by blotting the phage-infected cultures on LB agar plates containing 50 μg/ml of kanamycin. The next day, PEG-8000 and NaCl were used to precipitate phages, which were used subsequently for bio-panning. Bio-panning was performed up to a total of five different times by repeating the above process. With each amplification, the phages were screened and selected for high affinity to the FAM19A5 protein.

### Example 4

### Selection of Clone by Phage ELISA

To analyze the clones selected from the bio-panning, we randomly selected individual clones from the phase-displayed scFv and confirmed using ELISA that the clones bind to the FAM19A5 recombinant protein. The FAM19A5 recombinant protein was diluted in 0.1M NaHCO₃ buffer, and 100 ng/well of the protein was used to coat 96-well microtiter plates at 4° C. for 16 hours. Next day, the plates were blocked with 3% BSA/PBS at 37° C. for 1 hour. Then, the phage supernatant was mixed with 6% BSA/PBS and was cultured for 2 hours at 37° C. The plates containing the supernatant were then washed with 0.05% Tween20/PBS. The HRP-conjugated M13 antibody (a-M13-HRP, Pierce Chemical Co, Rockford, IL, USA) was diluted to 1/5000. 50 μL of the diluted antibody was added to the plates and incubated for 1 hour at 37° C. After the incubation and washing, the plates were added with 0.05M citrate buffer solution, 1 μg/ml of 2,2′-azino-bis (3-ethylbenzothiazoline-6-sulphonic acid) (ABTS, Amresco, Solon, OH, USA), and 0.1% H₂O₂ for color development. The absorbance for each well was measured at 405 nm.

Clones (n=24) that bind to the FAM19A5 recombinant protein and show high absorbance were analyzed. From these clones, 13 scFv clones having unique sequences were obtained. After further selection, clones 2-13, 3-2, 1-65, and 1-28 were obtained.

### Example 5

### Production of Anti-FAM19A5-IGG2/4 Antibody

Anti-FAM19A5 scFv was subcloned into a mammalian expression vector In the FAM19A5 scFv gene sequence, a human Cκ gene was connected to the light chain variable domain, and human immunoglobulin isotype IgG2/4 of CH1, CH2, and CH3 genes were connected to the heavy chain variable region. The antibody having each light chain and each heavy chain was synthesized by adding restriction sites (Genscript, USA). The synthesized gene was inserted into the mammalian cell expression vector having a modified restriction site to facilitate cloning. First, the light chain gene was inserted into the vector using Hind III and Xba I (New England Biolabs, UK) restriction enzymes and then adding the heavy chain gene to the vector by using NheI and BamHI (New England Biolabs, UK) restriction enzymes.

In order to express and purify an anti-FAM19A5-IgG2/4 antibody, we used a mammalian cell transfection and overexpression injection system. We mixed 2 μg/ml of the mammalian expression vector with 4 μg of polyethyleneimine (PEI, Polysciences, Warrington, PA, USA) in 150 mM sodium chloride (NaCl, Merck) corresponding to 1/10of the cell culture volume. The mixture was allowed to stand for 15 minutes at room temperature. The mixture was added to HEK293F cells (2×10⁶ cells/ml, Invitrogen), which were then incubated in the FREESTYLE™ 293 expression culture medium containing 100 U/ml of penicillin and streptomycin (Invitrogen) at 7% C02 and 37° C. and in a stirring condition of 135 rpm for six days. To purify the expressed anti-FAM19A5 IgG2/4 antibodies from the cell culture supernatant, we used Protein A bead (RepliGen, Waltham, MA, USA) affinity gel chromatography. The protein A chromatography was run on 4˜12% Bis-Tris gradient gel electrophoresis. The size and yield of the protein was confirmed by the Coomassie Brilliant Blue staining.

### Example 6

### Comparison of the Demographic and Clinical Characteristics and Serum FAM19A5 Protein Concentrations

### Participants

In order to begin assessing the levels of FAM19A5 protein in patients with a mood disorder, a total of 110 patients diagnosed with major depressive disorder (MDD) and a total of 43 patients diagnosed with bipolar I (n=26) or II (n=17) disorder (BD) were recruited from the outpatient psychiatric clinic of Korea University Anam Hospital (Seoul, Republic of Korea). The average age of the patients ranged from 20-65 years and were diagnosed with either MDD or BD by two independent psychiatrists. For the control group, a total of 112 healthy volunteers (20-65 years of age with no history of psychiatric disorders) were recruited from the local community.

The initial diagnosis of MDD and BD was based on the Diagnostic and Statistical Manual of Mental Disorders (DSM)-IV criteria. This diagnosis was confirmed by a psychiatrist using the Structured Clinical Interview for DSV-IV Axis I disorders (SCID-I). The duration of the illness, including major depressive, manic, or hypomani episodes, was assessed in an interview using the life-chart methodology. The depressive symptoms of all participants were evaluated using the 17-item Hamilton Depression Rating Scale (HDRS) on the day of the MRI scan. Hamilton M., J Neurol Neurosurg Psychiatry 23:56-62 (1960).

The exclusion criteria for the MDD group were as follows: (1) primary comorbid diagnosis of any other major Axis I or Axis II psychiatric illness within the last 6 months; (2) MDD with psychotic features; (3) acute suicidal or homicidal patients requiring inpatient treatment; (4) history of a serious or unstable medical illness; (5) primary neurological illness; and (6) any contraindication for magnetic resonance imaging (MRI). The exclusion criteria for the BD group were the same as those used in the MDD group, except for the use of the psychotic features.

At study enrolment, 58 MDD patients were medication-naïve (determined as drug-naïve MDD group) and 52 MDD patients were being treated with antidepressants (determined as medicated-MDD group). All patients with BD were taking psychotropic medication. All participants were right-handed according to the Edinburgh Handedness Test. Oldfield R. C., Neuropsychologia 9:97-113 (1971). The study protocol was approved by the Institutional Review Board of the Korea University Anam Hospital in accordance with the Declaration of Helsinki (revised in 2008), and all participants provided written informed consent prior to participation.

### Statistical Analysis

With regard to the comparison of serum FAM19A5 concentration among subgroups, analysis of covariance (ANCOVA) with individual cortical thickness as dependent variables; subgroups as independent variable; and age and sex as covariates. In regard to the cortical thickness analysis, the data of 51 drug-naïve and 51 medicated patients with MDD, 28 BD patients, and 92 healthy controls were included. The correlation between the cortical thickness and serum FAM19A5 concentration was analyzed using a two-tailed Pearson's partial correlation, separately for following each group determined by diagnosis and medication status: MDD group, BD group (no BD patients were drug-naïve), HC group, drug-naïve MDD group, and medicated-MDD group. In the Pearson's partial correlation analysis between serum FAM19A5 concentration and cortical thickness, age, sex, HDRS score, and medication status (only for MDD group) were adjusted as covariates. Furthermore, we applied Fisher's r-to-z transformation to compare the obtained correlation coefficients of any significant correlation (P<0.05) between the groups as follows: drug-naïve MDD group vs. HC group; medicated-MDD group vs. HC group; BD group vs. HC group. We also performed the comparison of the cortical thickness between groups using an ANCOVA adjusted for age, sex, and medication (only for MDD group vs. HC group) as nuisance covariates. For the multiple comparison correction, the false discovery rate (FDR) was applied in the correlation analyses for multiple comparison correction, q<0.05; 76 comparisons in both hemispheres (Benjamini and Hochberg, 1995). We also performed a receiver operating characteristic (ROC) curve analysis to test whether serum FAM19A5 concentration, as a screening tool, can accurately discriminate patients with MDD or BD from healthy controls or BD patients from MDD patients. The area under the curve (AUC) was used as a measure of the dichotomous screening ability of the test. Screening ability was computed between drug-naïve MDD group vs. HC group, between medicated MDD group vs. HC group, between BD group vs. HC group, drug-naïve MDD group vs. BD group, and between medicated MDD group vs. BD group. The corresponding sensitivity, specificity, accuracy, and AUC were subsequently calculated. To analyze group differences due to demographic and clinical characteristics, age, HDRS score, and duration of illness were analyzed using analysis of variance (ANOVA), and the distributions of sex was analyzed using chi-squared tests. All statistical analyses were performed using SPSS version 18.0 (SPSS Inc., Chicago, IL, USA).

### Results

Table 8 (below) provides a comparison of the age, gender, HDRS-17 score, duration of illness (in months), number of drug-naïve or medicated patients, and serum FAM19A5 protein concentration (ng/mL) of the participants from each of the groups—i.e., healthy individuals (HC), non-medicated MDD patients (DN-MDD), medicated MDD patients (M-MDD), and non-medicated bipolar I or II disorder patients (BD). Significant differences were observed for age (F_{(1,264)}=7.391, P<0.001), sex (χ²=22.060, P=0.004), HDRS score (F_{(1,264)}=118.075, P<0.001), and duration of illness (F_{(1,264)}=7.391, P=0.004).

TABLE 8
Demographic and clinical characteristics of major depressive
disorder, bipolar disorder, and healthy control groups

| | HC | DN-MDD | M-MDD | BD | p value |
|---|---|---|---|---|---|
| Characteristics | (n = 112) | (n = 58) | (n = 52) | (n = 43) | (F, χ²) |
| Age | 39.83 ± 13.23 | 41.93 ± 11.90 | 46.50 ± 11.69 | 34.81 ± 11.61 | <0.001 |
| | | | | | (F = 7.391) |
| Gender | 75/37 | 42/16 | 42/10 | 16/27 | 0.004 |
| (female/male) | | | | | (χ² = 22.060) |
| HDRS-17 score | 1.92 ± 2.02 | 17.10 ± 6.75 | 9.46 ± 7.66 | 5.23 ± 4.27 | <0.001 |
| | | | | | (F = 118.075) |
| Duration of | n.a. | 27.76 ± 41.34 | 61.85 ± 47.76 | 51.05 ± 71.70 | 0.004 |
| illness (months) | | | | | (F = 5.839) |
| Drug- | 110/0 | 58/0 | 0/52 | 0/43 | n.a. |
| naïve/medicated | | | | | |
| Serum FAM19A5 | 1.03 ± 0.30 | 2.05 ± 1.14 | 1.10 ± 0.29 | 2.37 ± 0.60 | <0.001 |
| (ng/mL) | | | | | (F = 35.008) |

Data represent mean ± standard error for age, HDRS-17 scores, duration of illness, and serum FAM19A5 concentration.
The p value for distribution of sex was obtained by chi-square test.
The p values for comparisons of age, HDRS-17 score, and duration of illness were obtained by analysis of variance (ANOVA).
The p values for comparisons of serum FAM19A5 was obtained by analysis of covariance (ANCOVA) adjusting for age and sex.
MDD, major depressive disorder; HC, healthy controls; BD, bipolar disorder patients; HDRS-17, Hamilton Depression Rating Scale.

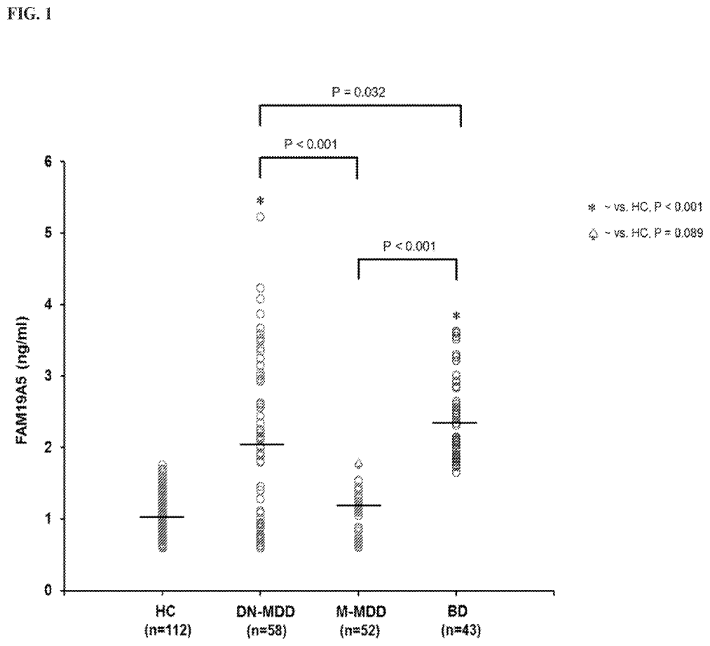

Significant differences were also observed in the serum FAM19A5 protein concentrations among the four groups. See Table 9 (above) and FIG. 1. Compared to the HC group, both the drug-naïve MDD group (F_{(1,169)}=76.620, P<0.001) and the BD group (F_{(1,154)}=302.718, P<0.001) showed significantly higher FAM19A5 protein concentration in the serum. The drug-naïve group (F_{(1,109)}=36.912, P<0.001) and the BD group (F_{(1,94)}=139.808, P<0.001) also had significantly higher serum FAM19A5 protein levels than the medicated-MDD group. Between the drug-naïve MDD group and the BD group, the BD group had higher concentration of FAM19A5 protein in the serum.

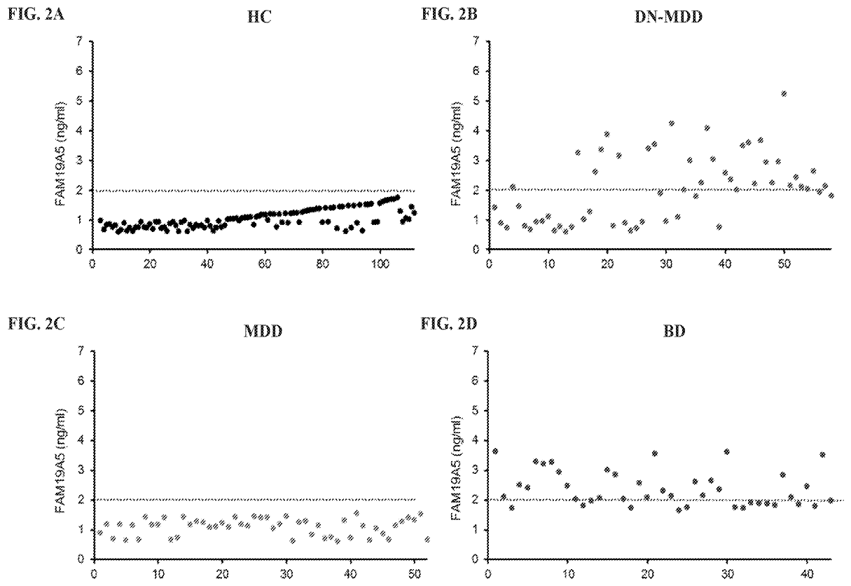

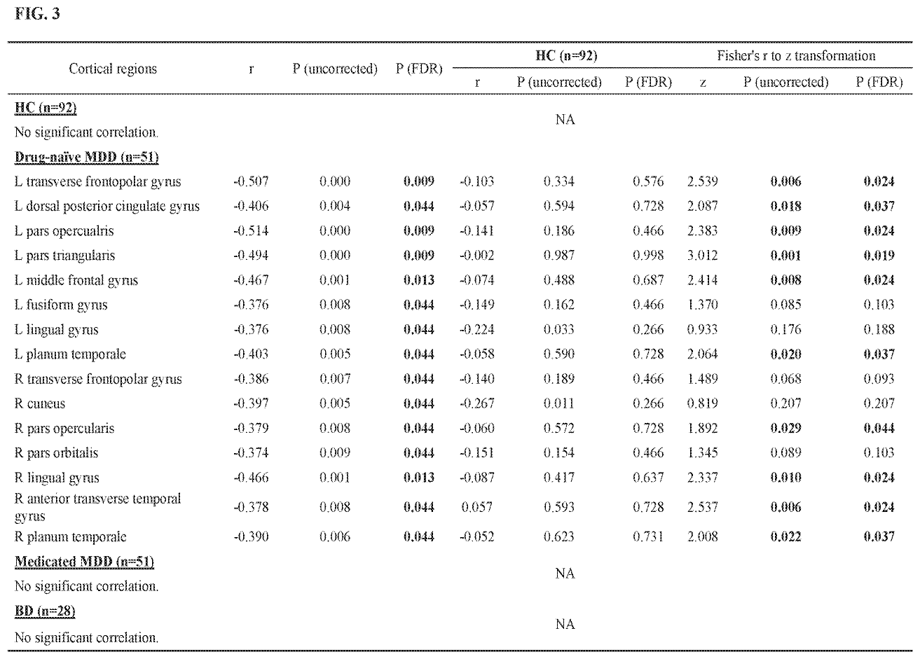

No significant differences in the serum FAM19A5 protein concentrations were observed between the medicated-MDD group and the HC group (F(1,163)=2.936, P=0.089). Scatter plots representing the distribution of serum FAM19A5 concentrations from the different groups are provided in FIGS. 2A to 2D.

### Example 7

### Comparison of Cortical Thickness and Serum FAM19A5 Protein Concentrations

### MRI Data Acquisition

In order to assess the association between cortical thickness and serum FAM19A5 protein concentrations among the different diagnostic groups described above (Example 6), T1-weighted images of the brains of the participants were obtained using a 3.0 T Siemens Trio whole-body imaging system (Siemens Medical Systems, Iselin, New Jersey, United States). The T1-weighted images were acquired parallel to the anterior-commissure-posterior-commissure line using the 3D T1-weighted magnetization-prepared rapid gradient-echo (MP-RAGE) sequence with the following parameters: repetition time (TR), 1900 ms; echo time (TE), 2.6 ms; field of view, 220 mm; matrix size, 256×256; slice thickness, 1 mm; number of coronal slices, 176 (without gap); voxel size, 0.86×0.86×1 mm³; flip angle, 160 flip angle; and number of excitations, 1.

### Image Processing

Then, the cortical thickness was calculated from the T1-weighted images using the automated procedures implemented in the FreeSurfer 5.3 development version (Massachusetts General Hospital, Boston, U.S., surfer.nmr.mgh.harvard.edu). To do so, a three-dimensional model of cortical surface reconstructions was computed using the T1-weighted images in the FreeSurfer program. Details of the technical aspects of these procedures have been described in previous publications. See Dale A. M. et al., Neuroimage 9:179-94 (1999); Fischl B. et al., IEEE Trans Med Imaging 20:70-80 (2001); Fischl B. et al., Neuron 33:341-55 (2002); Fischl B. et al., Neuroimage 9:195-207 (1999); Fischl B. et al., Cereb Cortex 14:11-22 (2004); and Segonne F. et al., IEEE Trans Med Imaging 26:518-29 (2007).

Briefly, the following steps were carried out (Han K. M. et al., Sci Rep. 7:42621 (2017)) removal of non-brain tissue, automated Talairach transformation of each subject's native brain, segmentation of the volumetric structures, inflation of the cortical surface to an average spherical surface, intensity normalization, and automated topology correction. The transition between gray/white matter and the pial boundary were determined by detecting the greatest shift in intensity through surface deformation. The entire cortex of each subject was then visually inspected, and data from subjects with inaccuracies in segmentation were discarded. Each hemisphere was then automatically parcellated into 74 distinct cortical regions consisting of gyri and sulci by a previously described method (Han et al., 2017), and thickness of these cortical regions were automatically calculated. Cortical thickness from 38 cortical gyri in each hemisphere were included in the analysis.

### Results

As shown in FIG. 3, in the drug-naïve MDD group, there was a statistically significant inverse relationship between the thickness of 15 cortical gyri (both prefrontal cortex, posterior cingulate cortex, superior temporal region, occipital regions, both pars opercularis and planum temporale, left transverse frontopolar and dorsal posterior cingulate gyrus, pars triangularis, middle frontal gyrus, right lingual gyrus, and anterior transverse temporal gyrus) and serum FAM19A5 protein concentrations (all, FDR-corrected P [P_{FDR}]<0.05). In the HC group, no such relationship was observed in these cortical gyri (all, P_{FDR}>0.05). There was also no significant relationship between cortical thickness and serum FAM19A5 protein concentrations among participants from the medicated-MDD group or the BD group (all, P_{FDR}>0.05).

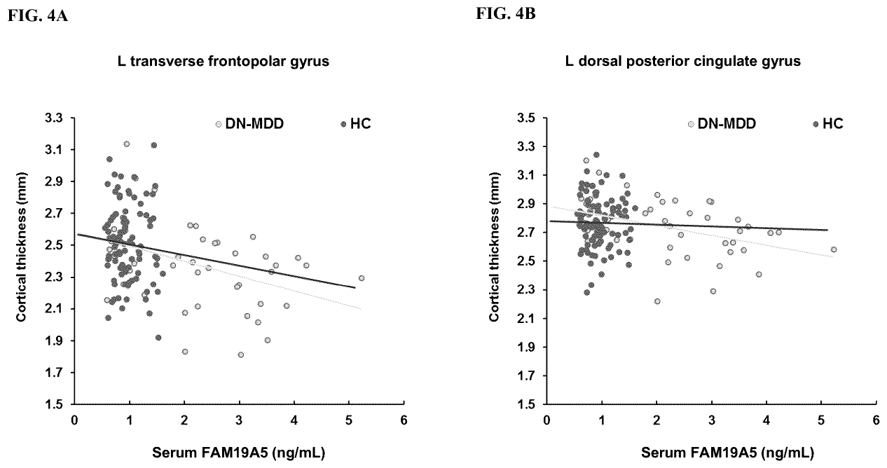

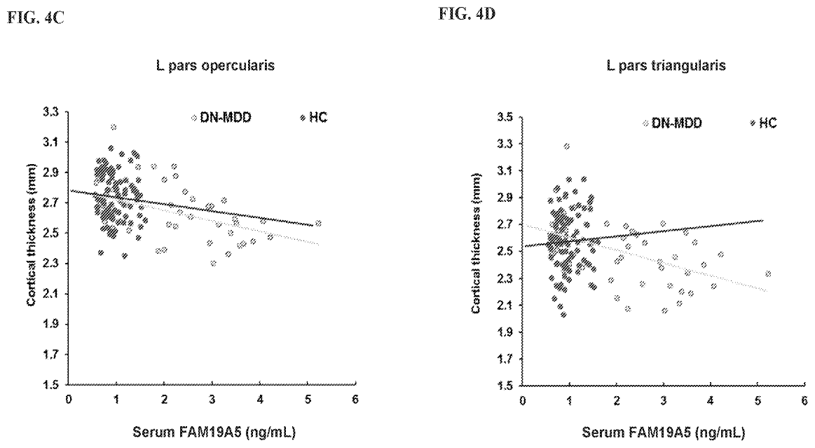

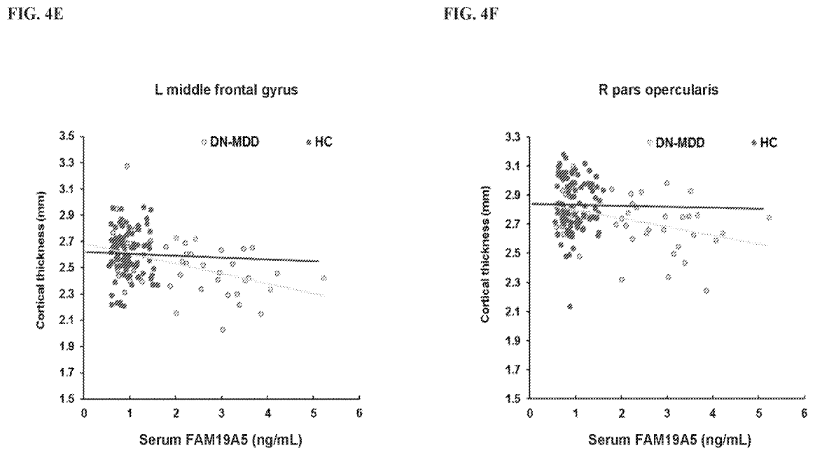

Additionally, when compared to the corresponding cortical regions in the HC group, the correlation coefficients between serum FAM19A5 protein concentrations and the cortical thickness of certain brain regions (i.e., both pars opercularis and planum temporale, left transverse frontopolar and dorsal posterior cingulate gyrus, pars triangularis, middle frontal gyrus, and right lingual and anterior transverse temporal gyrus) were significantly different. FIGS. 4A to 4E provides scatter plots showing the relationship between cortical thickness and serum FAM19A5 concentrations from some of these cortical regions.

### Example 8

### ROC Curve Analysis of Serum FAM19A5 Protein Concentration for the Diagnosis of Mood Disorders

To further assess the capability of measuring FAM19A5 protein levels in the serum to diagnose mood disorders (e.g., major depressive disorder or bipolar disorder), a receiver operating characteristic curve (ROC curve) analysis was performed for the different comparison groups. The comparison groups included: (i) drug-naïve MDD vs. HC; (ii) medicated MDD vs. HC; (iii) BD vs. HC; (iv) BD vs. drug-naïve MDD; and (v) BD vs. medicated MDD.

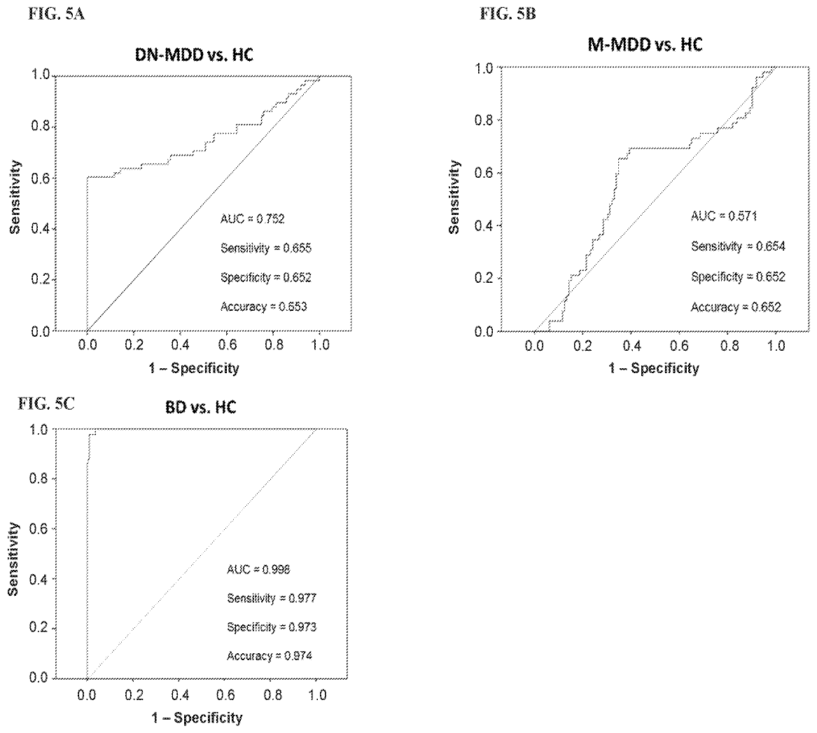

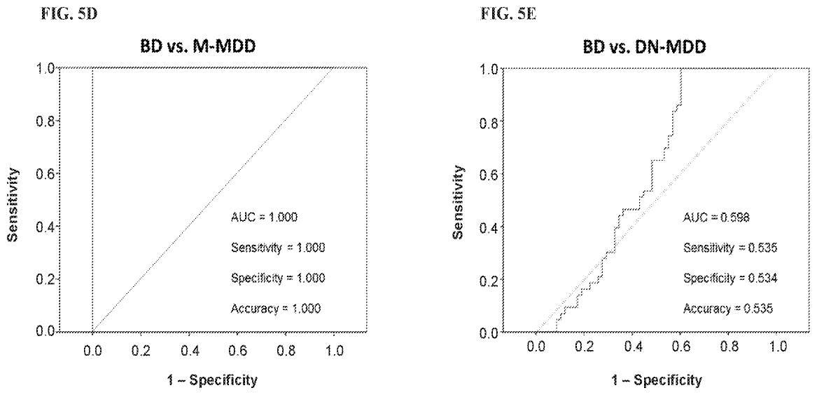

As shown in FIGS. 5C and 5D, the analysis between BD vs. HC and BD vs. M-MDD suggests that serum FAM19A5 protein concentrations is likely a good indicator for the diagnosis of bipolar I or II disorder. The relevant values for the BD vs. HC analysis include: AUC: 0.998, sensitivity: 0.977, specificity: 0.973, accuracy: 0.974. The relevant values for the BD v. M-MDD include: AUC: 1.000, sensitivity: 1.000, specificity: 1.000, accuracy: 1.000. Also, as shown in FIG. 5A, serum FAM19A5 protein concentration may also be effective in diagnosing major depressive disorder (AUC: 0.752, sensitivity: 0.655, specificity: 0.652, accuracy: 0.653). Not surprisingly, as shown in FIGS. 5B and 5E, serum FAM19A5 protein concentrations are not likely effective in distinguishing between M-MDD vs. HC (AUC: 0.571, sensitivity: 0.654, specificity: 0.652, accuracy: 0.652) and BD vs. DN-MDD (AUC: 0.598, sensitivity: 0.535, specificity: 0.534, accuracy: 0.535).

Collectively, the above results demonstrate that measuring serum FAM19A5 protein concentrations in patients and comparing to concentrations from healthy individuals may be an effective means of diagnosing mood disorders (e.g., major depressive disorder or bipolar disorder).

### Example 9

### Evaluation of Response to Acute Stress after In Vivo Administration of Anti-FAM19A5 Antibody

To assess the efficacy of anti-FAM19A5 antibodies in treating mood disorders, particularly major depressive disorder, a rat model of depression will be used, in which the FAM19A5 antibodies will be administered to the rats and their response to acute stress will be assessed using the forced swim test (FST) as described in Porsolt R. D. et al., Nature 266:730-732 (1977).

Briefly, rats will be divided into three different groups and each of the group will receive (i) no treatment (i.e., negative control), (ii) anti-FAM19A5 antibodies (i.e., experimental group), or (iii) known antidepressant (i.e., positive control) for approximately a week. Afterwards, the rats from each of the groups will be placed in an inescapable cylinder containing water. After an initial period of struggling, swimming, and/or climbing, the rats will eventually display a floating or immobile posture, with their noses held just above water. The duration of time spent as “immobile” (no movement except those necessary to keep the nose above water), “struggling/climbing” (quick movements of the forelimbs observed such that the front paws break the surface of the water), and “swimming” (movement of forelimbs or hind limbs in a paddling fashion observed) will be compared among the groups. Upon completion of the forced swim test, peripheral blood will also be collected from the animals to measure their serum FAM19A5 protein levels. It is predicted that rats treated with the known antidepressant or the anti-FAM19A5 antibodies will spend more time “struggling/climbing” or “swimming” compared to the non-treated rats. It is also predicted that the serum FAM19A5 protein levels will be lower in rats treated with the known antidepressant or the anti-FAM19A5 antibodies compared to the non-treated animals.

It is to be appreciated that the Detailed Description section including the Summary and Abstract sections is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

This PCT application claims the priority benefit of U.S. Provisional Application Nos. 62/566,877, filed Oct. 2, 2017, and 62/582,888, filed Nov. 7, 2017, both of which are incorporated herein by reference in their entireties.

1. A method of determining a FAM19A5 protein level or a FAM19A5 mRNA level in a subject having a mood disorder comprising detecting the FAM19A5 protein level or FAM19A5 mRNA level from a biological sample obtained from the subject compared to a corresponding level from a normal control.

2. (canceled)

3. The method of claim 1, wherein the FAM19A5 protein level or the FAM19A5 mRNA level from the biological sample is higher compared to the corresponding level from the normal control.

4. The method of claim 1, wherein (i) the FAM19A5 protein level is measured by an immunohistochemistry, a Western blotting, a radioimmunoassay, an enzyme linked immunosorbent assay (ELISA), a radioimmunodiffusion, an immunoprecipitation assay, an Ouchterlony immunodiffusion method, a rocket Immunoelectrophoresis, a tissue immunostaining method, a complement fixation assay, FACS, a protein chip, or any combination thereof; or (ii) the FAM19A5 mRNA level is measured by a RT-PCR, a real time polymerase chain reaction, a Northern blot, or any combination thereof.

5. (canceled)

6. The method of claim 1, wherein the biological sample comprises a tissue, cell, blood, serum, plasma, saliva, urine, cerebrospinal fluid (CSF), or any combination thereof.

7. A method of treating a mood disorder in a subject in need thereof comprising administering to the subject an antagonist against a family with sequence similarity 19, member A5 (FAM19A5) protein (“FAM19A5 antagonist”).

8. A method of increasing thickness of a cortical gyrus in a subject having a mood disorder comprising administering to the subject an antagonist against a family with sequence similarity 19, member A5 (FAM19A5) protein (“FAM19A5 antagonist”).

9. The method of claim 8, wherein the cortical gyrus comprises a transverse frontopolar gyrus, dorsal posterior cingulate gyrus, pars opercualris, pars triangularis, middle frontal gyrus, fusiform gyrus, lingual gyrus, planum temporale, cuneus, pars orbitalis, anterior transverse temporal gyrus, planum temporale, or any combinations thereof.

10. The method of claim 7, wherein the mood disorder comprises a major depressive disorder (MDD), bipolar disorder (BD), minor depressive disorder, persistent depressive disorder (dysthymia), seasonal affective disorder (SAD), psychotic depression, postpartum depression, premenstrual dysphoric disorder (PMDD), situational depression, atypical depression, anxiety disorder, or any combination thereof.

11. The method of claim 7, wherein the FAM19A5 antagonist is (i) an antisense oligonucleotide, an siRNA, an shRNA, a miRNA, a dsRNA targeting FAM19A5, an aptamer, a PNA, a vector including the same, or any combinations thereof, (ii) an antibody that specifically binds to FAM19A5 protein (“anti-FAM19A5 antibody”), (iii) a polynucleotide encoding the anti-FAM19A5 antibody, or (iv) combinations thereof.

12. (canceled)

13. The method of claim 11, wherein the anti-FAM19A5 antibody comprises a heavy chain CDR1, CDR2, and CDR3, and a light chain CDR1, CDR2, and CDR3, wherein
(i) the heavy chain CDR1, CDR2, and CDR3 comprises the amino acid sequences set forth in SEQ ID NOs: 11, 12, and 13, respectively, and the light chain CDR1, CDR2, and CDR3 comprises the amino acid sequences set forth in SEQ ID NOs: 23, 24, and 25, respectively;
(ii) the heavy chain CDR1, CDR2, and CDR3 comprises the amino acid sequences set forth in SEQ ID NOs: 14, 15, and 16, respectively, and the light chain CDR1, CDR2, and CDR3 comprises the amino acid sequences set forth in SEQ ID NOs: 26, 27, and 28, respectively;
(iii) the heavy chain CDR1, CDR2, and CDR3 comprises the amino acid sequences set forth in SEQ ID NOs: 17, 18, and 19, respectively, and the light chain CDR1, CDR2, and CDR3 comprises the amino acid sequences set forth in SEQ ID NOs: 29, 30, and 31, respectively; or
(iv) the heavy chain CDR1, CDR2, and CDR3 comprises the amino acid sequences set forth in SEQ ID NOs: 20, 21, and 22, respectively, and the light chain CDR1, CDR2, and CDR3 comprises the amino acid sequences set forth in SEQ ID NOs: 32, 33, and 34, respectively.

14. The method of claim 11, wherein the anti-FAM19A5 antibody comprises a heavy chain variable region (VH) and a light chain variable region (VL), wherein
(i) the VH comprises the amino acid sequence set forth in SEQ ID NO: 35 and the VL comprises the amino acid sequence set forth in SEQ ID NO: 39;
(ii) the VH comprises the amino acid sequence set forth in SEQ ID NO: 36 and the VL comprises the amino acid sequence set forth in SEQ ID NO: 40;
(iii) the VH comprises the amino acid sequence set forth in SEQ ID NO: 37 and the VL comprises the amino acid sequence set forth in SEQ ID NO: 41; or
(iv) the VH comprises the amino acid sequence set forth in SEQ ID NO: 38 and the VL comprises the amino acid sequence set forth in SEQ ID NO: 42.

15. The method of claim 11, wherein the anti-FAM19A5 antibody comprises a heavy chain variable region (VH) and a light chain variable region (VL), wherein the VH comprises an amino acid sequence which is at least about 80% identical to the amino acid sequence set forth in SEQ ID NO: 35, 36, 37, or 38; and wherein the VL comprises an amino acid sequence which is at least about 80% identical to the amino acid sequence set forth in SEQ ID NO: 39, 40, 41, or 42.

16. (canceled)

17. The method of claim 1, further comprising administering to the subject an anti-depressant.

18. The method of claim 17, wherein the anti-depressant comprises (i) an antisense oligonucleotide, an siRNA, an shRNA, a miRNA, a dsRNA targeting FAM19A5, an aptamer, a PNA, a vector including the same, or any combinations thereof; (ii) an antibody that specifically binds to FAM19A5 protein (“anti-FAM19A5 antibody”); (iii) a polynucleotide encoding the anti-FAM19A5 antibody; or (iv) combinations thereof.

19. The method of claim 1, wherein the detecting comprises contacting a FAM19A5 antagonist with a biological sample of the subject and measuring the FAM19A5 protein level or FAM19A5 mRNA level in the sample.

20. The method of claim 19, wherein the FAM19A5 antagonist comprises (i) an antisense oligonucleotide, an siRNA, an shRNA, a miRNA, a dsRNA targeting FAM19A5, an aptamer, a PNA, a vector including the same, or any combinations thereof; (ii) an antibody that specifically binds to FAM19A5 protein (“anti-FAM19A5 antibody”); (iii) a polynucleotide encoding the anti-FAM19A5 antibody; or (iv) combinations thereof.

21. The method of claim 8, wherein the FAM19A5 antagonist comprises (i) an antisense oligonucleotide, an siRNA, an shRNA, a miRNA, a dsRNA targeting FAM19A5, an aptamer, a PNA, a vector including the same, or any combinations thereof; (ii) an antibody that specifically binds to FAM19A5 protein (“anti-FAM19A5 antibody”); (iii) a polynucleotide encoding the anti-FAM19A5 antibody; or (iv) combinations thereof.

22. The method of claim 18, wherein the anti-FAM19A5 antibody comprises a heavy chain CDR1, CDR2, and CDR3, and a light chain CDR1, CDR2, and CDR3, wherein
(i) the heavy chain CDR1, CDR2, and CDR3 comprises the amino acid sequences set forth in SEQ ID NOs: 11, 12, and 13, respectively, and the light chain CDR1, CDR2, and CDR3 comprises the amino acid sequences set forth in SEQ ID NOs: 23, 24, and 25, respectively;
(ii) the heavy chain CDR1, CDR2, and CDR3 comprises the amino acid sequences set forth in SEQ ID NOs: 14, 15, and 16, respectively, and the light chain CDR1, CDR2, and CDR3 comprises the amino acid sequences set forth in SEQ ID NOs: 26, 27, and 28, respectively;
(iii) the heavy chain CDR1, CDR2, and CDR3 comprises the amino acid sequences set forth in SEQ ID NOs: 17, 18, and 19, respectively, and the light chain CDR1, CDR2, and CDR3 comprises the amino acid sequences set forth in SEQ ID NOs: 29, 30, and 31, respectively; or
(iv) the heavy chain CDR1, CDR2, and CDR3 comprises the amino acid sequences set forth in SEQ ID NOs: 20, 21, and 22, respectively, and the light chain CDR1, CDR2, and CDR3 comprises the amino acid sequences set forth in SEQ ID NOs: 32, 33, and 34, respectively.

23. The method of claim 20, wherein the anti-FAM19A5 antibody comprises a heavy chain CDR1, CDR2, and CDR3, and a light chain CDR1, CDR2, and CDR3, wherein
(i) the heavy chain CDR1, CDR2, and CDR3 comprises the amino acid sequences set forth in SEQ ID NOs: 11, 12, and 13, respectively, and the light chain CDR1, CDR2, and CDR3 comprises the amino acid sequences set forth in SEQ ID NOs: 23, 24, and 25, respectively;
(ii) the heavy chain CDR1, CDR2, and CDR3 comprises the amino acid sequences set forth in SEQ ID NOs: 14, 15, and 16, respectively, and the light chain CDR1, CDR2, and CDR3 comprises the amino acid sequences set forth in SEQ ID NOs: 26, 27, and 28, respectively;
(iii) the heavy chain CDR1, CDR2, and CDR3 comprises the amino acid sequences set forth in SEQ ID NOs: 17, 18, and 19, respectively, and the light chain CDR1, CDR2, and CDR3 comprises the amino acid sequences set forth in SEQ ID NOs: 29, 30, and 31, respectively; or
(iv) the heavy chain CDR1, CDR2, and CDR3 comprises the amino acid sequences set forth in SEQ ID NOs: 20, 21, and 22, respectively, and the light chain CDR1, CDR2, and CDR3 comprises the amino acid sequences set forth in SEQ ID NOs: 32, 33, and 34, respectively.

24. The method of claim 21, wherein the anti-FAM19A5 antibody comprises a heavy chain CDR1, CDR2, and CDR3, and a light chain CDR1, CDR2, and CDR3, wherein
(i) the heavy chain CDR1, CDR2, and CDR3 comprises the amino acid sequences set forth in SEQ ID NOs: 11, 12, and 13, respectively, and the light chain CDR1, CDR2, and CDR3 comprises the amino acid sequences set forth in SEQ ID NOs: 23, 24, and 25, respectively;
(ii) the heavy chain CDR1, CDR2, and CDR3 comprises the amino acid sequences set forth in SEQ ID NOs: 14, 15, and 16, respectively, and the light chain CDR1, CDR2, and CDR3 comprises the amino acid sequences set forth in SEQ ID NOs: 26, 27, and 28, respectively;
(iii) the heavy chain CDR1, CDR2, and CDR3 comprises the amino acid sequences set forth in SEQ ID NOs: 17, 18, and 19, respectively, and the light chain CDR1, CDR2, and CDR3 comprises the amino acid sequences set forth in SEQ ID NOs: 29, 30, and 31, respectively; or
(iv) the heavy chain CDR1, CDR2, and CDR3 comprises the amino acid sequences set forth in SEQ ID NOs: 20, 21, and 22, respectively, and the light chain CDR1, CDR2, and CDR3 comprises the amino acid sequences set forth in SEQ ID NOs: 32, 33, and 34, respectively.


SEQUENCE LISTING (200 sequences)

SEQ ID NO: 1 - protein, 132 aa, Homo sapiens
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 2 - protein, 125 aa, Homo sapiens
MQLLKALWALAGAALCCFLVLVIHAQFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 3 - protein, 53 aa, Homo sapiens
MYHHREWPARIIKTKQWCDMLPCLEGEGCDLLINRSGWTCTQPGGRIKTTTVS

SEQ ID NO: 4 - DNA, 1501 bp, Homo sapiens
  source 1..1501: mol_type = genomic DNA
ggcggcggaggatggcgcgcgcggggcccgcacgtggaggccggcgcgggggcgcgggcagggccggctgctgagacgcgctgctgccccccgcgcgggcgccgcggcttcaatggcgccatcgcccaggaccggcagccggcaagatgcgaccgccctgcccagcatgtcctcaactttctgggcgttcatgatcctggccagcctgctcatcgcctactgcagtcagctggccgccggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctgacaaacacagcccctgagggggccccgggagtggccttggctccctggagagcccacgtctcagccacagttctccactcgcctcggacttcacccgttctctgccgcccgcccactccgtttccctgtggtccgtgaaggacggcctcaggccttggcatcctgagcttcggtctgtccagccgacccgaggaggccggactcagacacataggcggggggcggcacctggcatcagcaatacgcagtctgtgggagcccggccgcgcccagcccccgccgaccgtggcgttggccctgctgtcctcagaggaggaggaggaggaggcagctccggcagccacagaaggctgcagcccagcccgcctgagacacgacgcctgccccaggggactgtcaggcacagaagcggcctcctcccgtgccccagactgtccgaattgcttttattttcttatactttcagtatactccatagaccaaagagcaaaatctatctgaacctggacgcaccctcactgtcagggtccctggggtcgcttgtgcgggcgggagggcaatggtggcagagacatgctggtggccccggcggagcggagagggcggccgtggtggaggcctccaccccaggagcaccccgcacaccctcggaggacgggcttcggctgcgcggaggccgtggcacacctgcgggaggcagcgacggcccccacgcagacgccgggaacgcaggccgctttattcctctgtacttagatcaacttgaccgtactaaaatccctttctgttttaaccagttaaacatgcctcttctacagctccatttttgatagttggataatccagtatctgccaagagcatgttgggtctcccgtgactgctgcctcatcgataccccatttagctccagaaagcaaagaaaactcgagtaacacttgtttgaaagagatcattaaatgtattttgcaaagcccaaaaaaaaaaaaaaaa

SEQ ID NO: 5 - protein, 14 aa, synthetic construct
  REGION 1..14: note = Epitope F1
QLAAGTCEIVTLDR

SEQ ID NO: 6 - protein, 20 aa, synthetic construct
  REGION 1..20: note = Epitope F2
TLDRDSSQPRRTIARQTARC

SEQ ID NO: 7 - protein, 20 aa, synthetic construct
  REGION 1..20: note = Epitope F3
TARCACRKGQIAGTTRARPA

SEQ ID NO: 8 - protein, 20 aa, synthetic construct
  REGION 1..20: note = Epitope F4
ARPACVDARIIKTKQWCDML

SEQ ID NO: 9 - protein, 20 aa, synthetic construct
  REGION 1..20: note = Epitope F5
CDMLPCLEGEGCDLLINRSG

SEQ ID NO: 10 - protein, 20 aa, synthetic construct
  REGION 1..20: note = Epitope F6
NRSGWTCTQPGGRIKTTTVS

SEQ ID NO: 11 - protein, 5 aa, synthetic construct
  REGION 1..5: note = 2-13 Antibody VH-CDR1
SHGMF

SEQ ID NO: 12 - protein, 17 aa, synthetic construct
  REGION 1..17: note = 2-13 Antibody VH-CDR2
EITNDGSGTNYGSAVKG

SEQ ID NO: 13 - protein, 20 aa, synthetic construct
  REGION 1..20: note = 2-13 Antibody VH-CDR3
STYECPGGFSCWGDTGQIDA

SEQ ID NO: 14 - protein, 5 aa, synthetic construct
  REGION 1..5: note = 3-2 Antibody VH-CDR1
SFNMF

SEQ ID NO: 15 - protein, 17 aa, synthetic construct
  REGION 1..17: note = 3-2 Antibody VH-CDR2
QISSSGSSTNYAPAVRG

SEQ ID NO: 16 - protein, 22 aa, synthetic construct
  REGION 1..22: note = 3-2 Antibody VH-CDR3
SSYDCPYGHCSSGVDSAGEIDA

SEQ ID NO: 17 - protein, 5 aa, synthetic construct
  REGION 1..5: note = 1-65 Antibody VH-CDR1
SYQMG

SEQ ID NO: 18 - protein, 10 aa, synthetic construct
  REGION 1..10: note = 1-65 Antibody VH-CDR2
VINKSGSDTS

SEQ ID NO: 19 - protein, 13 aa, synthetic construct
  REGION 1..13: note = 1-65 Antibody VH-CDR3
GSASYITAATIDA

SEQ ID NO: 20 - protein, 8 aa, synthetic construct
  REGION 1..8: note = 1-28 Antibody VH-CDR1
GFDFSDYG

SEQ ID NO: 21 - protein, 8 aa, synthetic construct
  REGION 1..8: note = 1-28 Antibody VH-CDR2
IRSDGSNP

SEQ ID NO: 22 - protein, 29 aa, synthetic construct
  REGION 1..29: note = 1-28 Antibody VH-CDR3
AKDGNGYCALDAYRSGGYSCGVYPGSIDA

SEQ ID NO: 23 - protein, 8 aa, synthetic construct
  REGION 1..8: note = 2-13 Antibody VL-CDR1
SGGSYSYG

SEQ ID NO: 24 - protein, 7 aa, synthetic construct
  REGION 1..7: note = 2-13 Antibody VL-CDR2
WDDERPS

SEQ ID NO: 25 - protein, 11 aa, synthetic construct
  REGION 1..11: note = 2-13 Antibody VL-CDR3
GTEDISGTAGV

SEQ ID NO: 26 - protein, 13 aa, synthetic construct
  REGION 1..13: note = 3-2 Antibody VL-CDR1
SGGGSYAGSYYYG

SEQ ID NO: 27 - protein, 7 aa, synthetic construct
  REGION 1..7: note = 3-2 Antibody VL-CDR2
ESNKRPS

SEQ ID NO: 28 - protein, 10 aa, synthetic construct
  REGION 1..10: note = 3-2 Antibody VL-CDR3
GSWDSSNGGI

SEQ ID NO: 29 - protein, 11 aa, synthetic construct
  REGION 1..11: note = 1-65 Antibody VL-CDR1
SGGGSSGYGYG

SEQ ID NO: 30 - protein, 7 aa, synthetic construct
  REGION 1..7: note = 1-65 Antibody VL-CDR2
WNDKRPS

SEQ ID NO: 31 - protein, 14 aa, synthetic construct
  REGION 1..14: note = 1-65 Antibody VL-CDR3
GNDDYSSDSGYVGV

SEQ ID NO: 32 - protein, 5 aa, synthetic construct
  REGION 1..5: note = 1-28 Antibody VL-CDR1
GYGYG

SEQ ID NO: 33 - skipped

SEQ ID NO: 34 - protein, 11 aa, synthetic construct
  REGION 1..11: note = 1-28 Antibody VL-CDR3
GSEDSSTLAGI

SEQ ID NO: 35 - protein, 129 aa, synthetic construct
  REGION 1..129: note = 2-13 Antibody VH
AVTLDESGGGLQTPGGALSLVCKASGFTFSSHGMFWVRQTPGKGLEYVAEITNDGSGTNYGSAVKGRATISRDNGQSTVRLQLNNLRAEDTGTYFCARSTYECPGGFSCWGDTGQIDAWGHGTEVIVSS

SEQ ID NO: 36 - protein, 131 aa, synthetic construct
  REGION 1..131: note = 3-2 Antibody VH
AVTLDESGGGLQTPGGALSLVCKASGFTFSSFNMFWVRQAPGKGLEYVAQISSSGSSTNYAPAVRGRATISRDNGQSTVRLQLNNPGAEDTGTYYCAKSSYDCPYGHCSSGVDSAGEIDAWGHGTEVIVSS

SEQ ID NO: 37 - protein, 122 aa, synthetic construct
  REGION 1..122: note = 1-65 Antibody VH
AVTLDESGGGLQTPGGALSLVCKASGFTFSSYQMGWVRQAPGKGLEWVGVINKSGSDTSYGSAVKGRATISRDNGQSTVRLQLNNLRAEDTGTYFCAKGSASYITAATIDAWGHGTEVIVSS

SEQ ID NO: 38 - protein, 136 aa, synthetic construct
  REGION 1..136: note = 1-28 Antibody VH
AVTLDESGGGLQTPGGALSLVCKASGFDFSDYGMGWVRQAPGKGLEWVAAIRSDGSNPSYGSAVKGRATISKDNGRSTVRLQLNNLRAEDTATYYCAKDGNGYCALDAYRSGGYSCGVYPGSIDAWGHGTEVIVSS

SEQ ID NO: 39 - protein, 104 aa, synthetic construct
  REGION 1..104: note = 2-13 Antibody VL
ALTQPSSVSANPGETVKITCSGGSYSYGWFQQKSPGSALVTVIYWDDERPSDIPSRFSGALSGSTNTLTITGVQADDEAVYFCGTEDISGTAGVFGAGTTLTVL

SEQ ID NO: 40 - protein, 108 aa, synthetic construct
  REGION 1..108: note = 3-2 Antibody VL
ALTQPSSVSANPGETVKITCSGGGSYAGSYYYGWYQQKAPGSAPVTLIYESNKRPSDIPSRFSGSTSGSTATLTITGVQADDEAIYYCGSWDSSNGGIFGAGTTLTVL

SEQ ID NO: 41 - protein, 110 aa, synthetic construct
  REGION 1..110: note = 1-65 Antibody VL
ALTQPSSVSANPGETVKITCSGGGSSGYGYGWYQQKSPSSAPLTVIYWNDKRPSDIPSRFSGSKSGSTHTLTITGVQAEDEAVYFCGNDDYSSDSGYVGVFGAGTTLTVL

SEQ ID NO: 42 - protein, 104 aa, synthetic construct
  REGION 1..104: note = 1-28 Antibody VL
ALTQPSSVSANLEGTVEITCSGSGYGYGWYQQKSPGSAPVTVIYQNDKRPSDIPSRFSGSKSGSTGTLTITGVQVEDEAVYYCGSEDSSTLAGIFGAGTTLTVL

SEQ ID NO: 43 - DNA, 388 bp, synthetic construct
  misc_feature 1..388: note = 2-13 Antibody VH
  source 1..388: mol_type = other DNA
gccgtgacgttggacgagtccgggggcggcctccagacgcccggaggagcgctcagcctcgtctgcaaggcctccgggttcaccttcagcagccatggcatgttctgggtgcgacagacgcccggcaaggggttggaatatgtcgctgaaattaccaatgatggtagtggcacaaactacgggtcggcggtgaagggccgtgccaccatctcgagggacaacgggcagagcacagtgaggctgcagctgaacaacctcagggctgaggacaccggcacctacttctgcgccagatctacttatgaatgtcctggtggttttagttgttggggtgatactggtcaaatagacgcatggggccacgggaccgaagtcatcgtctcctcca

SEQ ID NO: 44 - DNA, 394 bp, synthetic construct
  misc_feature 1..394: note = 3-2 Antibody VH
  source 1..394: mol_type = other DNA
gccgtgacgttggacgagtccgggggcggcctccagacgcccggaggagcgctcagcctcgtctgcaaggcctccgggttcaccttcagcagcttcaacatgttctgggtgcgacaggcgcccggcaaggggctggaatacgtcgctcaaattagcagcagtggtagtagcacaaactacgcacccgcggtgaggggccgtgccaccatctcgagggacaacgggcagagcacagtgaggctgcagctgaacaaccccggggctgaagacaccggcacctactactgcgccaaaagtagttatgactgtccttacggtcattgtagtagtggtgttgatagtgctggtgagatcgacgcatggggccacgggaccgaagtcatcgtctcctcca

SEQ ID NO: 45 - DNA, 366 bp, synthetic construct
  misc_feature 1..366: note = 1-65 Antibody VH
  source 1..366: mol_type = other DNA
gccgtgacgttggacgagtccgggggcggcctccagacgcccggaggagcgctcagcctcgtctgcaaggcctccgggttcaccttcagcagctatcagatgggctgggtgcgacaggcgcccggcaaggggctggaatgggtcggtgttattaacaagagtggtagtgacacatcatacgggtcggcggtgaagggccgtgccaccatctcgagggacaacgggcagagcacagtgaggctgcagctgaacaacctcagggctgaggacaccggcacctacttctgcgccaaaggttctgctagttatataactgctgctaccatcgacgcatggggccacgggaccgaagtcatcgtctcctcc

SEQ ID NO: 46 - DNA, 408 bp, synthetic construct
  misc_feature 1..408: note = 1-28 Antibody VH
  source 1..408: mol_type = other DNA
gccgtgacgttggacgagtccgggggcggcctccagacgcccggaggagcgctcagcctcgtctgcaaggcctccgggttcgacttcagcgattatggcatgggttgggtgcgacaggctccaggcaaggggctggagtgggttgctgctattagaagtgatggtagtaacccatcatacgggtcggcggtgaagggccgtgccaccatctcgaaggacaacgggcgaagcacagtgaggctgcagctgaacaacctcagggctgaggacaccgccacctactactgcgccaaggatggtaatggttactgtgctctcgatgcttatcgtagtggtggttatagttgtggtgtttatcctggtagcatcgacgcatggggccacgggaccgaagtcatcgtctcctcc

SEQ ID NO: 47 - DNA, 315 bp, synthetic construct
  misc_feature 1..315: note = 2-13 Antibody VL
  source 1..315: mol_type = other DNA
ggccctgactcagccgtcctcggtgtcagcaaacccaggagaaaccgtcaagataacctgctccgggggtagctatagctatggctggttccagcagaagtctcctggcagtgcccttgtcactgtgatctactgggatgatgagagaccctcggacatcccttcacgattctccggtgccctatccggctccacaaacacattaaccatcactggggtccaagccgacgacgaggctgtctatttctgtgggactgaagacatcagcggcactgctggtgtatttggggccgggacaaccctgaccgtcctggg

SEQ ID NO: 48 - DNA, 327 bp, synthetic construct
  misc_feature 1..327: note = 3-2 Antibody VL
  source 1..327: mol_type = other DNA
ggccctgactcagccgtcctcggtgtcagcaaacccaggagaaaccgtcaagatcacctgctccgggggtggcagctatgctggaagttactattatggctggtaccagcagaaggcacctggcagtgcccctgtcactctgatctatgaaagcaacaagagaccctcggacatcccttcacgattctccggttccacatctggctccacagccacactaaccatcactggggtccaagccgatgacgaggctatctattactgtgggagctgggacagtagcaatggtggtatatttggggccgggacaaccctgaccgtcctagg

SEQ ID NO: 49 - DNA, 331 bp, synthetic construct
  misc_feature 1..331: note = 1-65 Antibody VL
  source 1..331: mol_type = other DNA
ggccctgactcagccgtcctcggtgtcagcaaaccctggggaaactgtcaagatcacctgctccgggggtggtagcagtggctatggttatggctggtatcagcagaagtcacctagcagtgcccctctcactgtgatctactggaacgacaagagaccctcggacatcccttcacgattctccggttccaaatccggctccacacacacattaaccatcactggggtccaagccgaggacgaggctgtatatttctgtgggaatgatgactacagcagtgatagtggatatgtcggtgtatttggggccgggacaaccctgaccgtccta

SEQ ID NO: 50 - DNA, 312 bp, synthetic construct
  misc_feature 1..312: note = 1-28 Antibody VL
  source 1..312: mol_type = other DNA
gccctgactcagccgtcctcggtgtcagcaaacctggaaggaaccgtcgagatcacctgctccgggagtggctatggttatggctggtatcagcagaagtctcctggcagtgcccctgtcactgtgatctatcagaacgacaagagaccctcggacatcccttcacgattctccggttccaaatccggctccacgggcacattaaccatcactggggtccaagtcgaggacgaggctgtctattactgtgggagtgaagacagcagcactcttgctggtatatttggggccgggacaaccctgaccgtccta

SEQ ID NO: 51 - protein, 100 aa, synthetic construct
  REGION 1..100: note = M3 mutant
QFLKEGQLAAGTCEIVTLDRDSSQPRIEERSQTARCACRKGQIAGTTRARPACVDARIIKTKQWCDMLPCLEGEGCDLLINRSGWTCTQPGGRIKTTTVS

SEQ ID NO: 52 - protein, 100 aa, synthetic construct
  REGION 1..100: note = M4 mutant
QFLKEGQLAAGTCEIVTLDRDSSQPRRTIARQTVKCSCFPGQIAGTTRARPACVDARIIKTKQWCDMLPCLEGEGCDLLINRSGWTCTQPGGRIKTTTVS

SEQ ID NO: 53 - protein, 100 aa, synthetic construct
  REGION 1..100: note = M5 mutant
QFLKEGQLAAGTCEIVTLDRDSSQPRRTIARQTARCACRKGQIAGTTRNKPSCVDARIIKTKQWCDMLPCLEGEGCDLLINRSGWTCTQPGGRIKTTTVS

SEQ ID NO: 54 - protein, 100 aa, synthetic construct
  REGION 1..100: note = M6 mutant
QFLKEGQLAAGTCEIVTLDRDSSQPRRTIARQTARCACRKGQIAGTTRARPACVDARIILQRWWCQMELCLEGEGCDLLINRSGWTCTQPGGRIKTTTVS

SEQ ID NO: 55 - protein, 100 aa, synthetic construct
  REGION 1..100: note = M7 mutant
QFLKEGQLAAGTCEIVTLDRDSSQPRRTIARQTARCACRKGQIAGTTRARPACVDARIIKTKQWCDMLPCLEGEECKTLPDNSGWTCTQPGGRIKTTTVS

SEQ ID NO: 56 - protein, 101 aa, synthetic construct
  REGION 1..101: note = M8 mutant
QFLKEGQLAAGTCEIVTLDRDSSQPRRTIARQTARCACRKGQIAGTTRARPACVDARIIKTKQWCDMLPCLEGEGCDLLINRSGWTCSCSSGNKIKTTTVS

SEQ ID NO: 57 - protein, 459 aa, synthetic construct
  REGION 1..459: note = 2-13 Antibody HC
AVTLDESGGGLQTPGGALSLVCKASGFTFSSHGMFWVRQTPGKGLEYVAEITNDGSGTNYGSAVKGRATISRDNGQSTVRLQLNNLRAEDTGTYFCARSTYECPGGFSCWGDTGQIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 461 aa, synthetic construct
  REGION 1..461: note = 3-2 Antibody HC
AVTLDESGGGLQTPGGALSLVCKASGFTFSSFNMFWVRQAPGKGLEYVAQISSSGSSTNYAPAVRGRATISRDNGQSTVRLQLNNPGAEDTGTYYCAKSSYDCPYGHCSSGVDSAGEIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 452 aa, synthetic construct
  REGION 1..452: note = 1-65 Antibody HC
AVTLDESGGGLQTPGGALSLVCKASGFTFSSYQMGWVRQAPGKGLEWVGVINKSGSDTSYGSAVKGRATISRDNGQSTVRLQLNNLRAEDTGTYFCAKGSASYITAATIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 466 aa, synthetic construct
  REGION 1..466: note = 1-28 Antibody HC
AVTLDESGGGLQTPGGALSLVCKASGFDFSDYGMGWVRQAPGKGLEWVAAIRSDGSNPSYGSAVKGRATISKDNGRSTVRLQLNNLRAEDTATYYCAKDGNGYCALDAYRSGGYSCGVYPGSIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 211 aa, synthetic construct
  REGION 1..211: note = 2-13 Antibody LC
ALTQPSSVSANPGETVKITCSGGSYSYGWFQQKSPGSALVTVIYWDDERPSDIPSRFSGALSGSTNTLTITGVQADDEAVYFCGTEDISGTAGVFGAGTTLTVLRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 215 aa, synthetic construct
  REGION 1..215: note = 3-2 Antibody LC
ALTQPSSVSANPGETVKITCSGGGSYAGSYYYGWYQQKAPGSAPVTLIYESNKRPSDIPSRFSGSTSGSTATLTITGVQADDEAIYYCGSWDSSNGGIFGAGTTLTVLRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 217 aa, synthetic construct
  REGION 1..217: note = 1-65 Antibody LC
ALTQPSSVSANPGETVKITCSGGGSSGYGYGWYQQKSPSSAPLTVIYWNDKRPSDIPSRFSGSKSGSTHTLTITGVQAEDEAVYFCGNDDYSSDSGYVGVFGAGTTLTVLRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 211 aa, synthetic construct
  REGION 1..211: note = 1-28 Antibody LC
ALTQPSSVSANLEGTVEITCSGSGYGYGWYQQKSPGSAPVTVIYQNDKRPSDIPSRFSGSKSGSTGTLTITGVQVEDEAVYYCGSEDSSTLAGIFGAGTTLTVLRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 5 aa, synthetic construct
  REGION 1..5: note = EP1 Epitope
IVTLD

SEQ ID NO: 66 - protein, 5 aa, synthetic construct
  REGION 1..5: note = EP2 Epitope
DSSQP

SEQ ID NO: 67 - protein, 5 aa, synthetic construct
  REGION 1..5: note = EP3 Epitope
RTIAR

SEQ ID NO: 68 - protein, 7 aa, synthetic construct
  REGION 1..7: note = EP4 Epitope
ARCACRK

SEQ ID NO: 69 - protein, 4 aa, synthetic construct
  REGION 1..4: note = EP5 Epitope
ARPA

SEQ ID NO: 70 - protein, 9 aa, synthetic construct
  REGION 1..9: note = EP6 Epitope
KTKQWCDML

SEQ ID NO: 71 - protein, 8 aa, synthetic construct
  REGION 1..8: note = EP7 Epitope
GCDLLINR

SEQ ID NO: 72 - protein, 8 aa, synthetic construct
  REGION 1..8: note = EP8 Epitope
TCTQPGGR

SEQ ID NO: 73 - protein, 20 aa, synthetic construct
  REGION 1..20: note = F2-01-BSA (#1)
TADRDSSQPRRTIARQTARC

SEQ ID NO: 74 - protein, 20 aa, synthetic construct
  REGION 1..20: note = F2-02-BSA (#2)
TLARDSSQPRRTIARQTARC

SEQ ID NO: 75 - protein, 20 aa, synthetic construct
  REGION 1..20: note = F2-03-BSA (#3)
TLDRASSQPRRTIARQTARC

SEQ ID NO: 76 - protein, 20 aa, synthetic construct
  REGION 1..20: note = F2-04-BSA (#4)
TLDRDASQPRRTIARQTARC

SEQ ID NO: 77 - protein, 20 aa, synthetic construct
  REGION 1..20: note = F2-05-BSA (#5)
TLDRDSAQPRRTIARQTARC

SEQ ID NO: 78 - protein, 20 aa, synthetic construct
  REGION 1..20: note = F2-06-BSA (#6)
TLDRDSSAPRRTIARQTARC

SEQ ID NO: 79 - protein, 20 aa, synthetic construct
  REGION 1..20: note = F2-07-BSA (#7)
TLDRDSSQARRTIARQTARC

SEQ ID NO: 80 - protein, 20 aa, synthetic construct
  REGION 1..20: note = F2-08-BSA (#8)
TLDRDSSQPRATIARQTARC

SEQ ID NO: 81 - protein, 20 aa, synthetic construct
  REGION 1..20: note = F2-09-BSA (#9)
TLDRDSSQPRRAIARQTARC

SEQ ID NO: 82 - protein, 20 aa, synthetic construct
  REGION 1..20: note = F2-10-BSA (#10)
TLDRDSSQPRRTAARQTARC

SEQ ID NO: 83 - protein, 20 aa, synthetic construct
  REGION 1..20: note = F2-11-BSA (#11)
TLDRDSSQPRRTIRRQTARC

SEQ ID NO: 84 - protein, 20 aa, synthetic construct
  REGION 1..20: note = F2-12-BSA (#12)
TLDRDSSQPRRTIAAQTARC

SEQ ID NO: 85 - protein, 20 aa, synthetic construct
  REGION 1..20: note = F2-12-BSA (#13)
TLDRDSSQPRRTIARQTVRC

SEQ ID NO: 86 - protein, 100 aa, synthetic construct
  REGION 1..100: note = Wild-type FAM19A5 Isoform 2 (without signal peptide)
QFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 87 - protein, 100 aa, synthetic construct
  REGION 1..100: note = M1 mutant
QFLKEGQLAAGTCEVIAAHRDSSQPRRTIARQTARCACRKGQIAGTTRARPACVDARIIKTKQWCDMLPCLEGEGCDLLINRSGWTCTQPGGRIKTTTVS

SEQ ID NO: 88 - protein, 100 aa, synthetic construct
  REGION 1..100: note = M2 mutant
QFLKEGQLAAGTCEIVTLDRCCNKNRRTIARQTARCACRKGQIAGTTRARPACVDARIIKTKQWCDMLPCLEGEGCDLLINRSGWTCTQPGGRIKTTTVS

SEQ ID NO: 89 - protein, 5 aa, synthetic construct
  REGION 1..5: note = P2-C12 antibodyVH CDR1
TYAVT

SEQ ID NO: 90 - protein, 16 aa, synthetic construct
  REGION 1..16: note = P2-C12antibodyVH CDR2
YINWRGGTSYANWAKG

SEQ ID NO: 91 - protein, 15 aa, synthetic construct
  REGION 1..15: note = P2-C12antibodyVH CDR3
DASSGAAFGSYGMDP

SEQ ID NO: 92 - protein, 11 aa, synthetic construct
  REGION 1..11: note = P2-C12 antibodyVL CDR1
QASQSISSYLS

SEQ ID NO: 93 - protein, 7 aa, synthetic construct
  REGION 1..7: note = P2-C12antibodyVL CDR2
EASKLAS

SEQ ID NO: 94 - protein, 12 aa, synthetic construct
  REGION 1..12: note = P2-C12antibodyVL CDR3
QQGYSSTNVWNA

SEQ ID NO: 95 - protein, 6 aa, synthetic construct
  REGION 1..6: note = 13B4antibodyVH CDR1
SSNWWS

SEQ ID NO: 96 - protein, 16 aa, synthetic construct
  REGION 1..16: note = 13B4antibodyVH CDR2
EIYHGGTTNYNPSLKG

SEQ ID NO: 97 - protein, 9 aa, synthetic construct
  REGION 1..9: note = 13B4antibodyVH CDR3
WQLVGGLDV

SEQ ID NO: 98 - protein, 11 aa, synthetic construct
  REGION 1..11: note = 13B4antibodyVL CDR1
SGDKLGNVYAS

SEQ ID NO: 99 - protein, 7 aa, synthetic construct
  REGION 1..7: note = 13B4antibodyVL CDR2
QDNKRPS

SEQ ID NO: 100 - protein, 9 aa, synthetic construct
  REGION 1..9: note = 13B4antibodyVL CDR3
QAWDSSTAV

SEQ ID NO: 101 - protein, 5 aa, synthetic construct
  REGION 1..5: note = 13F7antibodyVH CDR1
GYSWT

SEQ ID NO: 102 - protein, 16 aa, synthetic construct
  REGION 1..16: note = 13F7antibodyVH CDR2
EISHFGSANYNPSLKS

SEQ ID NO: 103 - protein, 15 aa, synthetic construct
  REGION 1..15: note = 13F7antibodyVH CDR3
ALRGTYSRFYYGMDV

SEQ ID NO: 104 - protein, 16 aa, synthetic construct
  REGION 1..16: note = 13F7antibodyVL CDR1
RSSQSLLHSNGYNYLD

SEQ ID NO: 105 - protein, 7 aa, synthetic construct
  REGION 1..7: note = 13F7antibodyVL CDR2
LGSNRAS

SEQ ID NO: 106 - protein, 9 aa, synthetic construct
  REGION 1..9: note = 13F7antibodyVL CDR3
MQARQTPLT

SEQ ID NO: 107 - protein, 5 aa, synthetic construct
  REGION 1..5: note = 15A9antibodyVH CDR1
SYYWS

SEQ ID NO: 108 - protein, 16 aa, synthetic construct
  REGION 1..16: note = 15A9antibodyVH CDR2
YIYPSGSTNYNPSLKS

SEQ ID NO: 109 - protein, 12 aa, synthetic construct
  REGION 1..12: note = 15A9antibodyVH CDR3
VNPFGYYYAMDV

SEQ ID NO: 110 - protein, 11 aa, synthetic construct
  REGION 1..11: note = 15A9antibodyVL CDR1
RASQSISTSLN

SEQ ID NO: 111 - protein, 7 aa, synthetic construct
  REGION 1..7: note = 15A9antibodyVL CDR2
GASTLQS

SEQ ID NO: 112 - protein, 9 aa, synthetic construct
  REGION 1..9: note = 15A9antibodyVL CDR3
QESASIPRT

SEQ ID NO: 113 - protein, 5 aa, synthetic construct
  REGION 1..5: note = P1-A03antibodyVH CDR1
SDYMS

SEQ ID NO: 114 - protein, 16 aa, synthetic construct
  REGION 1..16: note = P1-A03antibodyVH CDR2
IIYPSTTTYYASWAKG

SEQ ID NO: 115 - protein, 10 aa, synthetic construct
  REGION 1..10: note = P1-A03antibodyVH CDR3
GSNWSSGMNL

SEQ ID NO: 116 - protein, 11 aa, synthetic construct
  REGION 1..11: note = P1-A03antibodyVL CDR1
LASEDIYSGIS

SEQ ID NO: 117 - protein, 7 aa, synthetic construct
  REGION 1..7: note = P1-A03antibodyVL CDR2
GASNLES

SEQ ID NO: 118 - protein, 12 aa, synthetic construct
  REGION 1..12: note = P1-A03antibodyVL CDR3
LGGYSYSSTGLT

SEQ ID NO: 119 - protein, 5 aa, synthetic construct
  REGION 1..5: note = P1-A08antibodyVH CDR1
TYYMS

SEQ ID NO: 120 - protein, 16 aa, synthetic construct
  REGION 1..16: note = P1-A08antibodyVH CDR2
IVYPSGTTYYANWAKG

SEQ ID NO: 121 - protein, 8 aa, synthetic construct
  REGION 1..8: note = P1-A08antibodyVH CDR3
GDSFGYGL

SEQ ID NO: 122 - protein, 11 aa, synthetic construct
  REGION 1..11: note = P1-A08antibodyVL CDR1
TADTLSRSYAS

SEQ ID NO: 123 - protein, 7 aa, synthetic construct
  REGION 1..7: note = P1-A08antibodyVL CDR2
RDTSRPS

SEQ ID NO: 124 - protein, 13 aa, synthetic construct
  REGION 1..13: note = P1-A08antibodyVL CDR3
ATSDGSGSNYQYV

SEQ ID NO: 125 - protein, 5 aa, synthetic construct
  REGION 1..5: note = P1-F02antibodyVH CDR1
NYYMG

SEQ ID NO: 126 - protein, 16 aa, synthetic construct
  REGION 1..16: note = P1-F02antibodyVH CDR2
IIYASGSTYYASWAKG

SEQ ID NO: 127 - protein, 17 aa, synthetic construct
  REGION 1..17: note = P1-F02antibodyVH CDR3
IDIGVGDYGWAYDRLDL

SEQ ID NO: 128 - protein, 11 aa, synthetic construct
  REGION 1..11: note = P1-F02antibodyVL CDR1
LASEDIYSGIS

SEQ ID NO: 129 - protein, 7 aa, synthetic construct
  REGION 1..7: note = P1-F02antibodyVL CDR2
GASNLES

SEQ ID NO: 130 - protein, 10 aa, synthetic construct
  REGION 1..10: note = P1-F02antibodyVL CDR3
LGGYSYSSIT

SEQ ID NO: 131 - protein, 5 aa, synthetic construct
  REGION 1..5: note = P2-A01antibodyVH CDR1
GYYMS

SEQ ID NO: 132 - protein, 16 aa, synthetic construct
  REGION 1..16: note = P2-A01antibodyVH CDR2
IIYPSGSTDYASWAKG

SEQ ID NO: 133 - protein, 15 aa, synthetic construct
  REGION 1..15: note = P2-A01antibodyVH CDR3
VAGYVGYGYETFFDI

SEQ ID NO: 134 - protein, 11 aa, synthetic construct
  REGION 1..11: note = P2-A01antibodyVL CDR1
LASEDIYSGIS

SEQ ID NO: 135 - protein, 7 aa, synthetic construct
  REGION 1..7: note = P2-A01antibodyVL CDR2
GASNLES

SEQ ID NO: 136 - protein, 14 aa, synthetic construct
  REGION 1..14: note = P2-A01antibodyVL CDR3
LGGVTYSSTGTHLT

SEQ ID NO: 137 - protein, 5 aa, synthetic construct
  REGION 1..5: note = P2-A03antibodyVH CDR1
NYDMS

SEQ ID NO: 138 - protein, 16 aa, synthetic construct
  REGION 1..16: note = P2-A03antibodyVH CDR2
FMDTDGSAYYATWAKG

SEQ ID NO: 139 - protein, 11 aa, synthetic construct
  REGION 1..11: note = P2-A03antibodyVH CDR3
RGSSYYGGIDI

SEQ ID NO: 140 - protein, 11 aa, synthetic construct
  REGION 1..11: note = P2-A03antibodyVL CDR1
QASQSIGGNLA

SEQ ID NO: 141 - protein, 7 aa, synthetic construct
  REGION 1..7: note = P2-A03antibodyVL CDR2
RASTLAS

SEQ ID NO: 142 - protein, 14 aa, synthetic construct
  REGION 1..14: note = P2-A03antibodyVL CDR3
QSPAYDPAAYVGNA

SEQ ID NO: 143 - protein, 5 aa, synthetic construct
  REGION 1..5: note = P2-F07antibodyVH CDR1
SYYMN

SEQ ID NO: 144 - protein, 16 aa, synthetic construct
  REGION 1..16: note = P2-F07antibodyVH CDR2
IIYPSGTTYYAGWAKG

SEQ ID NO: 145 - protein, 8 aa, synthetic construct
  REGION 1..8: note = P2-F07antibodyVH CDR3
TVSGYFDI

SEQ ID NO: 146 - protein, 11 aa, synthetic construct
  REGION 1..11: note = P2-F07antibodyVL CDR1
LASEDIYSALA

SEQ ID NO: 147 - protein, 7 aa, synthetic construct
  REGION 1..7: note = P2-F07antibodyVL CDR2
GTSNLES

SEQ ID NO: 148 - protein, 9 aa, synthetic construct
  REGION 1..9: note = P2-F07antibodyVL CDR3
QGYSSYPLT

SEQ ID NO: 149 - protein, 5 aa, synthetic construct
  REGION 1..5: note = P2-F11antibodyVH CDR1
SYGVS

SEQ ID NO: 150 - protein, 16 aa, synthetic construct
  REGION 1..16: note = P2-F11antibodyVH CDR2
YIANNYNPHYASWAKG

SEQ ID NO: 151 - protein, 7 aa, synthetic construct
  REGION 1..7: note = P2-F11antibodyVH CDR3
DNYGMDP

SEQ ID NO: 152 - protein, 13 aa, synthetic construct
  REGION 1..13: note = P2-F11antibodyVL CDR1
QASQSVYNNKNLA

SEQ ID NO: 153 - protein, 7 aa, synthetic construct
  REGION 1..7: note = P2-F11antibodyVL CDR2
AASTLAS

SEQ ID NO: 154 - protein, 13 aa, synthetic construct
  REGION 1..13: note = P2-F11antibodyVL CDR3
QGEFSCSSADCNA

SEQ ID NO: 155 - protein, 121 aa, synthetic construct
  REGION 1..121: note = P2-C12antibodyVH
QSLEESGGRLVTPGTPLTLTCTVSGFSLSTYAVTWVRQAPGKGLEWIGYINWRGGTSYANWAKGRFTISKTSSTTVDLKMTSPTTEDTATYFCARDASSGAAFGSYGMDPWGPGTLVTVSS

SEQ ID NO: 156 - protein, 118 aa, synthetic construct
  REGION 1..118: note = 13B4antibodyVH
QVQLQESGPGLVKPSGTLSLNCAVSGGSISSSNWWSWVRQPPGKGLEWIGEIYHGGTTNYNPSLKGRVTMSVDKTKNQFSLRLSSVTAVDTAVYYCARWQLVGGLDVWGQGTTVTVSS

SEQ ID NO: 157 - protein, 123 aa, synthetic construct
  REGION 1..123: note = 13F7antibodyVH
QVQLQEWGAGLLKPSETLSLTCAINAESFNGYSWTWIRQTPGKGLEWIGEISHFGSANYNPSLKSRATISADKSKNQFSLKLTSVTAVDTAVYYCARALRGTYSRFYYGMDVWGQGTTVTVSS

SEQ ID NO: 158 - protein, 120 aa, synthetic construct
  REGION 1..120: note = 15A9antibodyVH
QVQLQESGPGLVKPSETLSLTCTVSGGSISSYYWSWIRQPPGKGLEWIGYIYPSGSTNYNPSLKSRVTISVDTSKNQFSLNLKSVTAVDTAVYYCARVNPFGYYYAMDVWGQGTTVTVSS

SEQ ID NO: 159 - protein, 116 aa, synthetic construct
  REGION 1..116: note = P1-A03antibodyVH
QSVEESGGRLVTPGTPLTLTCTVSGFSLSSDYMSWVRQAPGEGLEWIGIIYPSTTTYYASWAKGRFTISKTSSTTVELKMTSLTTEDTATYFCARGSNWSSGMNLWGPGTLVTVSS

SEQ ID NO: 160 - protein, 113 aa, synthetic construct
  REGION 1..113: note = P1-A08antibodyVH
QSLEESGGRLVTPGTPLTLTCTASGFSLSTYYMSWVRQAPGKGLEWIGIVYPSGTTYYANWAKGRFTISTASTTVDLMITSPTTEDTATYFCARGDSFGYGLWGPGTLVTVSS

SEQ ID NO: 161 - protein, 122 aa, synthetic construct
  REGION 1..122: note = P1-F02antibodyVH
QSLEESGGRLVTPGTPLTLTCTASGFSLSNYYMGWVRQAPGEGLEWIGIIYASGSTYYASWAKGRFTISKTSTTVDLKMTSLTTEDTATYFCARIDIGVGDYGWAYDRLDLWGQGTLVTVSS

SEQ ID NO: 162 - protein, 121 aa, synthetic construct
  REGION 1..121: note = P2-A01antibodyVH
QEQLVESGGRLVTPGTPLTLSCTASGFFLSGYYMSWVRQAPGKGLEWIGIIYPSGSTDYASWAKGRFTISKTSTTVDLKITTPTTEDTATYFCARVAGYVGYGYETFFDIWGPGTLVTVSL

SEQ ID NO: 163 - protein, 116 aa, synthetic construct
  REGION 1..116: note = P2-A03antibodyVH
QSVEESGGRLVTPGTPLTLTCTVSGFSLNNYDMSWVRQAPGKGLEYIGFMDTDGSAYYATWAKGRFTISRTSTTVDLKMTSPTTEDTATYFCARRGSSYYGGIDIWGPGTPVTVSL

SEQ ID NO: 164 - protein, 113 aa, synthetic construct
  REGION 1..113: note = P2-F07antibodyVH
QSLEESGGRLVTPGTPLTLTCTASGFSLSSYYMNWVRQAPGKGLEWIGIIYPSGTTYYAGWAKGRFTISKTSTTVDLKITSPTSEDTATYFCARTVSGYFDIWGPGTLVTVSL

SEQ ID NO: 165 - protein, 114 aa, synthetic construct
  REGION 1..114: note = P2-F11antibodyVH
QEQLVESGGRLVTPGTTLTLTCTVSGFSLSSYGVSWVRQAPGKGLEWIGYIANNYNPHYASWAKGRFTISKTSSTTVDLKMTSLTTEDTATYFCARDNYGMDPWGPGTLVTVSS

SEQ ID NO: 166 - protein, 110 aa, synthetic construct
  REGION 1..110: note = P2-C12antibodyVL
ELDMTQTPSSVSAAVGGTVTIKCQASQSISSYLSWYQQKPGQPPKLLIYEASKLASGVPSRFSGSGYGTEFTLTISDLECADAATYYCQQGYSSTNVWNAFGGGTNVEIK

SEQ ID NO: 167 - protein, 106 aa, synthetic construct
  REGION 1..106: note = 13B4antibodyVL
SYELTQPLSVSVSPGQTASITCSGDKLGNVYASWYQQKPGQSPTLVIYQDNKRPSGIPERFSGSNSGKTATLTISGTQALDEADYYCQAWDSSTAVFGGGTKLTVL

SEQ ID NO: 168 - protein, 112 aa, synthetic construct
  REGION 1..112: note = 13F7antibodyVL
DIVMTQTPLSLPVAPGEPASISCRSSQSLLHSNGYNYLDWYVQKPGQPPQLLIYLGSNRASGVPDRFSGSGSGTDFTLKISRVEAEDVGVYYCMQARQTPLTFGGGTKVEIK

SEQ ID NO: 169 - protein, 107 aa, synthetic construct
  REGION 1..107: note = 15A9antibodyVL
DIQMTQSPSSLSASVGDRITISCRASQSISTSLNWYQQTPGKAPRLLIYGASTLQSGVPSRFSGGGSGTDFSLTITSLQPEDFATYYCQESASIPRTFGQGTKLDIK

SEQ ID NO: 170 - protein, 110 aa, synthetic construct
  REGION 1..110: note = P1-A03antibodyVL
ELVMTQTPPSLSASVGETVRIRCLASEDIYSGISWYQQKPEKPPTLLISGASNLESGVPPRFSGSGSGTDYTLTIGGVQAEDAATYYCLGGYSYSSTGLTFGAGTNVEIK

SEQ ID NO: 171 - protein, 110 aa, synthetic construct
  REGION 1..110: note = P1-A08antibodyVL
ELVLTQSPSVQVNLGQTVSLTCTADTLSRSYASWYQQKPGQAPVLLIYRDTSRPSGVPDRFSGSSSGNTATLTISGAQAGDEADYYCATSDGSGSNYQYVFGGGTQLTVT

SEQ ID NO: 172 - protein, 108 aa, synthetic construct
  REGION 1..108: note = P1-F02antibodyVL
ELDMTQTPPSLSASVGETVRIRCLASEDIYSGISWYQQKPGKPPTLLIYGASNLESGVPPRFSGSGSGTDYTLTIGGVQAEDAATYYCLGGYSYSSITFGAGTNVEIK

SEQ ID NO: 173 - protein, 112 aa, synthetic construct
  REGION 1..112: note = P2-A01antibodyVL
ELVMTQTPPSLSASVGETVRIRCLASEDIYSGISWYQQKPGKPPTLLIYGASNLESGVPPRFSGSGSGSDYTLTIGGVQAEDAATYYCLGGVTYSSTGTHLTFGAGTNVEIK

SEQ ID NO: 174 - protein, 112 aa, synthetic construct
  REGION 1..112: note = P2-A03antibodyVL
ELDLTQTPASVSEPVGGTVTIKCQASQSIGGNLAWYQQKPGQPPKLLIYRASTLASGVPSRFKGSGSGTDFTLTISDLECADAATYYCQSPAYDPAAYVGNAFGGGTELEIL

SEQ ID NO: 175 - protein, 107 aa, synthetic construct
  REGION 1..107: note = P2-F07antibodyVL
ELDLTQTPPSLSASVGGTVTINCLASEDIYSALAWYQQKPGKPPTLLISGTSNLESGVPPRFSGSGSGTDYTLTIGGVQAEDAATYFCQGYSSYPLTFGAGTNVEIK

SEQ ID NO: 176 - protein, 113 aa, synthetic construct
  REGION 1..113: note = P2-F11antibodyVL
ELDLTQTPSSVSAAVGGTVTINCQASQSVYNNKNLAWYQQKPGQPPKLLIYAASTLASGVSSRFKGSGSGTQFTLTISDVQCDDAATYYCQGEFSCSSADCNAFGGGTELEIL

SEQ ID NO: 177 - DNA, 363 bp, synthetic construct
  misc_feature 1..363: note = P2-C12antibodyVH
  source 1..363: mol_type = other DNA
cagtcgctggaggagtccgggggtcgcctggtcacgcctgggacacccctgacactcacctgcaccgtctctggattctccctcagtacctatgcagtgacctgggtccgccaggctccagggaaggggctggaatggatcggatacattaattggcgtggtgggacatcctacgcgaactgggcgaaaggccgattcaccatctccaaaacctcgtcgaccacggtggatctgaaaatgaccagtccgacaaccgaggacacggccacctatttctgtgccagagatgctagtagtggtgctgcttttgggtcttacggcatggacccctggggcccagggaccctcgtcaccgtctcttca

SEQ ID NO: 178 - DNA, 330 bp, synthetic construct
  misc_feature 1..330: note = P2-C12antibodyVL
  source 1..330: mol_type = other DNA
gagctcgatatgacccagactccatcctccgtgtctgcagctgtgggaggcacagtcaccatcaagtgccaggccagtcagagcattagtagctacttatcctggtatcagcagaaaccagggcagcctcccaagctcctgatctatgaagcatccaaactggcctctggggtcccatcgcggttcagcggcagtggatatgggacagagttcactctcaccatcagcgacctggagtgtgccgatgctgccacttactactgtcaacagggttatagtagtactaatgtttggaatgctttcggcggaggcaccaatgtggaaatcaaa

SEQ ID NO: 179 - protein, 451 aa, synthetic construct
  REGION 1..451: note = P2-C12, HC
QSLEESGGRLVTPGTPLTLTCTVSGFSLSTYAVTWVRQAPGKGLEWIGYINWRGGTSYANWAKGRFTISKTSSTTVDLKMTSPTTEDTATYFCARDASSGAAFGSYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 448 aa, synthetic construct
  REGION 1..448: note = 13B4, HC
QVQLQESGPGLVKPSGTLSLNCAVSGGSISSSNWWSWVRQPPGKGLEWIGEIYHGGTTNYNPSLKGRVTMSVDKTKNQFSLRLSSVTAVDTAVYYCARWQLVGGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 453 aa, synthetic construct
  REGION 1..453: note = 13F7, HC
QVQLQEWGAGLLKPSETLSLTCAINAESFNGYSWTWIRQTPGKGLEWIGEISHFGSANYNPSLKSRATISADKSKNQFSLKLTSVTAVDTAVYYCARALRGTYSRFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 450 aa, synthetic construct
  REGION 1..450: note = 15A9, HC
QVQLQESGPGLVKPSETLSLTCTVSGGSISSYYWSWIRQPPGKGLEWIGYIYPSGSTNYNPSLKSRVTISVDTSKNQFSLNLKSVTAVDTAVYYCARVNPFGYYYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 446 aa, synthetic construct
  REGION 1..446: note = P1-A03, HC
QSVEESGGRLVTPGTPLTLTCTVSGFSLSSDYMSWVRQAPGEGLEWIGIIYPSTTTYYASWAKGRFTISKTSSTTVELKMTSLTTEDTATYFCARGSNWSSGMNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 443 aa, synthetic construct
  REGION 1..443: note = P1-A08, HC
QSLEESGGRLVTPGTPLTLTCTASGFSLSTYYMSWVRQAPGKGLEWIGIVYPSGTTYYANWAKGRFTISTASTTVDLMITSPTTEDTATYFCARGDSFGYGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 452 aa, synthetic construct
  REGION 1..452: note = P1-F02, HC
QSLEESGGRLVTPGTPLTLTCTASGFSLSNYYMGWVRQAPGEGLEWIGIIYASGSTYYASWAKGRFTISKTSTTVDLKMTSLTTEDTATYFCARIDIGVGDYGWAYDRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 451 aa, synthetic construct
  REGION 1..451: note = P2-A01, HC
QEQLVESGGRLVTPGTPLTLSCTASGFFLSGYYMSWVRQAPGKGLEWIGIIYPSGSTDYASWAKGRFTISKTSTTVDLKITTPTTEDTATYFCARVAGYVGYGYETFFDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 446 aa, synthetic construct
  REGION 1..446: note = P2-A03, HC
QSVEESGGRLVTPGTPLTLTCTVSGFSLNNYDMSWVRQAPGKGLEYIGFMDTDGSAYYATWAKGRFTISRTSTTVDLKMTSPTTEDTATYFCARRGSSYYGGIDIWGPGTPVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 443 aa, synthetic construct
  REGION 1..443: note = P2-F07, HC
QSLEESGGRLVTPGTPLTLTCTASGFSLSSYYMNWVRQAPGKGLEWIGIIYPSGTTYYAGWAKGRFTISKTSTTVDLKITSPTSEDTATYFCARTVSGYFDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 444 aa, synthetic construct
  REGION 1..444: note = P2-F11, HC
QEQLVESGGRLVTPGTTLTLTCTVSGFSLSSYGVSWVRQAPGKGLEWIGYIANNYNPHYASWAKGRFTISKTSSTTVDLKMTSLTTEDTATYFCARDNYGMDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 217 aa, synthetic construct
  REGION 1..217: note = P2-C12, LC
ELDMTQTPSSVSAAVGGTVTIKCQASQSISSYLSWYQQKPGQPPKLLIYEASKLASGVPSRFSGSGYGTEFTLTISDLECADAATYYCQQGYSSTNVWNAFGGGTNVEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 213 aa, synthetic construct
  REGION 1..213: note = 13B4, LC
SYELTQPLSVSVSPGQTASITCSGDKLGNVYASWYQQKPGQSPTLVIYQDNKRPSGIPERFSGSNSGKTATLTISGTQALDEADYYCQAWDSSTAVFGGGTKLTVLRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 219 aa, synthetic construct
  REGION 1..219: note = 13F7, LC
DIVMTQTPLSLPVAPGEPASISCRSSQSLLHSNGYNYLDWYVQKPGQPPQLLIYLGSNRASGVPDRFSGSGSGTDFTLKISRVEAEDVGVYYCMQARQTPLTFGGGTKVEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 214 aa, synthetic construct
  REGION 1..214: note = 15A9, LC
DIQMTQSPSSLSASVGDRITISCRASQSISTSLNWYQQTPGKAPRLLIYGASTLQSGVPSRFSGGGSGTDFSLTITSLQPEDFATYYCQESASIPRTFGQGTKLDIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 217 aa, synthetic construct
  REGION 1..217: note = P1-A03, LC
ELVMTQTPPSLSASVGETVRIRCLASEDIYSGISWYQQKPEKPPTLLISGASNLESGVPPRFSGSGSGTDYTLTIGGVQAEDAATYYCLGGYSYSSTGLTFGAGTNVEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 217 aa, synthetic construct
  REGION 1..217: note = P1-A08, LC
ELVLTQSPSVQVNLGQTVSLTCTADTLSRSYASWYQQKPGQAPVLLIYRDTSRPSGVPDRFSGSSSGNTATLTISGAQAGDEADYYCATSDGSGSNYQYVFGGGTQLTVTRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 215 aa, synthetic construct
  REGION 1..215: note = P1-F02, LC
ELDMTQTPPSLSASVGETVRIRCLASEDIYSGISWYQQKPGKPPTLLIYGASNLESGVPPRFSGSGSGTDYTLTIGGVQAEDAATYYCLGGYSYSSITFGAGTNVEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 219 aa, synthetic construct
  REGION 1..219: note = P2-A01, LC
ELVMTQTPPSLSASVGETVRIRCLASEDIYSGISWYQQKPGKPPTLLIYGASNLESGVPPRFSGSGSGSDYTLTIGGVQAEDAATYYCLGGVTYSSTGTHLTFGAGTNVEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 219 aa, synthetic construct
  REGION 1..219: note = P2-A03, LC
ELDLTQTPASVSEPVGGTVTIKCQASQSIGGNLAWYQQKPGQPPKLLIYRASTLASGVPSRFKGSGSGTDFTLTISDLECADAATYYCQSPAYDPAAYVGNAFGGGTELEILRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 214 aa, synthetic construct
  REGION 1..214: note = P2-F07, LC
ELDLTQTPPSLSASVGGTVTINCLASEDIYSALAWYQQKPGKPPTLLISGTSNLESGVPPRFSGSGSGTDYTLTIGGVQAEDAATYFCQGYSSYPLTFGAGTNVEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 220 aa, synthetic construct
  REGION 1..220: note = P2-F11, LC
ELDLTQTPSSVSAAVGGTVTINCQASQSVYNNKNLAWYQQKPGQPPKLLIYAASTLASGVSSRFKGSGSGTQFTLTISDVQCDDAATYYCQGEFSCSSADCNAFGGGTELEILRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC